(19) United States
(12) Patent Application Publication
(10) Pub. No.: US 2024/0368304 A1
(43) Pub. Date: Nov. 7, 2024

(54) HUMANIZED F77 ANTIBODIES AND FRAGMENTS THEREOF

(71) Applicant: The Trustees of the University of Pennsylvania, Philadelphia, PA (US)

(72) Inventors: Mark I. Greene, Penn Valley, PA (US); Hongtao Zhang, Paoli, PA (US); Zhiqiang Zhu, Philadelphia, PA (US); Zheng Cai, Wynnewood, PA (US); Payal Grover, Philadelphia, PA (US); Michael Milone, Moorestown, NJ (US); Selene Nunez-Cruz, Philadelphia, PA (US); Atrish Bagchi, San Francisco, CA (US)

(21) Appl. No.: 18/654,958

(22) Filed: May 3, 2024

Related U.S. Application Data
(60) Provisional application No. 63/500,038, filed on May 4, 2023.
(60) Provisional application No. 63/582,180, filed on Sep. 12, 2023.

(51) Int. Cl. C07K 16/30; A61K 39/00; A61P 35/00; C07K 14/705; C07K 14/725

(52) U.S. Cl. CPC C07K 16/3069; A61K 39/4611; A61K 39/4631; A61K 39/464495; A61P 35/00; C07K 14/7051; C07K 14/70517; C07K 14/70521; C07K 14/70578; A61K 2239/13; A61K 2239/58; C07K 2317/24; C07K 2317/55; C07K 2317/622; C07K 2319/03

(57) ABSTRACT

Disclosed herein are clones of murine F77 monoclonal antibodies, humanized F77 antibodies, single chain variable fragments (scFvs) and single chain antigen binding fragments (scFabs) of the antibodies, and chimeric antigen receptors. The humanized F77 antibodies and their fragments retain the binding specificity of the murine F77 monoclonal antibody to PC-3 cells. The humanized F77 antibodies and their fragments can be used for diagnostic and/or therapeutic tools in diagnosis or treatment of localized (stages I and II), locally advanced (stage III), or advanced (stage IV) prostate cancer.

### CROSS-REFERENCE TO RELATED APPLICATIONS

This application claims priority to and the benefit of U.S. Provisional Patent Application No. 63/500,038, filed May 4, 2023, and U.S. Provisional Patent Application No. 63/582,180, filed Sep. 12, 2023, the disclosures of each of which are incorporated herein by reference in their entirety.

### GOVERNMENT RIGHTS

This invention was made with government support under CA168925 awarded by the National Institutes of Health. The government has certain rights in the invention.

### SEQUENCE LISTING

The instant application contains a Sequence Listing which is being submitted herewith electronically in XML format and is hereby incorporated by reference in its entirety. Said XML copy, created on May 3, 2024, is named 103241007055_Sequence_Listing.xml and is 61,383 bytes in size.

### TECHNICAL FIELD

Disclosed herein are immunotherapeutics, including prostate-cancer specific antibodies, prostate-cancer specific humanized antibodies, and fragments thereof.

### BACKGROUND

The antibodies currently available for detection and treatment of prostate cancers are limited. The monoclonal antibody (mAb) 7E11-C5.3, which binds to prostate-specific membrane antigen (PSMA), has been developed for clinical trials (Bander N H, et al., J Clin Oncol 23:4591-4601 (2005)). The ProstaScint scan (Cytogen), based on ¹¹¹In-labeled 7E11-C5.3, appears superior to the conventional imaging methods for soft-tissue disease, but has limitations because it binds to the intracellular domain on PSMA (Morris M J, et al. Clin Cancer Res 11:7454-7461 (2005)). In addition, PSMA is not expressed in certain advanced, androgen-independent tumor cells, such as PC3 and Du145, and therefore this antibody is not useful for imaging bone metastases. Recent studies show that the anti-prostate stem cell antigen (PSCA) mAb1G8 can inhibit tumor growth of androgen-dependent tumor xenografts (Reiter R E, et al., Proc Natl Acad Sci USA 95:1735-1740 (1998)). However, anti-PSCA mAbs are usually ineffective against androgen independent tumors, which generally do not express PSCA (Saffran D C, et al., Proc Natl Acad Sci USA 98:2658-2663 (2001)). An analysis of prostate cancer tissue sections demonstrated that PSCA is absent in approximately 20% of specimens (Wente M N, et al., Pancreas 31:119-125 (2005)).

A large percentage of androgen-independent prostatic carcinomas metastasize to bone. These metastases are difficult to treat and contribute to increased morbidity and mortality. The PC3 cell line was originally derived from advanced androgen-independent bone metastasis and has become a commonly used cell model for studying androgen-independent prostate cancer.

However, there are no antibody-based treatments for androgen-dependent and androgen-independent prostate cancers.

### SUMMARY

Provided herein are clones of murine F77 monoclonal antibody, humanized F77 antibodies, as well as antigen-binding fragments thereof. In some embodiments, the antigen-binding fragments are single chain variable fragments (scFvs) and single chain antigen binding fragments (scFabs) of clones of the murine F77 monoclonal antibody or the humanized F77 antibodies. The antibodies and the antigen-binding fragments thereof can include a heavy chain variable region and a light chain variable region, wherein:
the heavy chain variable region comprises heavy chain complementarity determining region 1 (CDR1), CDR2, and CDR3 of SEQ ID NOs: 8, 9, and 11, and the light chain variable region comprises light chain CDR1, CDR2, and CDR3 of SEQ ID NOs: 12, 14, and 15;
the heavy chain variable region comprises heavy chain CDR1, CDR2, and CDR3 of SEQ ID NOs: 7, 9, and 11, and the light chain variable region comprises light chain CDR1, CDR2, and CDR3 of SEQ ID NOs: 12, 14, and 15; or
the heavy chain variable region comprises heavy chain CDR1, CDR2, and CDR3 of SEQ ID NOs: 7, 10, and 11, and the light chain variable region comprises light chain CDR1, CDR2, and CDR3 of SEQ ID NOs: 13, 14, and 16.

The antibodies or the antigen-binding fragments thereof may include a heavy chain variable region comprising SEQ ID NO: 1 and a light chain variable region comprising SEQ ID NO: 2.

The antibodies or the antigen-binding fragments thereof may include a heavy chain variable region comprising SEQ ID NO: 3 and a light chain variable region comprising SEQ ID NO: 4.

The antibodies or the antigen-binding fragments thereof may include a heavy chain variable region comprising SEQ ID NO: 5 and a light chain variable region comprising SEQ ID NO: 6.

In some embodiments, the antibodies and the antigen-binding fragments thereof are humanized. For example, the heavy chain variable region, the light chain variable region, or both the heavy chain variable region and the light chain variable region of the antibody or the antigen-binding fragment may be humanized. In some embodiments, the humanized antibody or the humanized antigen-binding fragment comprises a human constant region.

In some embodiments, the humanized antibody or the humanized antigen-binding fragment comprises a heavy chain variable region comprising SEQ ID NO: 17 and a light chain variable region comprising SEQ ID NO: 18. In some embodiments, the heavy chain framework regions of the humanized antibodies or humanized antigen-binding fragments thereof have about 95% sequence identity to the framework regions of SEQ ID NO: 23 or 24.

Also disclosed are scFv and scFab fragments of the antibodies.

In some embodiments, the scFv can comprise a heavy chain variable region, a linker region, and a light chain variable region, wherein:
the heavy chain variable region comprises heavy chain CDR1, CDR2, and CDR3 of SEQ ID NOs: 8, 9, and 11, and the light chain variable region comprises light chain CDR1, CDR2, and CDR3 of SEQ ID NOs: 12, 14, and 15;
the heavy chain variable region comprises heavy chain CDR1, CDR2, and CDR3 of SEQ ID NOs: 7, 9, and 11, and the light chain variable region comprises light chain CDR1, CDR2, and CDR3 of SEQ ID NOs: 12, 14, and 15; or
the heavy chain variable region comprises heavy chain CDR1, CDR2, and CDR3 of SEQ ID NOs: 7, 10, and 11, and the light chain variable region comprises light chain CDR1, CDR2, and CDR3 of SEQ ID NOs: 13, 14, and 16.

In some embodiments, the scFv comprises a heavy chain variable region comprising SEQ ID NO: 1 and a light chain variable region comprising SEQ ID NO: 2. In some embodiments, the scFv comprises a heavy chain variable region comprising SEQ ID NO: 3 and a light chain variable region comprising SEQ ID NO: 4. In some embodiments, the scFv comprises a heavy chain variable region comprising SEQ ID NO: 5 and a light chain variable region comprising SEQ ID NO: 6.

In some embodiments, the heavy chain variable region, the light chain variable region, or both the heavy chain variable region and the light chain variable region of the scFv is humanized. In some embodiments, the humanized heavy chain variable region of the scFv comprises SEQ ID NO: 17 and the humanized light chain variable region of the scFv comprises SEQ ID NO: 18. In some embodiments, the heavy chain framework regions of the scFv have about 95% sequence identity to the framework regions of SEQ ID NO: 23 or SEQ ID NO: 24. In some embodiments, the scFv includes a linker region comprising SEQ ID NO: 25 or SEQ ID NO: 26.

The scFv can be fused to a transmembrane domain. The scFv can be fused to a transmembrane domain and a signal transduction domain. The scFv can be fused to a transmembrane domain and an immune-costimulatory domain. In some embodiments, any one of the disclosed scFvs can be fused to the co-stimulatory domain comprising the amino acid sequence of SEQ ID NO: 42 or 43.

The disclosed scFabs can comprise a heavy chain region, a linker region, and a light chain region, the light chain region comprising a light chain variable region and a light chain constant region, wherein
the heavy chain region comprises heavy chain CDR1, CDR2, and CDR3 of SEQ ID NOs: 8, 9, and 11, and the light chain variable region comprises light chain CDR1, CDR2, and CDR3 of SEQ ID NOs: 12, 14, and 15;
the heavy chain region comprises heavy chain CDR1, CDR2, and CDR3 of SEQ ID NOs: 7, 9, and 11, and the light chain variable region comprises light chain CDR1, CDR2, and CDR3 of SEQ ID NOs: 12, 14, and 15; or
the heavy chain region comprises heavy chain CDR1, CDR2, and CDR3 of SEQ ID NOs: 7, 10, and 11, and the light chain variable region comprises light chain CDR1, CDR2, and CDR3 of SEQ ID NOs: 13, 14, and 16.

In some embodiments, the scFab comprises a heavy chain region comprising SEQ ID NO: 1 and a light chain region comprising SEQ ID NO: 2. In some embodiments, the scFab comprises a heavy chain region comprising SEQ ID NO: 3 and a light chain region comprising SEQ ID NO: 4. In some embodiments, the scFab comprises a heavy chain region comprising SEQ ID NO: 5 and a light chain region comprising SEQ ID NO: 6.

In some embodiments, the heavy chain region, the light chain region, or both the heavy chain region and the light chain region of the scFab is humanized. In some embodiments, the humanized heavy chain region of the scFab comprises SEQ ID NO: 17 and the humanized light chain region of the scFab comprises SEQ ID NO: 18. In some embodiments, the heavy chain framework regions of the scFab have about 95% sequence identity to the framework regions of SEQ ID NO: 23 or SEQ ID NO: 24. In some embodiments, the scFab includes a linker region comprising SEQ ID NO: 25 or SEQ ID NO: 26.

Also disclosed are diagnostic and/or therapeutic compositions comprising any of the disclosed humanized F77 antibodies and their antigen-binding fragments.

Also disclosed are chimeric antigen receptors (CARs). The CARs can comprise the disclosed scFvs fused to the co-stimulatory domain comprising the amino acid sequence of SEQ ID NO: 42 or 43. The CAR can comprise an amino acid sequence of SEQ ID NO: 38, 39, 40, or 41. The CAR can be encoded by a nucleic acid molecule comprising SEQ ID NO: 34, 35, 36, or 37.

Also disclosed are lentiviral vectors comprising nucleic acid sequences encoding the disclosed CARs. Also disclosed are lentiviruses comprising the disclosed CARs.

Eukaryotic cells comprising nucleic acid sequences encoding the disclosed CARs are also provided. Eukaryotic cells comprising amino acid sequences of the disclosed CARs are also provided. In some embodiments, the eukaryotic cells are mammalian T cells comprising the disclosed CARs. In some embodiments, the mammalian T cell comprises an amino acid sequence of SEQ ID NO: 38, 39, 40, or 41.

The disclosed compositions and CARs can be used for diagnosis and/or treatment of localized (stages I and II), locally advanced (stage III), or advanced (stage IV) prostate cancer. Also provided are diagnostic or therapeutic compositions and CARs comprising clones of murine F77 monoclonal antibodies, humanized F77 antibodies, or antigen-binding fragments thereof.

### BRIEF DESCRIPTION OF THE DRAWINGS

The summary, as well as the following detailed description, is further understood when read in conjunction with the appended drawings. For the purpose of illustrating the disclosed antibodies and antigen-binding fragments thereof, exemplary embodiments of the antibodies and antigen-binding fragments thereof are shown in the drawings; however, the antibodies and antigen-binding fragments thereof are not limited to the specific embodiments disclosed. In the drawings:

FIG. 1A, FIG. 1B, FIG. 1C, and FIG. 1D are images showing the purification and crystallization of F77Fab (mouse monoclonal, clone F77 129.16) and its complex with GSC-915. FIG. 1A illustrates 12% SDS-PAGE assay of purified F77Fab (clone F77 129.16) Lane NR and R are the F77Fab sample under non-reduced and reduced conditions, respectively. Lane M is a protein molecular weight marker from ThermoFisher. The bands are proteins with molecular weights of 250 KD, 130 KD, 100 KD, 70 KD, 55 KD, 35 KD, 25 KD, and 15 KD from top to bottom. FIG. 1B illustrates crystals of the Fab fragment of F77 in 100 mM citrate sodium pH 5.6 and 12% PEG8K. FIG. 1C is an exemplary diffraction map of a crystal of the F77Fab. The crystal was flash moved to liquid nitrogen and the diffraction dataset was collected at 100K. FIG. 1D illustrates crystals of the F77Fab complexed with GSC-915 in 100 mM citrate sodium pH 5.6 and 12% PEG8K.

FIG. 2A is a diagram showing the structure model of the Fab fragment of mouse monoclonal F77 (mouse monoclonal). The complementarity determining regions are labeled L1, L2, L3, H1, H2, H3. FIG. 2B is a diagram showing a structure model for the F77 Fv (mouse monoclonal) and the sites of interest K3, Q75, and T83 in F77 heavy chain. The CDR regions of F77 heavy chain (H1, H2, and H3) are shown, and the three mutated residues (K3, Q75, and T83) are indicated.

FIG. 3A and FIG. 3B are graphs showing the results of an exemplary binding assay of the indicated antibodies to PC-3 cells. After the PC-3 cell's Fc receptors were blocked for 5 minutes with 5% FBS/PBS, the cells were incubated for 30 minutes with either (FIG. 3A) humanized F77N76S (labeled as N2S) or (FIG. 3B) mouse monoclonal F77 (clone: F77 129.16) from hybridoma. Primary antibody was diluted in 5% FBS/PBS to the indicated concentrations. Cells were washed twice with 1% BSA/PBS. Afterwards, cells were incubated for 30 minutes with anti-human IgG, Fc fragment conjugated to fluorescein (Jackson ImmunoResearch) (F77 in FIG. 3B is the mouse antibody, and anti-mouse IgG secondary antibody was used for FIG. 3B). Cells were then washed twice with 1% BSA/PBS, resuspended in FACS buffer, and analyzed using a BD LSR at 530 nm±15 nm. The shift of binding curve of F77N76S shows that the humanized antibody binds to PC-3.

FIG. 4A, FIG. 4B, FIG. 4C, FIG. 4D, FIG. 4E, FIG. 4F, FIG. 4I, and FIG. 4J are scatter plots showing the analysis of F77 scFv surface expression by flow cytometry using the CFP/GFP channel. All four F77 CART constructs have a CFP tag and show fluorescence in the CFP channel (450/50 violet). Fluorescence was also seen in the GFP channel (530/30 blue).

FIG. 5A, FIG. 5B, FIG. 5C, FIG. 5D, FIG. 5E, FIG. 5F, FIG. 5G, FIG. 5H, FIG. 5I, FIG. 5J, FIG. 5K, FIG. 5L, FIG. 5M, FIG. 5N, and FIG. 5O are scatterplots showing the analysis of F77 scFv surface expression by flow cytometry.

FIG. 6A, FIG. 6B, and FIG. 6C are scatterplots showing the analysis of EGFR VIII expression by flow cytometry.

FIG. 7 is a bar graph showing a Human IFN-gamma ELISA assay.

FIG. 8A, FIG. 8B, FIG. 8C, FIG. 8D, and FIG. 8E are graphs showing impedance monitoring of PC3 cells using xCELLigence RTCA system and % Cytolysis measurement after addition of different effector cells at varying E:T ratios. Impedance monitoring was validated using T cells without any CAR (NTD; FIG. 8A), F77 VLVH 4-1BBzeta (FIG. 8B), F77 VHVL CD28zeta (FIG. 8C) and 2173 CD28zeta (EGFRvIII CD28zeta, FIG. 8D) effector cells over GFP labeled prostate cancer cells PC3 at E:T ratios of 15:1 (circle-annotated line), 10:1 (square-annotated line) and 5:1 (triangle-annotated line). When seeded alone, target cells adhered to the plate and proliferated, increasing Cell Index (CI) readout (diamond-annotated line). When added to target cells, F77 effector cells caused progressive decrease in CI values at all E:T ratios leading to complete cytolysis of target cells. Y-axis was normalized Cell Index (NCI) generated by RTCA software and displayed in real time. X-axis was the time of cell culture and treatment time in hour. Mean values of CI were plotted ±SEM. NCI values have been used to calculate % Cytolysis using formula described in materials and methods. (FIG. 8E) % Cytolysis for PC3 cells has been calculated at 40 hr (16 hr after addition of effector cells, T=16 hr).

FIG. 9A, FIG. 9B, FIG. 9C, FIG. 9D, and FIG. 9E are graphs showing impedance monitoring of PC3 FUT1KO cells using xCELLigence RTCA system and % Cytolysis measurement after addition of different effector cells at varying E:T ratios. Impedance monitoring was validated using T cells without any CAR (NTD; FIG. 9A), F77 VLVH 4-1BBzeta (FIG. 9B), F77 VHVL CD28zeta (FIG. 9C) and 2173 CD28zeta (EGFRvIII CD28zeta, FIG. 9D) effector cells over GFP labeled prostate cancer cells PC3 FUT1KO at E:T ratios of 15:1 (circle-annotated line), 10:1 (square-annotated line) and 5:1 (triangle-annotated line). When seeded alone, target cells adhered to the plate and proliferated, increasing Cell Index (CI) readout (diamond-annotated line). When added to target cells, F77 effector cells caused minimal or no decrease in CI values at all E:T ratios leading to non-significant cytolysis of target cells. Y-axis was normalized Cell Index (NCI) generated by RTCA software and displayed in real time. X-axis was the time of cell culture and treatment time in hour. Mean values of CI were plotted ±SEM. NCI values have been used to calculate % Cytolysis using formula described in materials and methods. (FIG. 9E) % Cytolysis for PC3 FUT1KO cells has been calculated at 40 hr (16 hr after addition of effector cells, T=16 hr). % Cytolysis calculated for PC3 FUT1KO cells after addition of F77 effector cells is not significant.

FIG. 10A, FIG. 10B, FIG. 10C, FIG. 10D, and FIG. 10E are graphs showing impedance monitoring of normal prostate epithelial cells RWPE-1 using xCELLigence RTCA system and % Cytolysis measurement after addition of different effector cells at varying E:T ratios. Impedance monitoring was validated using T cells without any CAR (NTD; FIG. 10A), F77 VLVH 4-1BBzeta (FIG. 10B), F77 VHVL CD28zeta (FIG. 10C) and 2173 CD28zeta (EGFRvIII CD28zeta, FIG. 10D) effector cells over GFP labeled RWPE-1 cells at E:T ratios of 15:1 (circle-annotated line), 10:1 (square-annotated line) and 5:1 (triangle-annotated line). When seeded alone, target cells adhered to the plate and proliferated, increasing Cell Index (CI) readout (diamond-annotated line). When added to target cells, F77 effector cells caused minimal or no decrease in CI values at all E:T ratios leading to incomplete cytolysis of target cells. Y-axis was normalized Cell Index (NCI) generated by RTCA software and displayed in real time. X-axis was the time of cell culture and treatment time in hour. Mean values of CI were plotted ±SEM. NCI values have been used to calculate % Cytolysis using formula described in materials and methods. (FIG. 10E) % Cytolysis for RWPE-1 cells has been calculated at 40 hr (16 hr after addition of effector cells, T=16 hr). Some level of % Cytolysis is calculated for RWPE-1 cells after addition of F77 effector cells which is attributed to the limited expression of F77 antigen on these cells as shown by binding with mAb F77 55.10 using flow cytometry.

FIG. 11A, FIG. 11B, FIG. 11C, FIG. 11D, and FIG. 11E are graphs showing impedance monitoring of tumorigenic prostate epithelial cells RWPE-2 cells using xCELLigence RTCA system and % Cytolysis measurement after addition of different effector cells at varying E:T ratios. Impedance monitoring was validated using T cells without any CAR (NTD; FIG. 11A), F77 VLVH 4-1BBzeta (FIG. 11B), F77 VHVL CD28zeta (FIG. 11C) and 2173 CD28zeta (EGFRvIII CD28zeta, FIG. 11D) effector cells over GFP labeled RWPE-2 prostate cancer cells at E:T ratios of 15:1 (circle-annotated line), 10:1 (square-annotated line) and 5:1 (triangle-annotated line). When seeded alone, target cells adhered to the plate and proliferated, increasing Cell Index (CI) readout (diamond-annotated line). When added to target cells, F77 effector cells caused an immediate drop in CI values at all E:T ratios leading to complete cytolysis of target cells. Y-axis was normalized Cell Index (NCI) generated by RTCA software and displayed in real time. X-axis was the time of cell culture and treatment time in hour. Mean values of CI were plotted ±SEM. NCI values have been used to calculate % Cytolysis using formula described in materials and methods. (FIG. 11E) % Cytolysis for RWPE-2 cells has been calculated at 40 hr (16 hr after addition of effector cells, T=16 hr).

FIG. 12 is a graph showing percent cytolysis (% Cytolysis) for U87 GFP cells with corresponding numerical values at different Effector (E) to Target (T) cell ratio (E:T ratio) from killing assay using eSight xCELLigence.

FIG. 13A, FIG. 13B, FIG. 13C, FIG. 13D, and FIG. 13E are graphs showing impedance monitoring of U87EGFRvIII cells using xCELLigence RTCA system and % Cytolysis measurement after addition of different effector cells at varying E:T ratios. Impedance monitoring is validated using T cells without any CAR (NTD; FIG. 13A), F77 VLVH 4-1BBzeta (FIG. 13B), F77 VHVL CD28zeta (FIG. 13C) and 2173 CD28zeta (EGFRvIII CD28zeta, FIG. 13D) effector cells over GFP labeled U87EGFRvIII cells (express EGFRvIII) at E:T ratios of 15:1 (circle-annotated line), 10:1 (square-annotated line) and 5:1 (triangle-annotated line). When seeded alone, target cells adhered to the plate and proliferated, increasing Cell Index (CI) readout (diamond-annotated line). When added to target cells, 2173 CD28zeta effector cells caused progressive decrease in CI values at all E:T ratios leading to complete cytolysis of target cells. Y-axis was normalized Cell Index (NCI) generated by RTCA software and displayed in real time. X-axis was the time of cell culture and treatment time in hour. Mean values of CI were plotted ±SEM. NCI values have been used to calculate % Cytolysis using formula described in materials and methods. (FIG. 13E) % Cytolysis for U87EGFRvIII cells has been calculated at 40 hr (16 hr after addition of effector cells, T=16 hr). No significant decrease in CI values and thus no cytolysis is seen in U87EGFRvIII cells after addition of F77 CAR T cells.

FIG. 14 is a graph showing that treatment of mice bearing PC3 subcutaneous tumors with F77 effector cells lead to tumor regression. NSG mice were injected subcutaneously with 5×10⁶ PC3 cells. Fourteen days later the mice received intratumoral injection of 5×10⁶ NTD (T-cells without any CAR) or F77 VLVH 4-1BBzeta or F77 VHVL CD28zeta CAR T cells. Five mice per group were used and tumor volumes were measured by caliper twice per week. Administration of F77 CAR T cells led to complete regression of PC3 tumors in the mouse model tested.

FIG. 15 is a schematic representation of F77 CAR construct design. From N-terminus to the C-terminus, all F77 CARs included cyan fluorescent protein (CFP) tag, porcine teschovirus-1 (P2A) peptide and F77 scFv in VH to VL or VL to VH orientation upstream of CD8a hinge and transmembrane domain (CD8 TM or CD28 TM), followed by 4-1BB or CD28 intracellular domain (ICD) and CD3 zeta (CD3-ζ) intracellular signaling domains.

FIG. 16A, FIG. 16B, FIG. 16C, and FIG. 16D are graphs showing the surface expression of F77 antigen on prostate cancer cells using mAb F77 55.10. Flow cytometry was performed to compare the surface expression of F77 antigen among PC3, RWPE-1, RWPE-2 and PC3 FUT1 KO cell lines. The surface expression of F77 is shown by the right-most bell-shaped curve. Benign prostate epithelial cells RWPE-1 showed minimal F77 positivity over PC3 cells and tumorigenic prostate epithelial cells RWPE-2. Knockout of FUT1 dramatically decreased mAb F77 55.10 binding in PC3 FUT1KO cells.

FIG. 17A and FIG. 17B are graphs depicting surface expression of F77 antigen on prostate cancer cells using mAb F77 55.10. FIG. 17A—Flow cytometry was performed to compare the surface expression of F77 antigen among PC3, PC3 FUT1KO, RWPE-1 and RWPE-2 cells. Benign prostate epithelial cells RWPE-1 showed minimal F77 positivity over PC3 cells and tumorigenic prostate epithelial cells RWPE-2. Knockout of FUT1 dramatically decreased mAb F77 55.10 binding to PC3 FUT1KO cells. Peaks on the right depict the staining results obtained using monoclonal antibody F77 55.10, while the peaks on the left correspond to the control staining, where target cells were labeled with FITC-labeled goat anti-mouse IgG-Fc7 antibody. FIG. 17B—Mean fluorescence intensities of different prostate cell-lines after staining with mAb F77 55.10.

FIG. 18A and FIG. 18B are graphs depicting comparative analysis of surface expression of F77 antigen on PC3 prostate cancer cells in relation to glioblastoma cells U87 and U87EGFRvIII utilizing mAb F77 55.10. FIG. 18A—Flow cytometry was performed to compare the surface expression of F77 antigen among PC3, U87 and U87EGFRvIII. Peaks on the right represent the staining by mAb F77 55.10 while the peaks on the left represent the control staining (target cells labeled with APC-labeled goat anti-mouse antibody). FIG. 18B—Mean fluorescence intensities of different cell lines after staining with mAb F77 55.10.

FIG. 19 is a schematic representation of F77 CAR construct design. From N-terminus to the C-terminus, both F77 CARs included cyan fluorescent protein (CFP) tag, porcine teschovirus-1 (P2A) peptide and F77 scFv in VL to VH or VH to VL orientation upstream of CD8a hinge and transmembrane domain (CD8 TM or CD28TM), followed by 4-1BB or CD28 and CD3 zeta (CD3-ζ) intracellular signaling domains. F77 VLVH 4-1BBzeta and F77 VHVL CD28zeta have been designated as F77 CAR1 and F77 CAR2, respectively.

FIG. 20A and FIG. 20B are graphs depicting surface expression of F77 CAR on T cells. Primary human T cells were activated using anti-CD3/anti-CD28 coated Dynabeads™ followed by transduction with lentiviral vectors encoding indicated CARs or were left non-transduced (NTD). Eight days later, the CFP expression of the F77 CAR molecule on the surface of T cells was detected with the (FIG. 20A) violet laser (440/40 nm) and the expression of the Control CAR was detected with the blue laser (530/30 nm) after staining with Alexa Fluor 488 AffiniPure Goat Anti Human IgG F(ab′₂) and analyzed by flow cytometry (LSR FortessaC, BD). FIG. 20B—the graph represents the percentage of expression in cells from three independent experiments.

FIG. 21A, FIG. 21B, FIG. 21C, FIG. 21D, and FIG. 21E are graphs depicting IFNγ ELISA assay. Secretion levels of IFN-γ by CAR-T cells co-cultured with (FIG. 21A) PC3, (FIG. 21B) PC3 FUT1KO, (FIG. 21C) RWPE-1, (FIG. 21D) RWPE-2 or (FIG. 21E) U87EGFRvIII cells. Graph bars present the mean cytokine concentration (pg/ml)±SEM from duplicate wells from two independent experiments. Statistical analyses were performed using one-way ANOVA with Tukey post hoc correction test.

FIG. 22A, FIG. 22B, FIG. 22C, FIG. 22D, and FIG. 22E are graphs depicting impedance monitoring of PC3 cells using xCELLigence RTCA system and percent (%) Cytolysis measurement after addition of different effector cells at varying E:T ratios. Impedance monitoring was validated using T cells without any CAR (NTD; FIG. 22A), F77 CAR1 (FIG. 22B), F77 CAR2 (FIG. 22C) and control CAR (FIG. 22D) effector cells over GFP labeled prostate cancer cells PC3 at E:T ratios of 15:1, 10:1, and 5:1. When seeded alone, target cells adhered to the plate and proliferate, increasing Cell Index (CI) readout (line with diamond symbols). When added to target cells, F77 effector cells caused progressive decrease in CI values at all E:T ratios leading to complete cytolysis of target cells. Y-axis is normalized Cell Index (NCI) generated by RTCA software and displayed in real time. X-axis is the time of cell culture and treatment time in hour. Mean values of CI were plotted ±SEM. NCI values were used to calculate % cytolysis using formula described in Materials and Methods. (FIG. 22E) % Cytolysis for PC3 cells was calculated at 40 h (16 h after addition of effector cells, T=16 h).

FIG. 23A, FIG. 23B, FIG. 23C, FIG. 23D, and FIG. 23E are graphs depicting impedance monitoring of PC3 FUT1KO cells using xCELLigence RTCA system and percent (%) Cytolysis measurement after addition of different effector cells at varying E:T ratios. Impedance monitoring is validated using T cells without any CAR (NTD; FIG. 23A), F77 CAR1 (FIG. 23B), F77 CAR2 (FIG. 23C) and control CAR (FIG. 23D) effector cells over GFP labeled prostate cancer cells PC3 FUT1KO at E:T ratios of 15:1, 10:1, and 5:1. When seeded alone, target cells adhered to the plate and proliferate, increasing Cell Index (CI) readout (line with diamond symbols). When added to target cells, F77 effector cells caused minimal or no decrease in CI values at all E:T ratios leading to non-significant cytolysis of target cells. Y-axis is normalized Cell Index (NCI) generated by RTCA software and displayed in real time. X-axis is the time of cell culture and treatment time in hour. Mean values of CI were plotted ±SEM. NCI values were used to calculate % cytolysis using formula described in Materials and Methods. (FIG. 23E) % Cytolysis for PC3 FUT1KO cells was calculated at 40 h (16 h after addition of effector cells, T=16 h). Addition of F77 effector cells to PC3 FUT1KO cells does not induce any markedly noticeable % cytolysis.

FIG. 24A, FIG. 24B, FIG. 24C, FIG. 24D, and FIG. 24E are graphs depicting impedance monitoring of prostate epithelial cell line RWPE-1 cells using xCELLigence RTCA system and percent (%) Cytolysis measurement after addition of different effector cells at varying E:T ratios. Impedance monitoring is validated using T cells without any CAR (NTD; FIG. 24A), F77 CAR1 (FIG. 24B), F77 CAR2 (FIG. 24C) and control CAR (FIG. 24D) effector cells over GFP labeled RWPE-1 cells at E:T ratios of 15:1, 10:1, and 5:1. When seeded alone, target cells adhered to the plate and proliferate, increasing Cell Index (CI) readout (line with diamond symbols). When added to target cells, F77 effector cells caused minimal or no decrease in CI values at all E:T ratios leading to incomplete cytolysis of target cells. Y-axis is normalized Cell Index (NCI) generated by RTCA software and displayed in real time. X-axis is the time of cell culture and treatment time in hour. Mean values of CI were plotted ±SEM. NCI values were used to calculate % cytolysis using formula described in Materials and Methods. (FIG. 24E) % Cytolysis for RWPE-1 cells was calculated at 40 h (16 h after addition of effector cells, T=16 h). Some level of % cytolysis was observed for RWPE-1 cells after addition of F77 effector cells which is attributed to the minimal F77 positivity shown by these target cells shown by binding with mAb F77 55.10 using flow cytometry.

FIG. 25A, FIG. 25B, FIG. 25C, FIG. 25D, and FIG. 25E are graphs depicting impedance monitoring of tumorigenic prostate epithelial cell line RWPE-2 cells using xCELLigence RTCA system and percent (%) Cytolysis measurement after addition of different effector cells at varying E:T ratios. Impedance monitoring is validated using T cells without any CAR (NTD; FIG. 25A), F77 CAR1 (FIG. 25B), F77 CAR2 (FIG. 25C) and control CAR (FIG. 25D) effector cells over GFP labeled RWPE-2 prostate cancer cells at E:T ratios of 15:1, 10:1, and 5:1. When seeded alone, target cells adhered to the plate and proliferate, increasing Cell Index (CI) readout (line with diamond symbols). When added to target cells, F77 effector cells caused immediate drop in CI values at all E:T ratios leading to complete cytolysis of target cells. Y-axis is normalized Cell Index (NCI) generated by RTCA software and displayed in real time. X-axis is the time of cell culture and treatment time in hour. Mean values of CI were plotted ±SEM. NCI values were used to calculate % cytolysis using formula described in Materials and Methods. (FIG. 25E) % Cytolysis for RWPE-2 cells was calculated at 40 h (16 h after addition of effector cells, T=16 h)

FIG. 26A, FIG. 26B, FIG. 26C, FIG. 26D, and FIG. 26E are graphs depicting impedance monitoring of U87EGFRvIII cells using xCELLigence RTCA system and percent (%) Cytolysis measurement after addition of different effector cells at varying E:T ratios. Impedance monitoring is validated using T cells without any CAR (NTD; FIG. 26A), F77 CAR1 (FIG. 26B), F77 CAR2 (FIG. 26C) and control CAR (FIG. 26D) effector cells over GFP labeled U87EGFRvIII cells (express mutant EGFR) at E:T ratios of 15:1, 10:1, and 5:1. When seeded alone, target cells adhered to the plate and proliferate, increasing Cell Index (CI) readout (line with diamond symbols). When added to target cells, control CAR effector cells caused progressive decrease in CI values at all E:T ratios leading to complete cytolysis of target cells. Y-axis is normalized Cell Index (NCI) generated by RTCA software and displayed in real time. X-axis is the time of cell culture and treatment time in hour. Mean values of CI were plotted ±SEM. NCI values were used to calculate % cytolysis using formula described in Materials and Methods. (FIG. 26E) % Cytolysis for U87EGFRvIII cells was calculated at 40 h (16 h after addition of effector cells, T=16 h). No noticeable decrease in CI values and thus no cytolysis was seen in U87EGFRvIII cells after addition of F77 effector cells.

FIG. 27A, FIG. 27B, FIG. 27C, FIG. 27D, and FIG. 27E are graphs depicting percent (%) cytolysis determined through xCELLigence impedance measurement at 5:1 E:T ratio of CAR T cells and cancer cells. % Cytolysis for cancer cells was calculated after 24 h of cancer cell addition (FIG. 27A) PC3, (FIG. 27A B) PC3 FUT1KO, (FIG. 27A C) RWPE-1, (FIG. 27A D) RWPE-2, (FIG. 27A E) U87EGFRvIII and every 5 h after effector cells addition. Impedance monitoring was validated using T cells without any CAR (NTD), F77 CAR1, F77 CAR2 and control CAR effector cells over GFP labeled target cells at E:T ratios of 5:1. Normalized Cell Index (NCI) was used to calculate % Cytolysis and plotted ±SEM using the formula described in Materials and Methods. Significance of difference in activity of each CAR compared to NTD was determined using One-way ANOVA followed by Dunnett's correction for multiple comparisons. Asterisks indicate significance (** p<0.0001 and ns refers to p>0.05).

FIG. 28 is a set of graphs depicting EGFR surface expression on prostate cancer cells using mAb EGFR 528 (sc-120). Flow cytometry was performed to compare the surface expression of EGFR among PC3, PC3 FUT1KO, RWPE-1, RWPE-2 and U87 cells using PE conjugated mouse mAb EGFR 528 (sc-120). The peaks on the right represent the staining by mAb EGFR 528 (sc-120), while the peaks on the left denote the target cells in the absence of any antibody.

FIG. 29A, FIG. 29B, and FIG. 29C are a diagram (FIG. 29A) and graphs depicting treatment of mice bearing PC3 subcutaneous tumors with F77 effector cells leads to tumor regression. (FIG. 29A) Schematic representation of the experimental strategy. NSG mice were injected subcutaneously with 5×10⁶ PC3 cells. Fourteen days later the mice received intratumoral injection of 5×10⁶ NTD (T-cells without any CAR) or F77 CAR1 or F77 CAR2 T cells and tumor volumes were measured by caliper twice per week. (FIG. 29B) Tumor volume graph shows that administration of F77 CAR T cells led to complete regression of PC3 tumors in the mouse model tested (n=5). (FIG. 29C) Data on tumor volume at day 27 for F77 CAR1 treated mice shows significant difference in tumor volume leading to complete remission in comparison to NTD treated mice. Asterisks indicate significance (** p<0.001 and ns refers to p>0.05, using Kruskal-Wallis test followed by Dunnett's multiple comparisons test).

### DETAILED DESCRIPTION

The disclosed clones of murine F77 monoclonal antibody, humanized F77 antibodies, as well as antigen-binding fragments thereof may be understood more readily by reference to the following detailed description taken in connection with the accompanying figures, which form a part of this disclosure. It is to be understood that the disclosed clones of murine F77 monoclonal antibody, humanized F77 antibodies, as well as antigen-binding fragments thereof are not limited to the specific clones of murine F77 monoclonal antibody, humanized F77 antibodies, as well as antigen-binding fragments thereof described and/or shown herein, and that the terminology used herein is for the purpose of describing particular embodiments by way of example only and is not intended to be limiting of the claimed clones of murine F77 monoclonal antibody, humanized F77 antibodies, as well as antigen-binding fragments thereof.

Unless specifically stated otherwise, any description as to a possible mechanism or mode of action or reason for improvement is meant to be illustrative only, and the disclosed clones of murine F77 monoclonal antibody, humanized F77 antibodies, as well as antigen-binding fragments thereof are not to be constrained by the correctness or incorrectness of any such suggested mechanism or mode of action or reason for improvement.

Throughout this text, the descriptions refer to antibodies and methods of using said antibodies. Where the disclosure describes or claims a feature or embodiment associated with an antibody, such a feature or embodiment is equally applicable to the methods of using said antibody. Likewise, where the disclosure describes or claims a feature or embodiment associated with a method of using an antibody, such a feature or embodiment is equally applicable to the antibody.

When values are expressed as approximations, by use of the antecedent “about,” it will be understood that the particular value forms another embodiment. Reference to a particular numerical value includes at least that particular value, unless the context clearly dictates otherwise.

It is to be appreciated that certain features of the disclosed antibodies and methods which are, for clarity, described herein in the context of separate embodiments, may also be provided in combination in a single embodiment. Conversely, various features of the disclosed antibodies and methods that are, for brevity, described in the context of a single embodiment, may also be provided separately or in any subcombination.

Various terms relating to aspects of the description are used throughout the specification and claims. Such terms are to be given their ordinary meaning in the art unless otherwise indicated. Other specifically defined terms are to be construed in a manner consistent with the definitions provided herein.

As used in this specification and the appended claims, the singular forms “a,” “an,” and “the” include plural referents unless the content clearly dictates otherwise. Thus, for example, reference to “a cell” includes a combination of two or more cells, and the like.

The term “about” as used herein when referring to a measurable value such as an amount, a temporal duration, and the like, is meant to encompass variations of ±10%, ±5%, ±1% or ±0.1% from the specified value, as such variations are appropriate to perform the disclosed methods.

“Substantially similar” with respect to nucleic acid or amino acid sequences means at least about 65% sequence identity between two or more sequences. Substantially similar can include at least about 70% sequence identity between two or more sequences, at least about 75% sequence identity, at least about 80% sequence identity, at least about 85% sequence identity, at least about 90% sequence identity, at least about 91% sequence identity, at least about 92% sequence identity, at least about 93% sequence identity, at least about 94% sequence identity, at least about 95% sequence identity, at least about 96% sequence identity, at least about 97% sequence identity, at least about 98% sequence identity, or at least about 99% or greater sequence identity. Such identity can be determined using algorithms known in the art, such as the mBLAST algorithm.

“Antibody” refers to all isotypes of immunoglobulins (IgG, IgA, IgE, IgM, IgD, and IgY) including various monomeric and polymeric forms of each isotype, unless otherwise specified.

“Antigen-binding fragments” of antibodies refer to portions of intact antibodies that retain antigen-binding specificity (binding affinity) of the parent antibody molecule. For example, antigen-binding fragments can comprise at least the CDRs of either the heavy chain or light chain variable region. Antigen-binding fragments can also comprise the heavy chain or light chain variable region, or sequences that are substantially similar to the heavy or light chain variable region. Further suitable antigen-binding fragments include, without limitation, antibodies with multiple epitope specificity, bispecific antibodies, diabodies, and single-chain molecules, as well as fragment antigen-binding (Fab), F(ab′)2, Fd, Fabc, and variable fragment (Fv) molecules, single chain (Sc) antibodies, single-chain variable fragment (scFv), single chain Fab (scFab), individual antibody light chains, individual antibody heavy chains, chimeric fusions between antibody chains and other molecules, heavy chain monomers or dimers, light chain monomers or dimers, dimers consisting of one heavy and one light chain, and the like. All antibody isotypes can be used to produce antigen-binding fragments. Antigen-binding fragments can be recombinantly or synthetically produced, with natural or unnatural nucleic acid or amino acid molecules (Bates and Power, Antibodies, 8, 28:1-31 (2019)).

The antibodies or antigen-binding fragments thereof can be generated from any species. The antibodies or antigen-binding fragments thereof can be labeled or otherwise conjugated to various chemical or biomolecule moieties, for example, for therapeutic or diagnostic use in detection or treatment applications. The moieties can be cytotoxic, for example, bacterial toxins, viral toxins, radioisotopes, and the like. The moieties can be detectable labels, for example, fluorescent labels, radiolabels, biotin, and the like, which are known in the art.

The terms “treating” or “treatment” refer to any success or indicia of success in the attenuation or amelioration of a prostate cancer, including any objective or subjective parameter such as abatement, remission, diminishing of symptoms or making the injury, pathology, or condition more tolerable to the subject, improving the subject's physical well-being, or prolonging the length of survival. The treatment or amelioration of symptoms can be based on objective or subjective parameters; including the results of a physical examination, laboratory test(s), non-invasive imaging test(s), and/or self-reporting by the subject.

“Effective amount” and “therapeutically effective amount” are used interchangeably herein, and refer to an amount of an antibody or an antigen-binding fragment thereof effective to achieve a particular biological or therapeutic result such as, but not limited to, amelioration of one or more symptoms of prostate cancer. A therapeutically effective amount of the antibody or antigen-binding fragment thereof may vary according to factors such as the disease state, age, sex, and weight of the individual, and the ability of the antibody or the antigen-binding fragment thereof to elicit a desired response in the individual. Such results may include, but are not limited to, the treatment of prostate cancer, as determined by any means suitable in the art.

### F77 Antibodies and Fragments Thereof

The disclosed antibodies and their fragments bind to a glycolipid-like antigen Prostate Cancer Lipid-like Antigen (PCLA) that is highly restricted to the prostate cancer cell or prostasome surfaces. The PC3 cell-specific mAb F77 (Carroll A M, et al. Clin Immunol Immunopathol 33:268-281 (1984)) recognizes a unique glycolipid antigen highly restricted to the prostate cancer cell surface termed prostate cancer lipid antigen (PCLA). The unique binding pattern of mAb F77 indicates that PCLA exists predominantly in prostate and that its expression is consistently higher in tumor tissues than in normal tissues. Because PCLA remains expressed on both androgen-dependent and androgen-independent prostate cancer, it may be exploited as a target for diagnosis and treatment of both early and late stages of the disease.

The disclosed antibodies include clones of the murine F77 monoclonal antibody and humanized antibodies derived from clones of the murine F77 monoclonal antibody (referred to herein as “humanized F77 antibodies”). The variable fragment (Fv) containing the CDRs of the heavy chain and the CDRs of the light chain may be derived from the clones of the murine F77 monoclonal antibody, from mutated clones of the murine F77 monoclonal antibody, from humanized antibodies, from mutated humanized antibodies, or represent a consensus Fv sequence between murine heavy Fv and human heavy Fv sequences, or a consensus Fv sequence between murine light Fv and human light Fv sequences.

The constant region (Fc (Fragment, crystallizable)) of the heavy and/or the light chain may be derived from murine antibodies, from mutated murine antibodies, from human antibodies, from mutated human antibodies, or represent a consensus Fc sequence between murine heavy Fc and human heavy Fc sequences, or a consensus Fc sequence between murine light Fc and human light Fc sequences.

scFv and scFab derived from clones of the murine F77 monoclonal antibody or from humanized F77 antibodies are described.

Provided herein are clones of the murine F77 monoclonal antibody. The disclosed clones include murine F77 monoclonal antibody with clone ID 129.5, murine F77 monoclonal antibody with clone ID 129.16, and murine F77 monoclonal antibody with clone ID 55.10.

The murine F77 monoclonal antibody clone ID 129.5 comprises a heavy chain variable region having an amino acid sequence as set forth in SEQ ID NO: 1 and a light chain variable region having an amino acid sequence as set forth in SEQ ID NO: 2.

The murine F77 monoclonal antibody clone ID 129.16 comprises a heavy chain variable region having an amino acid sequence as set forth in SEQ ID NO: 3 and a light chain variable region having an amino acid sequence as set forth in SEQ ID NO: 4.

The murine F77 monoclonal antibody clone ID 55.10 comprises a heavy chain variable region having an amino acid sequence as set forth in SEQ ID NO: 5 and a light chain variable region having an amino acid sequence as set forth in SEQ ID NO: 6.

Also provided are antigen-binding fragments of the murine F77 monoclonal antibody clones with clone ID 129.5, clone ID 129.16, and clone ID 55.10. The antigen-binding fragments of the murine F77 monoclonal antibody clones include scFv and scFab, where
the heavy chain variable region comprises heavy chain CDR1, CDR2, and CDR3 of SEQ ID NOs: 7, 9, and 11, and the light chain variable region comprises light chain CDR1, CDR2, and CDR3 of SEQ ID NOs: 12, 14, and 15; or
the heavy chain variable region comprises heavy chain CDR1, CDR2, and CDR3 of SEQ ID NOs: 7, 10, and 11, and the light chain variable region comprises light chain CDR1, CDR2, and CDR3 of SEQ ID NOs: 13, 14, and 16.

### Humanized F77 Antibodies and Fragments Thereof

Disclosed herein are humanized antibodies, humanized antigen-binding fragments, and compositions containing the humanized antibodies or humanized antigen-binding fragments. In some embodiments, the humanized antigen-binding fragments are scFvs or scFabs.

The humanized antibodies or the humanized antigen-binding fragments thereof can comprise a heavy chain variable region comprising heavy chain CDR1, CDR2, and CDR3 of SEQ ID NOs: 8, 9, and 11, and a light chain variable region comprising light chain CDR1, CDR2, and CDR3 of SEQ ID NOs: 12, 14, and 15.

The humanized antibodies or the humanized antigen-binding fragments thereof can comprise a heavy chain variable region comprising heavy chain CDR1, CDR2, and CDR3 of SEQ ID NOs: 7, 9, and 11, and a light chain variable region comprising light chain CDR1, CDR2, and CDR3 of SEQ ID NOs: 12, 14, and 15.

The humanized antibodies or the humanized antigen-binding fragments thereof can comprise a heavy chain variable region comprising heavy chain CDR1, CDR2, and CDR3 of SEQ ID NOs: 7, 10, and 11, and a light chain variable region comprising light chain CDR1, CDR2, and CDR3 of SEQ ID NOs: 13, 14, and 16.

In some embodiments, the heavy chain variable region of the humanized antibodies, or the humanized antigen-binding fragments, has framework regions having about 95% sequence identity to the framework regions of SEQ ID NO: 17. In some embodiments, the heavy chain variable region of the humanized antibodies, or the humanized antigen-binding fragments, has framework regions having about 99% sequence identity to the framework regions of SEQ ID NO: 17. In some embodiments, the heavy chain variable region of the humanized antibodies, or the humanized antigen-binding fragments, has framework regions having 100% sequence identity to the framework regions of SEQ ID NO: 17.

In some embodiments, the light chain variable region of the humanized antibodies, or the humanized antigen-binding fragments, has framework regions having about 95% sequence identity to the framework regions of SEQ ID NO: 18. In some embodiments, the light chain variable region of the humanized antibodies, or the humanized antigen-binding fragments, has framework regions having about 99% sequence identity to the framework regions of SEQ ID NO: 18. In some embodiments, the light chain variable region of the humanized antibodies, or the humanized antigen-binding fragments, has framework regions having 100% sequence identity to the framework regions of SEQ ID NO: 18.

In some embodiments, the heavy chain framework regions of the humanized antibodies or humanized antigen-binding fragments thereof have about 95% sequence identity to the framework regions of SEQ ID NO: 23. In some embodiments, the heavy chain framework regions of the humanized antibodies or humanized antigen-binding fragments thereof have about 99% sequence identity to the framework regions of SEQ ID NO: 23. In some embodiments, the heavy chain framework regions of the humanized antibodies or humanized antigen-binding fragments thereof have 100% sequence identity to the framework regions of SEQ ID NO: 23.

In some embodiments, the heavy chain framework regions of the humanized antibodies or humanized antigen-binding fragments thereof have about 95% sequence identity to the framework regions of SEQ ID NO: 24. In some embodiments, the heavy chain framework regions of the humanized antibodies or humanized antigen-binding fragments thereof have about 99% sequence identity to the framework regions of SEQ ID NO: 24. In some embodiments, the heavy chain framework regions of the humanized antibodies or humanized antigen-binding fragments thereof have 100% sequence identity to the framework regions of SEQ ID NO: 24.

In some embodiments, the humanized antibodies, or the humanized antigen-binding fragments, have heavy chain framework regions having about 95% sequence identity to the framework regions of SEQ ID NO: 17 and light chain framework regions having about 95% sequence identity to the framework regions of SEQ ID NO: 18. In some embodiments, the humanized antibodies, or the humanized antigen-binding fragments, have heavy chain framework regions having about 99% sequence identity to the framework regions of SEQ ID NO: 17 and light chain framework regions having about 99% sequence identity to the framework regions of SEQ ID NO: 18. In some embodiments, the humanized antibodies, or the humanized antigen-binding fragments, have heavy chain framework regions having 100% sequence identity to the framework regions of SEQ ID NO: 17 and light chain framework regions having 100% sequence identity to the framework regions of SEQ ID NO: 18.

In some embodiments, the humanized antibodies, or the humanized antigen-binding fragments, have heavy chain framework regions having about 95% sequence identity to the framework regions of SEQ ID NO: 23 and light chain framework regions having about 95% sequence identity to the framework regions of SEQ ID NO: 18. In some embodiments, the humanized antibodies, or the humanized antigen-binding fragments, have heavy chain framework regions having about 99% sequence identity to the framework regions of SEQ ID NO: 23 and light chain framework regions having about 99% sequence identity to the framework regions of SEQ ID NO: 18. In some embodiments, the humanized antibodies, or the humanized antigen-binding fragments, have heavy chain framework regions having 100% sequence identity to the framework regions of SEQ ID NO: 23 and light chain framework regions having 100% sequence identity to the framework regions of SEQ ID NO: 18.

In some embodiments, the humanized antibodies, or the humanized antigen-binding fragments, have heavy chain framework regions having about 95% sequence identity to the framework regions of SEQ ID NO: 24 and light chain framework regions having about 95% sequence identity to the framework regions of SEQ ID NO: 18. In some embodiments, the humanized antibodies, or the humanized antigen-binding fragments, have heavy chain framework regions having about 99% sequence identity to the framework regions of SEQ ID NO: 24 and light chain framework regions having about 99% sequence identity to the framework regions of SEQ ID NO: 18. In some embodiments, the humanized antibodies, or the humanized antigen-binding fragments, have heavy chain framework regions having 100% sequence identity to the framework regions of SEQ ID NO: 24 and light chain framework regions having 100% sequence identity to the framework regions of SEQ ID NO: 18.

Exemplary humanized antibodies include antibodies with a heavy chain amino acid sequence as in SEQ ID NO: 19 and a light chain amino acid sequence as in SEQ ID NO: 21.

Exemplary humanized antigen-binding fragments include fragments with a heavy chain variable region having an amino acid sequence as in SEQ ID NO: 17 and a light chain variable region having an amino acid sequence as in SEQ ID NO: 18.

Exemplary humanized antigen-binding fragments include fragments with a heavy chain variable region having an amino acid sequence as in SEQ ID NO: 17 and a light chain region having an amino acid sequence as in SEQ ID NO: 21.

In some embodiments, the humanized antigen-binding fragments include a linker region. In some embodiments, the linker region links the heavy chain variable region with the light chain variable region. In some embodiments, the linker region links the heavy chain with the light chain. In some embodiments, the linker region comprises SEQ ID NO: 25. In some embodiments, the linker region comprises SEQ ID NO: 26. In some embodiments, the linker region links the heavy chain variable region with the light chain variable region, and the linker comprises SEQ ID NO: 25. In some embodiments, the linker region links the heavy chain with the light chain, and the linker comprises SEQ ID NO: 26.

The antibodies or antigen-binding fragments thereof described herein can have binding affinities (in M) for PCLA that include a dissociation constant (KD) of less than 1×10⁻⁵. In some embodiments, the KD is less than 1×10⁻⁶, 2×10⁻⁶, 3×10⁻⁶, 4×10⁻⁶, 5×10⁻⁶, 6×10⁻⁶, 7×10⁻⁶, 8×10⁻⁶, or 9×10⁻⁶. In some embodiments, the KD is less than 1×10⁻⁷, 2×10⁻⁷, 3×10⁻⁷, 2×10⁻⁷, 3×10⁻⁷, 4×10⁻⁷, 5×10⁻⁷, 6×10⁻⁷, 7×10⁻⁷, 8×10⁻⁷, or 9×10⁻⁷. In some embodiments, the KD is less than 1×10⁻⁸, 2×10⁻⁸, 3×10⁻⁸, 4×10⁻⁸, 5×10⁻⁸, 6×10⁸, 7×10⁻⁸, 8×10⁻⁸, or 9×10⁻⁸. In some embodiments, the KD is less than 1×10⁻⁹, 2×10⁻⁹, 3×10⁻⁹, 4×10⁻⁹, 5×10⁻⁹, 6×10⁻⁹, 7×10⁻⁹, 8×10⁻⁹, or 9×10⁻⁹. In some embodiments, the KD is less than 1×10⁻¹⁰, 2×10⁻¹⁰, 3×10⁻¹⁰, 2×10⁻¹⁰, 3×10⁻¹⁰, 4×10⁻¹⁰, 5×10⁻¹⁰, 6×10⁻¹⁰, 7×10⁻¹⁰, 8×10⁻¹⁰, or 9×10⁻¹. In some embodiments, the KD is less than 1×10⁻¹¹, 2×10⁻¹¹, 3×10⁻¹¹, 4×10⁻¹¹, 5×10⁻¹¹, 6×10⁻¹¹, 7×10⁻¹¹, 8×10⁻¹¹, 9×10⁻¹¹, 1×10⁻¹², 1×10⁻¹³, 1×10⁻¹⁴, or 1×10⁻¹⁵.

As described in the Examples, the humanized F77 antibody binds to PC-3 cells as measured by flow cytometry.

### Chimeric Antigen Receptors

Disclosed herein are chimeric antigen receptors (CARs) comprising the disclosed scFvs. The CARs can comprise any of the discloses scFvs and a transmembrane domain. The CARs can comprise any of the discloses scFvs, a transmembrane domain, and a signal transduction domain. The CARs can comprise any of the discloses scFvs, a transmembrane domain, and an immune-costimulatory domain.

For example, the CAR can comprise: a) the humanized antigen-binding fragments comprising the heavy chain CDR1, CDR2, and CDR3 of SEQ ID NOs: 8, 9, and 11, and the light chain CDR1, CDR2, and CDR3 of SEQ ID NOs: 12, 14, and 15, and b) a co-stimulatory domain comprising the amino acid sequence of SEQ ID NO: 42 or 43.

The CAR can comprise: a) the humanized antigen-binding fragments comprising the heavy chain CDR1, CDR2, and CDR3 of SEQ ID NOs: 7, 9, and 11, and the light chain CDR1, CDR2, and CDR3 of SEQ ID NOs: 12, 14, and 15, and b) a co-stimulatory domain comprising the amino acid sequence of SEQ ID NO: 42 or 43.

The CAR can comprise: a) the humanized antigen-binding fragments comprising the heavy chain CDR1, CDR2, and CDR3 of SEQ ID NOs: 7, 10, and 11, and the light chain CDR1, CDR2, and CDR3 of SEQ ID NOs: 13, 14, and 16, and b) a co-stimulatory domain comprising the amino acid sequence of SEQ ID NO: 42 or 43.

In some embodiments, the CAR comprises an scFv comprising the heavy chain variable region comprising SEQ ID NO: 1, the light chain variable region comprising SEQ ID NO: 2, and a co-stimulatory domain comprising the amino acid sequence of SEQ ID NO: 42 or 43.

In some embodiments, the CAR comprises an scFv comprising the heavy chain variable region comprising SEQ ID NO: 3, the light chain variable region comprising SEQ ID NO: 4, and a co-stimulatory domain comprising the amino acid sequence of SEQ ID NO: 42 or 43.

In some embodiments, the CAR comprises an scFv comprising the heavy chain variable region comprising SEQ ID NO: 5, the light chain variable region comprising SEQ ID NO: 6, and a co-stimulatory domain comprising the amino acid sequence of SEQ ID NO: 42 or 43.

The disclosed CARs can comprise the heavy chain variable region comprising SEQ ID NO: 17, the light chain variable region comprises SEQ ID NO: 18, and a co-stimulatory domain comprising the amino acid sequence of SEQ ID NO: 42 or 43.

The disclosed CARs can comprise a heavy chain framework region that has about 95% sequence identity to the framework regions of SEQ ID NO: 23 or SEQ ID NO: 24, and a co-stimulatory domain comprising the amino acid sequence of SEQ ID NO: 42 or 43.

The disclosed CARs can comprise a linker region comprising SEQ ID NO: 25 or SEQ ID NO: 26.

In some embodiments, the CAR comprises an amino acid sequence of SEQ ID NO: 38, 39, 40 or 41. In some embodiments, the CAR is encoded by a nucleic acid molecule comprising SEQ ID NO: 34, 35, 36, or 37.

### Compositions Comprising the Disclosed Antibodies or Antigen-Binding Fragments

Disclosed herein are compositions comprising any of the disclosed antibodies or antigen-binding fragments thereof. The disclosed compositions can be used, for example, for diagnosis or treatment of prostate cancer. The compositions can comprise any of the disclosed clones of the murine F77 monoclonal antibody, the humanized antibodies derived from the murine F77 monoclonal antibody, or antigen-binding fragments thereof, and a pharmaceutically acceptable excipient.

The compositions can comprise any of the disclosed scFvs. The compositions can comprise any of the disclosed scFabs.

The compositions may include the clones of the murine F77 antibody, the humanized antibodies derived from the murine F77 antibody, or the antigen-binding fragments thereof linked to a therapeutic and/or diagnostic agents. Therapeutic or diagnostic agents include agents for radiotherapy, surgery, cytostatic agents and cytotoxic agents. The agent may be linked to the murine F77 antibody, the humanized antibodies derived from the murine F77 antibody, or the antigen-binding fragments thereof via a covalent or a non-covalent bond. The agent may be useful for radiological visualization methods, such as SPECT/PET, computed tomography (CT), ultrasound (US), and/or magnetic resonance imaging (MRI).

The compositions also include one or more pharmaceutically or physiologically acceptable excipients or carriers. The disclosed compositions can be formulated so as to be suitable for use in a variety of delivery systems. Suitable formulations for use are found in Remington's Pharmaceutical Sciences, Mack Publishing Company, Philadelphia, Pa., 17th ed. (1985). Exemplary excipients include buffered aqueous solutions, saline solutions, carriers, and antioxidants. Exemplary antioxidants include alpha tocopherol, ascorbic acid, ascorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and sodium sulfite.

The pharmaceutical compositions may be administered by various routes, e.g., by injection for systemic or local delivery. The preferred routes of administering the pharmaceutical compositions include subcutaneous, intramuscular, intravenous, intraperitoneal, and intratumoral injection.

### Methods of Making Humanized Antibodies

Methods for humanizing non-human antibodies are known in the art. For example, humanized antibodies can be generated by substituting rodent complementarity-determining regions (CDRs) in human antibodies so that the CDR sequences are grafted in the corresponding regions in a human antibody. Detailed procedures are disclosed in Jones et al., Nature, 321, 522-525 (1986); Riechmann et al., Nature, 332, 323-327 (1988); Verhoeyen et al., Science, 239, 1534-1536 (1988).

Methods that can be used to produce humanized antibodies are also described in U.S. Pat. Nos. 4,816,567; 5,565,332; 5,721,367; 5,837,243; 6,130,364; and 6,180,377.

The Examples describe one such method of humanizing the murine F77 monoclonal antibody, clone 129.16.

Human, chimeric, or humanized derivatives of murine F77 monoclonal antibody are particularly preferred for in vivo use in humans, however, murine antibodies may be advantageously employed for many uses (for example, in vitro or in situ detection assays, acute in vivo use, etc.). A humanized antibody can comprise amino acid residue substitutions, deletions or additions in one or more non-human CDRs. The humanized antibody derivative may have substantially the same binding, stronger binding or weaker binding when compared to a non-derivative humanized antibody. In specific embodiments, one, two, three, four, or five amino acid residues of the CDRs, or of the variable fragment regions, have been substituted, deleted or added (i.e., mutated).

Methods of Using the Antibodies and their Fragments

Disclosed herein are methods of using any of the disclosed clones of the murine F77 monoclonal antibody, the humanized F77 antibodies, and the antigen-binding fragments thereof. In some embodiments, the methods comprise diagnosing a subject with prostate cancer by administering to the subject an effective amount of any of the disclosed clones of the murine F77 monoclonal antibody, humanized F77 antibodies, or an antigen-binding fragments thereof. In some embodiments, the methods comprise treating a subject with prostate cancer by administering to the subject an effective amount of any of the disclosed clones of the murine F77 monoclonal antibody, humanized F77 antibodies, or an antigen-binding fragments thereof.

In some embodiments, the methods include administering an effective amount of any of the disclosed scFv fragments, any of the disclosed scFab fragments, any of the disclosed humanized scFv fragments, or any of the disclosed humanized scFab fragments to a subject.

In The term “subject” as used herein is intended to mean any animal, in particular, mammals. The antibodies and antigen-binding fragments thereof, and the methods described herein, are applicable to human and nonhuman animals, although preferably used with mice and humans, and most preferably with humans. “Subject” and “patient” are used interchangeably herein. In some embodiments, the subject is human.

In some embodiments, the subject has or suffers from prostate cancer. In some embodiments, the subject has localized (stages I and II), locally advanced (stage III), or advanced (stage IV) prostate cancer.

It is to be understood that the embodiments described herein are not limited to particular methods, reagents, compounds, compositions or biological systems, which can, of course, vary. It is also to be understood that the terminology used herein is for the purpose of describing the antibodies and antigen-binding fragments thereof, and methods of detecting and/or diagnosing and/or treating, and is not intended to be limiting.

### EXAMPLES

### Example 1. Purification and Crystallization of F77Fab and its Complex with GSC-915

### Materials and Methods

Mouse monoclonal antibody F77 was prepared from hybridoma and purified using HiTrap Protein A HP. The antibody was dialyzed against a buffer containing 20 mM sodium phosphate pH 6.5 and 150 mM sodium chloride overnight, and was digested with papain in 10 mM cysteine-HCl at room temperature for 3.5 hours. The Fab fragment was separated from the digestion solution using Superdex 200 10/300 GL and HiTrap Protein A HP. Subsequently, F77Fab was desalted, concentrated to 12 mg/ml in 5 mM Tris-HCl, pH 7.5 buffer, and crystallized by hanging drop vapor diffusion at 16° C. In brief, the antibody was mixed 1:1 with mother liquor (15% polyethylene glycol 4000, 0.15 M ammonium sulfate, 0.1 M Tris-HCl, pH 8.0) and equilibrated against the reservoir solution same as mother liquor. Plate shaped crystals appeared after several days. A dataset was collected from a single crystal of the antibody at 100 K on beamline F1 at the Cornell High Energy Synchrotron Source (MacCHESS). The diffraction data were processed using HKL2000.

The crystal structure of F77Fab was determined by molecular replacement using Molrep with the coordinates of the Fab fragment of anti-Digoxin antibody 40-50 (PDB code: 1ibg) as a search model. Molecular replacement yielded two solutions, corresponding to the expected two Fab fragments in one asymmetric unit. The model was then subjected to structure refinement using the programs Refmac with 5% of the reflection data randomly chosen for Rfree calculation. Model building and real space refinement were carried out with Coot referring to the sigma weight 2Fo-Fc maps. The parameter of data collection and structure refinement statistics is shown in Table. 1.

TABLE 1
Data collection and Structure refinement statistics

| Parameters | Values |
|---|---|
| Data collection | |
| Space group | P21 |
| Cell dimensions | |
| a, b, c (Å) | 91.88, 47.63, 129.80 |
| α, β, γ (°) | 90, 102.4, 90 |
| Resolution (Å) | 50-2.35(2.39-2.35)^{a} |
| R_{merge} | 0.078(0.699) |
| Average I/σI | 13.8(1.7) |
| Completeness (%) | 99.8(99.6) |
| Structure Refinement | |
| Resolution(Å) | 20-2.35 |
| Unique reflections | 44757 |
| Unique reflections for R_{free} | 2167 |
| No. of protein atoms | 6192 |
| R_{crystallography} (%) | 19.0 |
| R_{free} (%) | 24.7 |
| R.M.S deviations | |
| Bond length (Å)/angle (°) | 0.010/1.355 |
| Average B factor (Å²) | 38.70 |
| Ramachandran plot | |
| Most favored (%) | 97.47 |
| Allowed (%) | 0.58 |

### Results

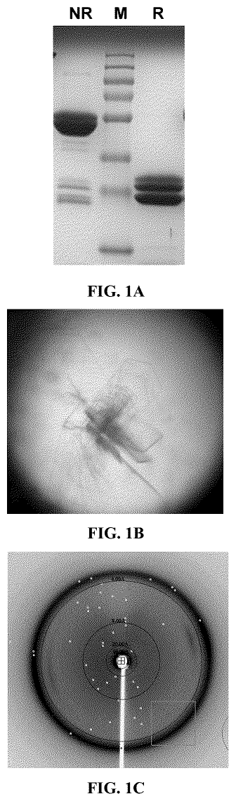

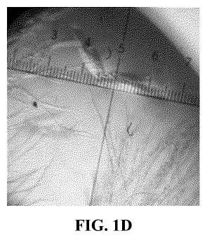

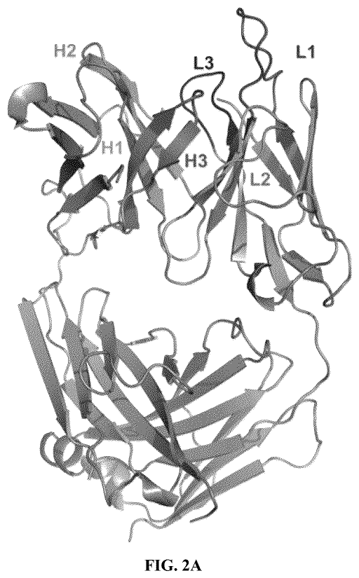

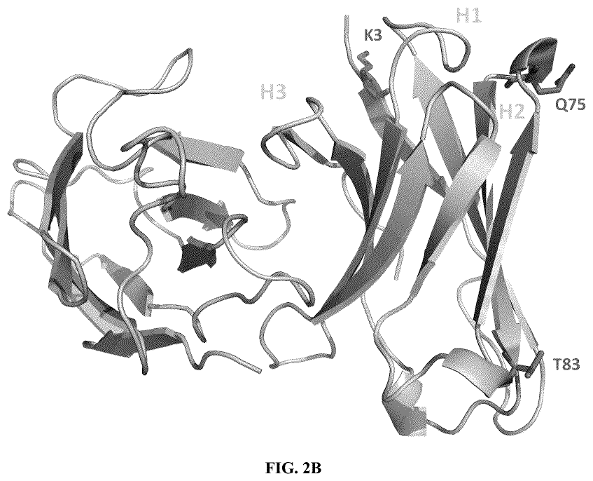

The findings from the purification and crystallization of F77Fab and its complex with GSC-915 are shown in FIG. 1A-1D. The crystal structure of mouse F77 Fab is shown in FIG. 2A.

### Example 2. Humanization of F77

### Materials and Methods

A human germline acceptor sequence similar to the heavy and light chain V gene region or J gene region sequences of the mouse F77 amino acid sequence was searched by IMGT. Then, humanized monoclonal antibody sequence was generated by grafting mouse antibody heavy chain CDR1, CDR2, CDR3 and antibody light chain CDR1, CDR2, CDR3 to human germline framework followed by key mutations in framework. The heavy chain and light chain of mouse F77 Fv regions are aligned to their corresponding human germline sequences (IGHV4-4*8, IGKV2-30*02) (shown below).

A humanized antibody named F77N76S and wild type F77 (murine HC, human LC)

(Human LC sequence:
SEQ ID NO: 21
DVVMTQSPSSLSVTLGQPASISCRSSQTLVHSNGNTFLHWFQQRPG
QSPRRLIYKVSNRFSGVPDRFSGSGSGTDFTLTISRVEAEDVGVY
YCSQGTHAPFTFGGGTKVEIKRRVATPSVFIFPPSDEQLKSGTAS
VVCLLNNFYPREAKVQWKVDNALQSGNSQESVTEQDSKDSTYSLS
STLTLSKADYEKHKVYACEVTHQGLSSPVTKSFNRGEC,)

were expressed in 293T cells and purified using HiTrap Protein G HP.

Based on the comparison of murine F77 heavy chain variable sequence and human antibody sequences (consensus H3), several further amino acid residues were identified in the murine F77 sequence to be mutated to obtain a humanized F77 antibody (sequence alignment shown below). The three most different residues reside on the surface of F77 Fv. These sites are potential immunogenic sites and mutations at these sites are unlikely to affect the F77 binding as they are away from the CDR regions (FIG. 2B). Two mutant heavy chain constructs, a double mutant Q75K/T83N and triple mutant K3Q/Q75K/T83N were obtained. These mutants were expressed as a fusion protein containing the human IgG1 Fc region and in a complex with a humanized F77 light chain (SEQ ID NO: 21) in 293T cells. The wild type sequence was also expressed as a control (murine heavy variable, human IgG1 Fc, human LC SEQ ID NO: 21). These expressed IgG molecules were purified using HiTrap Protein G HP. The binding activity of these proteins to PC3 cells was tested by FACS, which showed that the humanized antibody binds to PC-3.

### Results

Fv region sequence alignment of mouse F77, clone 129.16, heavy chain (mF77H, SEQ ID NO: 29) and light chain (mF77k, SEQ ID NO: 30) with their corresponding human germline sequences (hIGHV4-4*08, SEQ ID NO: 31, and hIGKV2-30*02, SEQ ID NO: 32) is shown below. The CDR regions are underlined.

| | | |
|---|---|---|
| mF77H | EVKLVESGPGLVAPSQSLSITCTVSGFSLTYYGVHWGRQSPGKGLEWLGIIWAGGNTNYN | 60 |
| hIGHV4-4*08 | QVQLQESGPGLVKPSETLSLTCTVSGGSISSYYWSWIRQPPGKGLEWIGYIYTSGSTNYN | 60 |
| | :*:* ******* **::**:****** *:: * * * ** *******:* *::.*.**** | |
| mF77H | STLKSRLSISKDNSQSQVFLKMTSLQTDDTAMYYCARDDYAAMDYWGQGT----- | 110 |
| hIGHV4-4*08 | PSLKSRVTISVDTSKNQFSLKLSSVTAADTAVYYCAR---YFDYWGQGSLVTVSS | 112 |
| | :****::** *.*:.*. **::*: : ***:***** :******: | |
| mF77k | ELVMTQSPLSLPVSLGDQASISCRSSQTLVHSNGNTFLHWYLKKPGQSPKLLIYKVSNRF | 60 |
| hIGKV2-30*02 | DVVMTQSPLSLPVTLGQPASISCRSSQSLVHSDGNTYLNWFQQRPGQSPRRLIYKVSNRD | 60 |
| | ::***********:**: *********:****:***:*:*: ::*****: ******** | |
| mF77k | SGVPDRFSGSGSGTHFTLKISRVEAEDLGVYFCSQGTHAPFTFGGGTKLEIK | 112 |
| hIGKV2-30*02 | SGVPDRFSGSGSGTDFTLKISRVEAEDVGVYYCMQGTHWPCTFGQGTKLEIK | 112 |
| | **************.************:***:* **** * *** ******* | |

The crystal structure of mouse F77 Fab is shown in FIG. 2B. Sequence alignment of murine F77 Fv (mF77H, SEQ ID NO: 3) and a consensus human variable region sequence (consensus H3, SEQ ID NO: 24) is shown below. The CDR regions are underlined, the three identified amino acid residues in mF77H are indicated with the misalignment asterisks. A sequence with all three residues mutated is also listed (mF77H mutant, SEQ ID NO: 33):

| Consensus H3 | EVQLVESGGGLVQPGGSLRLSCAASGF. . . . WVRQAPGKGLEWVGAI. . . . . | |
|---|---|---|
| mF77H | EVKLVESGPGLVAPSQSLSITCTVSGFSLTYYGVHWGRQSPGKGLEWLGIIWAGGNTNYN | 60 |
| mF77Hmutant | EVQLVESGPGLVAPSQSLSITCTVSGFSLTYYGVHWGRQSPGKGLEWLGIIWAGGNTNYN | 60 |
| | **:********************************************************* | |
| Consensus H3 | . KGRFTISRDNSKNTLYLQMNSLRAEDTAVYYCAR. . . FDYWGQGTL | |
| mF77H | STLKSRLSISKDNSQSQVFLKMTSLQTDDTAMYYCARDDYAAMDYWGQGTS | 111 |
| mF77Hmutant | STLKSRLSISKDNSKSQVFLKMNSLQTDDTAMYYCARDDYAAMDYWGQGTS | 111 |

### Example 3. Binding of Humanized F77 Antibody to PC-3 Cells

### Materials and Methods

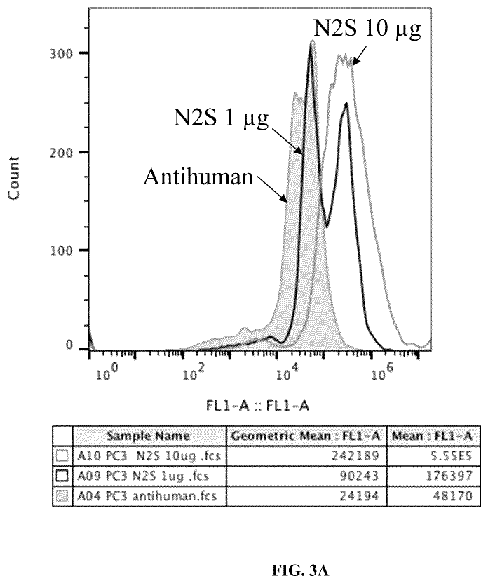

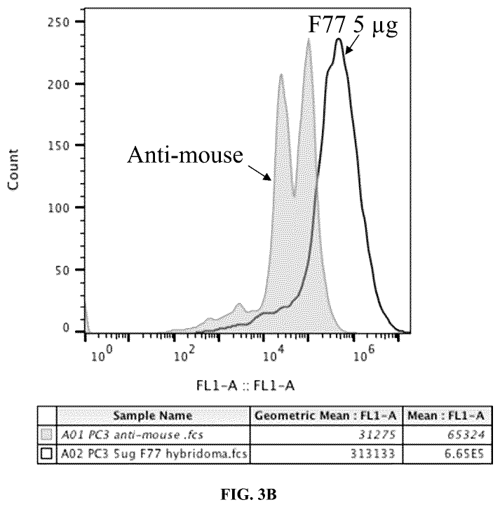

Binding assay of F77 to PC-3 cells. After PC-3 cells' Fc receptors were blocked for 5 minutes with 5% FBS/PBS, PC-3 cells were incubated for 30 minutes with either (FIG. 3A) humanized F77N76S (labeled as N2S), or (FIG. 3B) F77 from hybridoma. The primary antibodies were diluted in 5% FBS/PBS to the indicated concentrations. Cells were washed twice with 1% BSA/PBS. Afterwards, cells were incubated for 30 minutes with anti-human IgG, Fcγ fragment conjugated to fluorescein (Jackson ImmunoResearch) (F77 in FIG. 3B is the mouse antibody, and anti-mouse IgG secondary antibody was used for FIG. 3B). Cells were then washed twice with 1% BSA/PBS, resuspended in FACS buffer, and analyzed using a BD LSR at 530 nm±15 nm.

### Results

The binding activity of F77N76S and the wild type F77 to PC-3 cells was assayed using FACS (FIG. 3A and FIG. 3B). The relationship curves between the antibody concentration and the geometric mean obtained in FACS experiments showed that F77N76S had similar binding activity to PC-3 as the wild type F77. The relationship curves between antibody concentration and geometric mean obtained in FACS experiments showed that F77N76S have kept binding activity to PC-3 of F77.

### Example 4. Production of Single Chain Fv and Single Chain Fab of F77

### Materials and Methods

The scFv construct of F77 was generated and contained the variable region of heavy chain, a linker of 20 residues and the variable region of light chain from N-terminus to C-terminus. The scFab construct was generated and contained the variable region and constant 1 region of the heavy chain, a linker of 30 residues and the whole light chain.

### Results

The constructs for the scFv and scFab of F77 are presented below in Tables 2 and 3. The heavy chain, linker and light chain sequences are presented with the linker sequences underlined.

### Example 5. Development of CAR T-Cell Therapy for Prostate Cancer

F77, a carbohydrate antigen, is specifically expressed on both androgen-dependent and androgen-independent prostate cancer cells, making it a target for immunotherapy of prostate cancer. The current example entails the generation and evaluation of a second-generation CAR-directed against a carbohydrate antigen expressed on the surface of malignant prostate cancer cells. Using a single chain variable fragment (scFv) derived from an F77-specific mouse monoclonal antibody, a second generation CARs with both CD28 and CD137 (4-1BB) costimulatory signals was generated. Anti-F77 CAR T cells generated by lentiviral CAR delivery to primary human T cells produced cytokines and killed tumor cells in an F77 expression-dependent manner. Moreover, F77-specific CAR T cells eradicated tumors in a human xenograft model of prostate cancer using the PC3 cells. These results show that the F77 carbohydrate antigen as an immunotherapeutic target for the treatment of prostate cancer and other tumors that express this aberrant carbohydrate structure.

This example focuses on the development of chimeric antigen receptor (CAR) T cells to target prostate tumors expressing the F77 antigen. For establishing this, F77-55.10 scFv was used.

Monoclonal antibodies against carbohydrate structures on malignant cells have previously been reported to target tumor cells through antibody-dependent cellular cytotoxicity (ADCC) and antibody-dependent phagocytosis by macrophages. One such cytotoxic antibody is the murine mAb F77 129, IgG3 subtype kappa, raised against the PC3 human adenocarcinoma recognizing a unique carbohydrate antigen, highly restricted to both androgen-independent (PC3, PC3-MM2, DU145) and androgen-dependent (LnCaP) prostate cancer cell surfaces. Immunohistological studies with mAb F77 129 showed staining of >90% of primary prostate cancers, with only limited focal staining of benign prostate tissues, and 29 of 34 metastatic human prostate cancer specimens also stained positive. Normal or tumor tissues of human colon, kidney, cervix, pancreas, lung, skin, and bladder were not stained with mAb F77 129 confirming that the antigen recognized by mAb F77 is highly restricted to prostate and is overexpressed in prostate tumors. This antibody can significantly inhibit androgen-independent tumor growth in both PC3 and Du145 mouse tumor xenograft models, with no activity against an F77-negative xenograft and employs a CD44-dependent mechanism to induce tumor cell apoptosis. A sandwich ELISA-based assay employing mAb F77 129 identified a glycosylated form of CD44 from both prostate cancer cells and sera from men with prostate cancer substantiating the potential therapeutic utility mAb F77 129 for detection of prostate cancer.

mAb F77 55.10 is a murine mAb F77, IgG3 subtype kappa. Like mAb F77 129, mAb F77 55.10 shows high binding to PC3 prostate carcinoma.

In this example, the mAb F77 55.10 scFv was used to create second-generation CAR targeting F77 carbohydrate antigen, which besides the CD3 (chain contains the signaling domains of 4-1BB or CD28. This example shows that primary T lymphocytes isolated from the peripheral blood of healthy volunteers expressing F77 scFv CAR induce in vitro and in vivo tumor reactivity against androgen independent prostate cancer cells.

### Materials and Methods

Cloning of F77 55.10 scFv in Bicistrionic Lentiviral Vector

Sequencing of mAb F77 55.10 was performed by Yurogen Biosytems LLC (Worcester, USA). The heavy chain and light chain variable-region sequences of the antibody were used to design single chain variable fragments (scFv) with the following pattern: heavy chain variable region-linker-light chain variable region (VHVL) and light chain variable region-linker-heavy chain variable region (VLVH). The linker had the following amino acid sequence: GGGGSGGGGSSGGGS (SEQ ID NO: 44). DNA encoding F77 scFvs was codon optimized and synthesized by GENEWIZ with BamHI and NheI restriction sites. These DNA sequences were cloned into a bicistronic, 3^{rd} generation lentiviral vector to generate a vector that expresses a CAR with either a 4-1BB or CD28 immuno-costimulatory domain as previously described and cyan fluorescent protein (CFP) separated by a P2A ribosomal skip sequence. The sequence of each of the CAR-encoding lentiviral vector insert is shown schematically in FIG. 15.

Both F77 55.10 scFv's—VHVL and VLVH were cloned in a bicistronic lentiviral vector using a two-step cloning strategy. Both scFv's were cloned with 4-1BBzeta and CD28zeta immuno-costimulatory domains (to generate constructs having nucleic acid sequences of SEQ ID NOs: 34, 35, 36, and 37) to compare which orientation of scFv and which immuno-costimulatory domain functions better in downstream assays.

Step 1 involved the construction of an intermediate plasmid. For this, pELPS-F77 VHVL CD28zeta and pELPS-F77 VLVH CD28zeta plasmid DNA were digested with BamHI HF and NheI HF restriction enzymes at 37° C. for 3 hrs to obtain BamHI-F77 VHVL-NheI and BamHI-F77 VLVH-NheI insert fragments (˜700 bp), respectively. Similarly, pTRPE-806-41BBzeta and pELPS-2176-CD28zeta plasmid DNA (obtained from Selene Nunez Cruz, Milone lab) were digested with BamHI HF and NheI HF restriction enzymes to obtain, pTRPE-BamHI-NheI-41BBzeta and pELPS-BamHI-NheI-CD28zeta (˜8000 bp) vector backbones, respectively. The digested DNA were purified using QIAquick Gel extraction kit followed by estimation on 1% TAE-agarose gel. The ligation was set up in a reaction volume of 10⁻²⁰ μl using T4 DNA Ligase (5U/μl) for 1 hr. The ligation mixture was then transformed in E. coli Stbl3 CaCl₂) competent cells. The resultant recombinants were named as pTRPE F77VHVL 41BBzeta, pTRPE F77VLVH 41BBzeta, pELPS F77VHVL CD28zeta and pELPS F77VLVH CD28zeta, respectively. These recombinants were characterized by sequencing of plasmid DNA with EF1alpha promoter primer, 442-reverse primer, F77 VL start primer and F77 VH end primer. These intermediate plasmids were then transformed in E. coli Top10 CaCl₂) competent cells. Plasmid DNA for each of these intermediate vectors was prepared using QIAprep Spin MiniPrep kit.

Step 2 involved cloning of F77 scFv's from the intermediate plasmids into CFP bicistronic lentiviral vectors. For this, pTRPE F77VHVL 41BBzeta, pTRPE F77VLVH 41BBzeta, pELPS F77VHVL CD28zeta and pELPS F77VLVH CD28zeta plasmids were digested with BamHI and SalI restriction enzymes at 37° C. for 16 hrs to obtain BamHI-F77VHVL 41BBzeta-SalI, BamHI-F77VLVH 41BBzeta-SalI, BamHI-F77VHVL CD28zeta-SalI, and BamHI-F77VLVH CD28zeta-SalI, insert fragments (˜1500 bp), respectively. Similarly, pTRPE-CFP-P2A-3C10⁻⁴¹BBzeta plasmid DNA (obtained from Selene Nunez Cruz, Milone lab) was digested with BamHI and SalI restriction enzymes to obtain, pTRPE-CFP-P2A-BamHI-SalI (˜9000 bp) vector backbone. The digested DNA were purified using QIAquick Gel extraction kit followed by estimation on 1% TAE-agarose gel. The ligation was set up in a reaction volume of 15-20 μl using T4 DNA Ligase (5U/μl) for 1 hr. The ligation mixture was then transformed in E. coli Top10 CaCl₂) competent cells. The resultant recombinants were named as pTRPE-CFP-P2A-F77VHVL 41BBzeta, pTRPE-CFP-P2A-F77VLVH 41BBzeta, pTRPE-CFP-P2A-F77VHVL CD28zeta and pTRPE-CFP-P2A-F77VLVH CD28zeta, respectively. These recombinants were characterized by sequencing of plasmid DNA with EF1alpha promoter primer, 442-reverse primer, F77 VL start primer, F77 VH end primer, P2A start primer and CD8 Hinge primer. The DNA for each of these final plasmids was prepared using QIAGEN HiSpeed Plasmid Maxi kit.

### Lentiviral Supernatant Production

These biscistronic plasmids were then used for lentiviral supernatant production by transfection in HEK293T cells. To produce supernatant containing replication defective lentiviruses, a third-generation lentiviral transfer plasmid encoding appropriate CAR was transfected into HEK293T cells (ATCC) using Lipofectamine 2000 (Invitrogen) with three packaging plasmids: pRSV-rev (encoding rev), pMDLgal/pRRE (encoding gag and pol), pMDG.1 (encoding the vesicular stomatitis virus envelope VSV-G). The supernatant was harvested 24 and 48 hrs post-transfection, filtered using 0.45 μM syringe filter, centrifuged at 12,000 rpm for 24 hours, pelleted, resuspended in collecting medium and stored at −80° C.

### T Cell Transductions and Expansion

Bulk T cells were isolated by negative selection using RosetteSep T cell enrichment cocktail (StemCell Technologies, Vancouver, BC, Canada). T cells were activated using anti-CD3/anti-CD28 coated Dynabeads (Thermo Fisher Scientific, Waltham, MA, USA) at a bead:cell ratio of 3:1 and cultured in RPMI1640 with 10% fetal bovine serum (FBS), 10 mM HEPES, and 1% penicillin/streptomycin. 24 hrs after activation, the T cells were transduced with lentiviruses at a MOI of 2 to 3. T cells were then expanded in media containing 100 IU/mL IL-2 (Proleukin) until resting for 10⁻¹⁴ days, with a cellular volume of approximately 400 fL, prior to cryopreservation. Rested and frozen T cells were thawed and washed with media prior to functional in vitro and in vivo assays.

Five lentivirus preparations were used for this assay: pTRPE-CFP-P2A-F77VHVL 41BBzeta, pTRPE-CFP-P2A-F77VLVH 41BBzeta, pTRPE-CFP-P2A-F77VHVL CD28zeta, pTRPE-CFP-P2A-F77VLVH CD28zeta and 2173-CD28zeta (EFGR-VIII; serves as negative control for the assay). For each transduction, 400 μl of lentiviral particles were added to 3×10⁶ activated T cells and incubated at 37° C., 5% CO₂ for 48 hrs. For 9 days post transduction, cells were stimulated with anti-CD3/anti-CD28 magnetic beads in presence of 100 IU/ml of IL2. The CART cells were then debeaded and frozen for downstream assays like flow cytometry to analyze F77 scFv surface expression, release of hIFNgamma by ELISA assay and cytotoxicity assay.

### Staining and Flow Cytometric Analysis

PBS-washed pelleted T cells on day 8 of expansion were stained with biotin-SP-AffiniPure Goat Anti-Mouse IgG F(ab′)₂ Fragment Specific (Jackson ImmunoResearch Laboratories, Inc.) primary antibody for 60 minutes at room temperature. Cells were then washed three times in 2 mL room temperature PBS and labeled with R-phycoerythrin-conjugated streptavidin (SA: PE; BD Biosciences) for 60 minutes at room temperature. All F77 CAR constructs have a cyan fluorescent protein (CFP) tag and fluorescence was read in the CFP channel. 2173-CD28zeta (EGFRvIII) CAR T cells were stained with Alexa Fluor 488 AffiniPure Goat Anti Human IgG F(ab′₂) (Jackson ImmunoResearch Laboratories, Inc.). Flow cytometry analysis was performed using LSRFortessa C (BD Biosciences) and analyzed with FlowJo X 10.5.3 (Tree Star, Ashland OR).

Surface expression of F77 antigen was compared among different prostate cancer cell lines; PC3, RWPE-1, RWPE-2 and PC3 FUT1 KO cell lines by staining with purified mAb F77 55.10 using the same method as described earlier. Briefly, cells were trypsinized, washed twice with PBS, and then fixed with 4% paraformaldehyde in PBS at room temperature for 15 min. Following washing with PBS, cells were blocked with 10% goat serum for 30 min. Approximately 0.5-1×10⁶ cells were resuspended in 100 μL FACS buffer (1% BSA/PBS solution) and treated with 5 μg of the purified mAb F77 55.10. Cells were thoroughly mixed and incubated at 4° C. for an hour. Thereafter, the cells were washed twice with FACS buffer, and subsequently they were incubated for an hour at 4° C. with FITC-labeled goat anti-mouse antibody (1:100; Jackson ImmunoResearch Laboratories, Inc.). Using FACS buffer, the cells were washed twice, and flow cytometry analysis was performed using BD Accuri (BD Biosciences) and analyzed with FlowJo X 10.5.3 (Tree Star, Ashland OR).

### Interferon (IFN) γ Release ELISA

The CAR T cells were co-cultured with target cells at an E: T ratio of 10:3 in flat bottom 96-well plates for 16 hrs at 37° C. and 5% CO₂. The supernatants were collected, diluted 1:5 and subjected to IFN γ detection by sandwich ELISA assay using Human IFN-gamma DuoSet ELISA kit (R&D) according to manufacturer's instructions. Briefly, a 96-well ELISA plate was coated with 2 μg/ml of mouse anti-human IFN γ capture antibody. Supernatant from co-cultures of effector cell and target cell was added over the capture antibody coated wells and incubated at RT for 2 hrs. The wells were washed followed by addition of biotinylated mouse anti-human IFN γ detection antibody for 1 hr at RT. The bound sample was detected by streptavidin-HRP followed by addition of colored substrate. The reaction was terminated using a stop solution and optical density was measured at 450 nm in a multimode reader. Each individual experiment was performed in duplicates.

Impedance Based Real Time Potency Assessment Using xCELLigence Real-Time Cell Analysis (RTCA)

The xCELLigence RTCA instrument (Agilent Technologies) was utilized for all impedance experiments. The RTCA system utilizes cellular impedance readout to monitor real-time changes in target cell number, cell size, and cell-substrate attachment strength using a parameter called Cell Index (CI) to reflect the viability of target cancer cells. First, 50 μL of target cancer cell culturing media was added to each well of E-Plate VIEW 96 plate (Agilent Technologies) and the background impedance was measured. Adherent target cells were seeded in the E-Plate VIEW 96 plate at a density of 10,000 cells/well (PC3 GFP, PC3 FUT1 KO GFP, RWPE-1 GFP, RWPE-2 GFP and U87EGFRvIII GFP [cells expressing EGFRvIII; mutant EGFR]) in a volume of 50 μL and incubated at 37° C., 5% CO₂. After monitoring cell proliferation for 24 hrs, CAR T (effector) cells T cells (NTD [Non transduced T cells; T cells without any CAR], F77 VLVH 4-1BBzeta, F77 VHVL CD28zeta and 2173 CD28zeta (EGFRvIII CAR; negative CAR control for the prostate cancer cell lines) were added at several E:T ratios of 15:1, 10:1 and 5:1 in duplicates. E:T ratio corresponds to effector cell (CAR T cell) to target cancer cell ratio. A full lysis control was included by adding 1% tween-20 to target cells. Impedance was measured every 15 min, and images were acquired every 2 hrs. Exposure times were as follows: green (300 ms), brightfield (automatically optimized by the eSight software). Changes in impedance were reported as Cell Index (CI) and Normalized Cell Index (NCI). Impedance-based measurements of the normalized cell index were collected every 15 min, which were determined by measuring of the impedance of current across the plate caused by target cell adherence.

Impedance was calculated by % Cytolysis.

$$\% Cytolysis = [ 1 - Normalized CI treatment / Normalized CI target only ] \times 100.$$

$$( CI = Cell Index )$$

### In Vivo Tumor Model

NSG (NOD-SCID-Il2rg−/−) mice were used. Six- to eight-week-old NSG mice were injected subcutaneously with 5×10⁶ PC3 cells in 100 μL PBS. On day 14 after tumor implantation when tumors reached a volume of 200 mm³, mice were randomly assigned and intratumorally injected with 5×10⁶ CAR T cells: non transduced (NTD; T cells without any CAR) or F77 VLVH 4-1BBzeta or F77 VHVL CD28zeta (40-60% transduction efficiency). Tumor volume was measured by caliper using the formula (π/6)×(length)×(width)² at the indicated times (n=5 mice per group).

### Statistical Analysis

All statistical analyses were performed using Prism v9 (GraphPad Software). Statistical comparisons between two groups were determined by two-tailed student's t-test. Data generated from the xCELLigence instrument are reported as the average and SEM of the experiments.

### Results

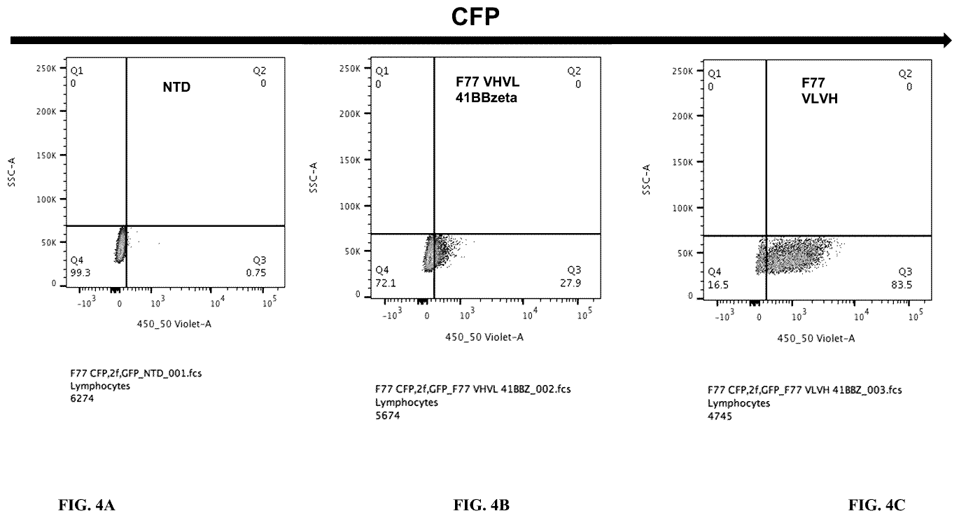

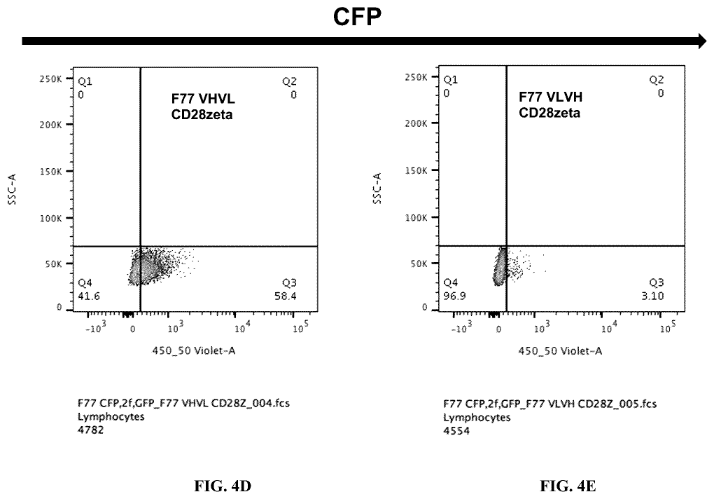

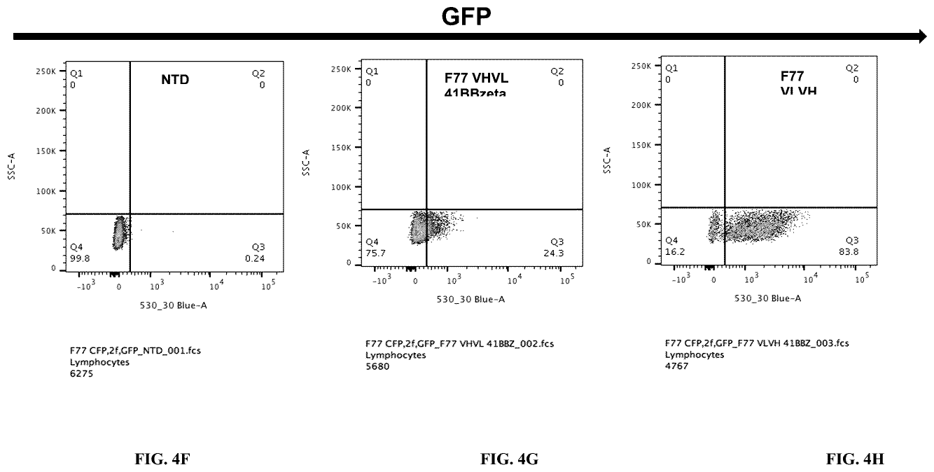

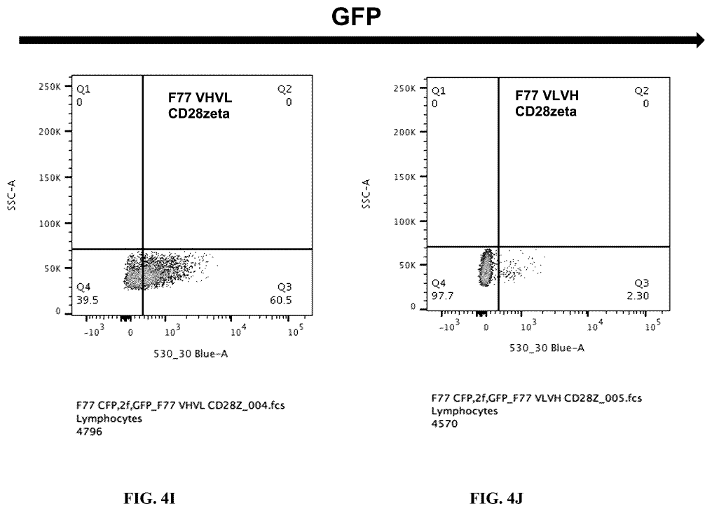

Out of the four constructs, F77 scFv surface expression on CART cells in F77 VLVH 41BBzeta was found to be 84% and in F77 VHVL CD28zeta was found to be 59% when analyzed for CFP fluorescence on 450/50 violet channel. The same two constructs showed good expression, 84% and 61%, respectively when analyzed for GFP fluorescence on 530/30 channel on BD LSRFortessa C as shown in FIGS. 4A-4J. 1×10⁶ cells were washed with 2 ml 1×DPBS and analyzed by flow cytometry on BD LSRFortessa C. NTD represents non transduced T cell without any CAR (control).

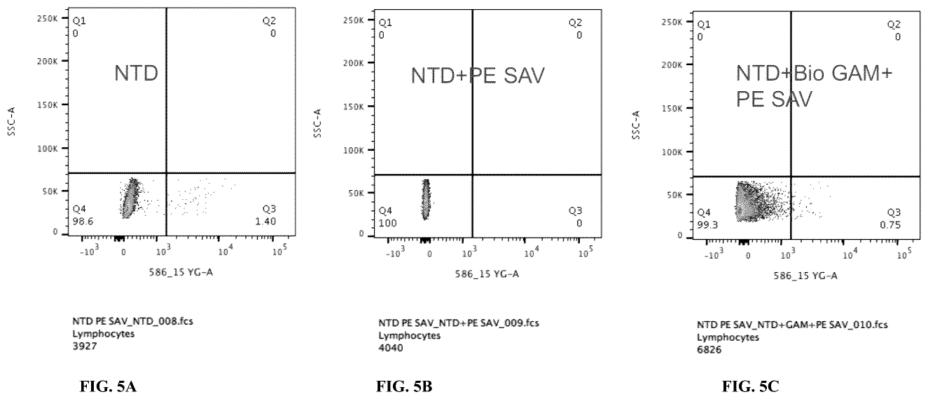

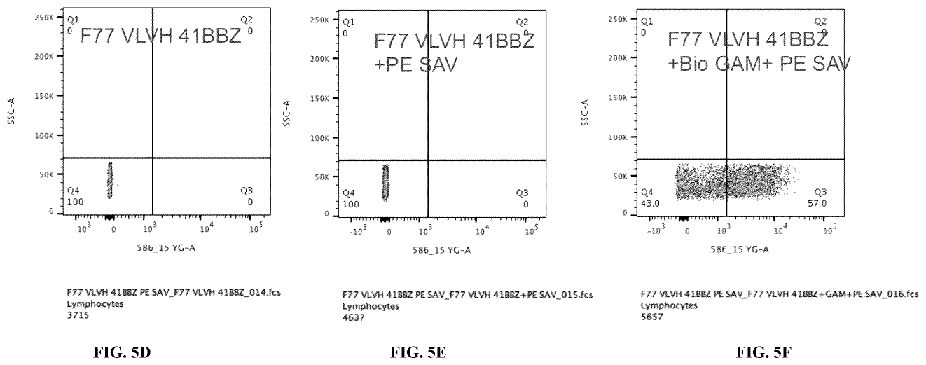

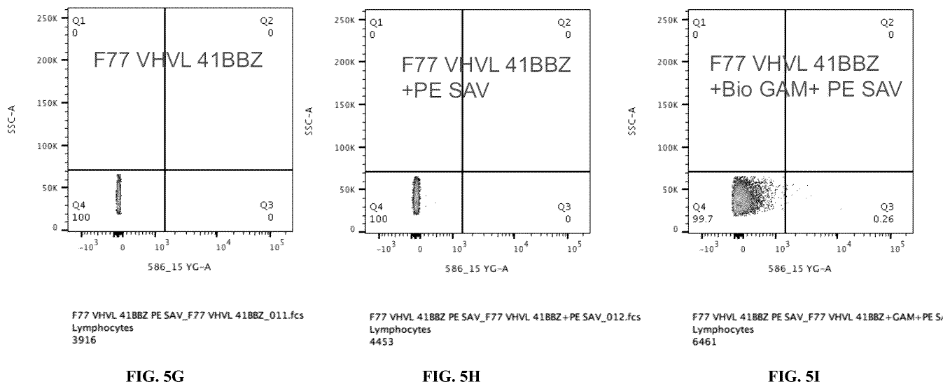

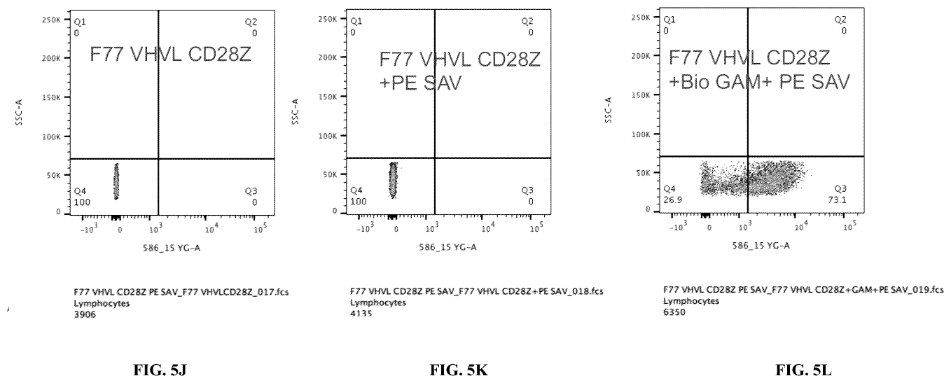

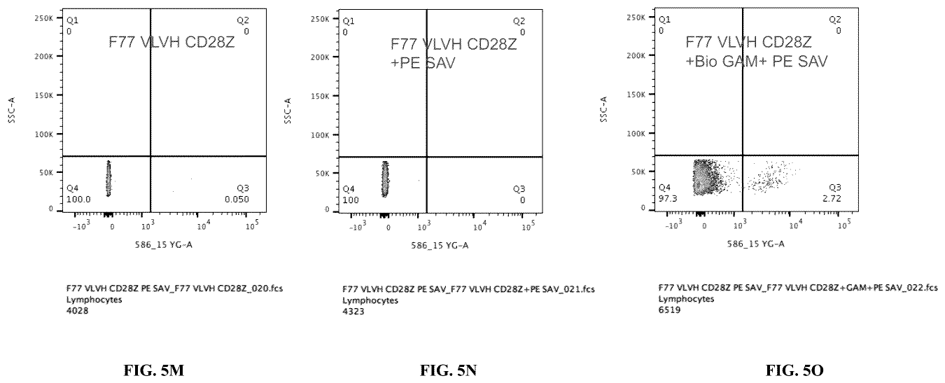

At the same time, these CART cells were also stained by two step staining using Biotin-SP-conjugated AffiniPure Goat Anti Mouse IgG F(ab′2) (Jackson; Cat No. 115-065-072) followed by PE streptavidin (BD Biosciences; Cat No. 554061) and analyzed by flow cytometry on BD LSRFortessa C. NTD is non transduced T cell control. In this case also, only two of four constructs, F77 VLVH 41BBzeta (57%) and F77 VHVL CD28zeta (73%) showed good surface expression as shown in FIGS. 5A-5O. 1×10⁶ CART cells were washed with 2 ml 1×DPBS and stained with Biotin-SP-conjugated AffiniPure Goat Anti Mouse IgG F(ab′2) (Jackson; Cat No. 115-065-072) followed by PE streptavidin (BD Biosciences; Cat No. 554061) and analyzed by flow cytometry on BD LSRFortessa C. NTD is non transduced T cells without any CAR.

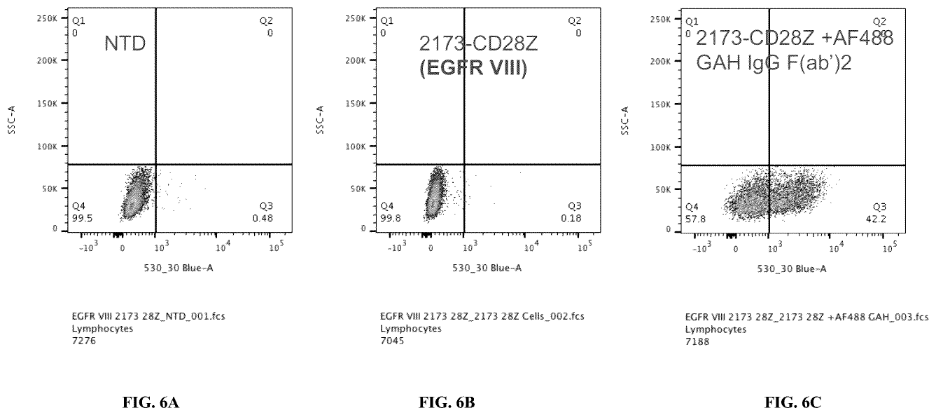

Similarly, 2173-CD28zeta CART cells were stained with Alexa Fluor 488 AffiniPure Goat Anti Human IgG F(ab′2) (Jackson; Cat No. 109-545-006) and analyzed by flow cytometry on BD LSRFortessa C as shown in FIGS. 6A-6C. Expression of EGFR VIII on CART cells was found to be about 42%. 1×10⁶ CART cells were washed with 2 ml 1×DPBS, stained, and analyzed by flow cytometry on BD LSRFortessa C. NTD is non transduced T cells without any CAR.

### Analysis of Cytokine Production

After checking for surface expression by flow cytometry, all F77 CART cells were analyzed by Human IFN-gamma sandwich ELISA assay using Human IFN-gamma DuoSet ELISA kit (R&D systems; Cat No. DY285B-05B) to assess the level of released natural hIFN-gamma from CART cells. For this assay, effectors (CART cells—NTD, F77VHVL 41BBzeta, F77VLVH 41BBzeta, F77VHVL CD28zeta, F77VLVH CD28zeta and 2173-CD28zeta) and target cells (PC3 GFP, PC3 CR GFP, PC3 FUT1KO GFP, PC3 CD44KO GFP, RWPE-1 GFP, RWPE-2 GFP and U87 VIII GFP cells) were incubated overnight at an E:T ratio of 10:3. E:T ratio corresponds to Effector cell (CART cell) to target cancer cell ratio. The samples were diluted 1:5 the following day and tested in a sandwich ELISA assay using Human IFN-gamma DuoSet ELISA kit. The results are shown in FIG. 7 and Table 4. 96 well ELISA plate was coated with 2 μg/ml of Mouse anti-human IFN-gamma capture antibody. Effector cell and target cell mix was added over the capture antibody coated wells and incubated at RT for 2 hrs. The wells were washed followed by addition of Biotinylated mouse anti-human IFN-gamma detection antibody for 1 hr at RT. The bound sample was detected by Streptavidin-HRP followed by addition of colored substrate. The reaction was terminated using stop solution and optical density was measured at 450 nm.

TABLE 4
Human IFN-gamma release from F77 CART cells.

| | | SAMPLE 2 | SAMPLE 3 | SAMPLE 4 | SAMPLE 5 | SAMPLE 6 |
|---|---|---|---|---|---|---|
| | SAMPLE 1 | F77 VHVL | F77 VLVH | F77 VHVL | F77 VLVH | 2173-28Zeta |
| Target cells | NTD | 41BBZeta | 41BBZeta | CD28Zeta | CD28Zeta | (EGFR VIII) |
| PC3 GFP | 0.0 | 0.0 | 5633.0 | 1301.1 | 85.2 | 0.0 |
| PC3 CR GFP | 1.1 | 0.0 | 5572.7 | 2264.8 | 147.7 | 0.0 |
| PC3 Fut1 KO GFP | 0.0 | 8.0 | 71.6 | 0.0 | 0.0 | 0.0 |
| PC3 CD44 KO GFP | 0.0 | 4.5 | 6143.2 | 4798.9 | 667.0 | 0.0 |
| RWPE-1 GFP | 0.0 | 0.0 | 1411.4 | 29.5 | 2.3 | 0.0 |
| RWPE-2 GFP | 8.0 | 14.8 | 5665.9 | 5739.8 | 1088.6 | 60.2 |
| U87 VIII GFP | 48.9 | 15.9 | 71.6 | 14.8 | 3.4 | 4753.4 |

Among the four F77 CARs tested, only two, F77 VLVH 41BBzeta and F77 VHVL CD28zeta showed good release in terms of human IFN-gamma release. Some hIFN-gamma release was seen with RWPE-1 GFP cells. These cells have some minimal level of F77 expression and are not completely F77 negative. 2173-28zeta CAR is EGFR-VIII CAR. This mutant EGFR did not work with prostate cancer cells but showed good human IFN-gamma release with U87 VIII GFP target cells. EGFR VIII CAR served as a negative control for the assay.

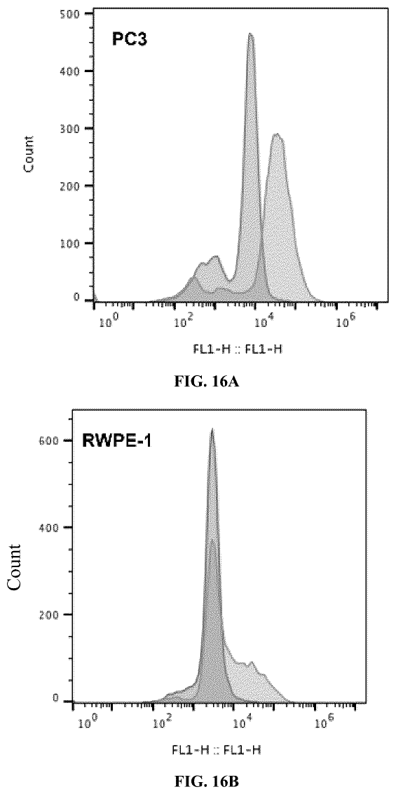

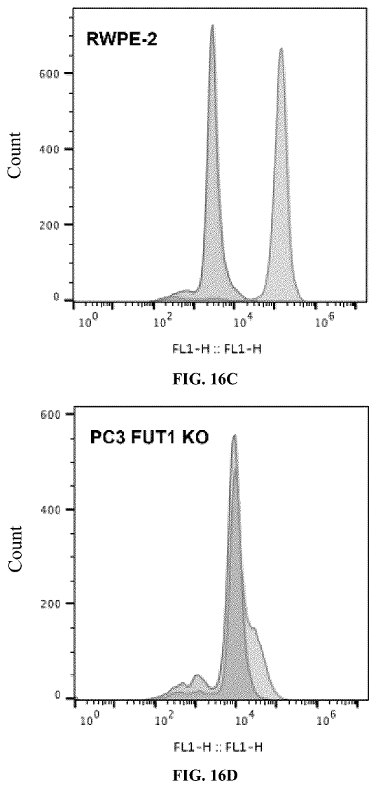

Flow cytometric analysis using the murine IgG3 mAb F77 55.10 revealed that its targeting antigen was expressed at a high level on the surface of PC3 prostate cancer cells (FIG. 16A). mAb F77 55.10 also bound to a small population of the non-tumorigenic human prostate epithelial cell line RWPE-1 (FIG. 16B) but bound with much greater intensity to tumorigenic RWPE-2 cells that were derived from RWPE-1 after transfection with the constitutively active K-ras oncogene (FIG. 16C). K-ras transfection has been previously shown to upregulate F77 antigen levels and these results are in accordance with our previously reported data. FUT1 and one of GCNT1, GCNT2 or GCNT 3 are essential enzymes for F77-specific O-linked glycosylation in prostate cancer cells. As expected, knockout of FUT1 in PC3 (PC3 FUT1 KO) dramatically decreased mAb F77 55.10 binding (FIG. 16D).

Significant IFN γ secretion was observed when co-culturing T cells expressing F77-specific CARs (F77 VLVH 4-1BBzeta or F77 VHVL CD28zeta) with target cells expressing the F77 antigen as determined by flow cytometry with the mAb F77 55.10 described above. Importantly, no secretion of IFN γ was observed when co-cultured with PC3 cells possessing a FUT1 knockout (PC3 FUT1 KO) or U87EGFRvIII GFP cells, a glioma cell line expressing the EGFRvIII antigen, but not the F77 antigen (FIG. 7). The level of IFN 7 correlated with CAR expression as IFN γ levels were undetectable or very low in the supernatant of co-cultures of F77 VHVL 4-1BBzeta and F77 VLVH CD28zeta CAR T cells (no F77 CAR expression was determined by flow cytometry) with PC3 GFP and RWPE-2 GFP target cells. Some level of IFN γ secretion was also observed in the supernatant of co-cultures with F77 VLVH 4-1BBzeta CAR T cells with RWPE-1 GFP target cells. This is likely due to limited expression of F77 antigen on RWPE-1 cells as assessed by the binding study with mAb F77 55.10 (FIG. 16B). Considering the F77 CAR expression on transduced T cells and levels of human IFN γ secretion in co-culture assay, only F77 VLVH 4-1BBzeta and F77 VHVL CD28zeta CARs were further evaluated for their ability to mediate T cell cytotoxicity.

eSight Assay Using xCELLigence Platform

Surface expression analysis by flow cytometry and human IFN-gamma release ELISA assay confirmed the functionality of F77 VHVL 41BBzeta and F77 VLVHCD28 zeta CARs. These two CARs were tested for cytotoxicity on prostate cancer cells using eSight assay on xCELLigence platform.

Six targets and four effector cells were used for this assay. After measuring background impedance with 50 μl media per well, 10,000 target cells (PC3 GFP, PC3 FUT1 KO GFP, RWPE-1 GFP, RWPE-2 GFP, U87 GFP [cells expressing EGFR wildtype] and U87 VIII GFP [cells expressing EGFR VIII; mutant EGFR] were seeded in E-Plate VIEW 96 plate (Agilent Technologies, Cat. No. 300 601 020) and incubated at 37° C. After proliferating for 23 hrs, CART (Effector) cells were added. T cells (NTD [Non transduced T cells; T cells without any CAR], F77 VLVH 41BBzeta, F77 VHVL CD28zeta and 2173-CD28zeta (EGFR VIII CAR) [negative control for the assay] were varied to achieve E:T ratios of 15:1, 10:1 and 5:1 (duplicates were set up for each ratio). E:T ratio corresponds to Effector cell (CART cell) to target cancer cell ratio. Maximum killing was done with 1% Tween-20. While impedance was measured every 15 min, images were acquired every 2 hrs. Exposure times were as follows: green (300 ms), brightfield (automatically optimized by the eSight software).

eSight assay uses a specialized electronic microplate. Incorporated within the glass bottom of all 96 wells, a gold biosensor array continuously and noninvasively monitors cellular impedance. The adhesion of target cells to these biosensors impedes the flow of a microampere electric current-providing an exquisitely sensitive readout of cell number, cell size, and cell substrate attachment strength. This cellular impedance signal is recorded at a user-defined temporal frequency (every minute, once per hour, etc.), and is reported using a unitless parameter called Cell Index. The CAR T cells that are subsequently added to the wells are nonadherent and therefore do not contribute to the impedance signal. The CAR T cell-induced biochemical and cellular changes (cell rounding, detachment, lysis) that occur in the target cells are detected as a progressive drop in the impedance signal.

Impedance is calculated by % cytolysis.

$$\% cy tolysis = [ 1 - Normalized CI treatment / Normalized CI target only ] \times 100.$$

$$( CI = Cell = Index )$$

Using PC3 GFP target cells, both F77 VLVH 41BBzeta and F77 VHVL CD28zeta CART cells showed >90% killing while only 10-20% killing was seen with 2173-CD28zeta CART (EGFR VIII CAR; Negative control). In case of PC3 FUT1 KO GFP target cells (F77 negative cell line), both F77 VLVH 41BBzeta and F77 VHVL CD28zeta CART cells showed minimal killing nearly equivalent to NTD and 2173-CD28zeta CAR (EGFR VIII CAR; Negative control). This served as an excellent control cell line for the assay wherein absence of F77 antigen, there was no significant killing by F77 CART cells.

With RWPE-1 GFP target cells, both F77 VLVH 41BBzeta and F77 VHVL CD28zeta CART cells showed 20-30% killing at 10:1 and 5:1 E:T ratios while 60% and 40% killing was observed at 15:1 E:T ratio. With RWPE-2 GFP target cells, both F77 VLVH 41BBzeta and F77 VHVL CD28zeta CART cells showed about 100% killing. Tumorigenic RWPE-2 cells were derived from non-tumorigenic RWPE-1 after transfection with the constitutively active K-ras oncogene.

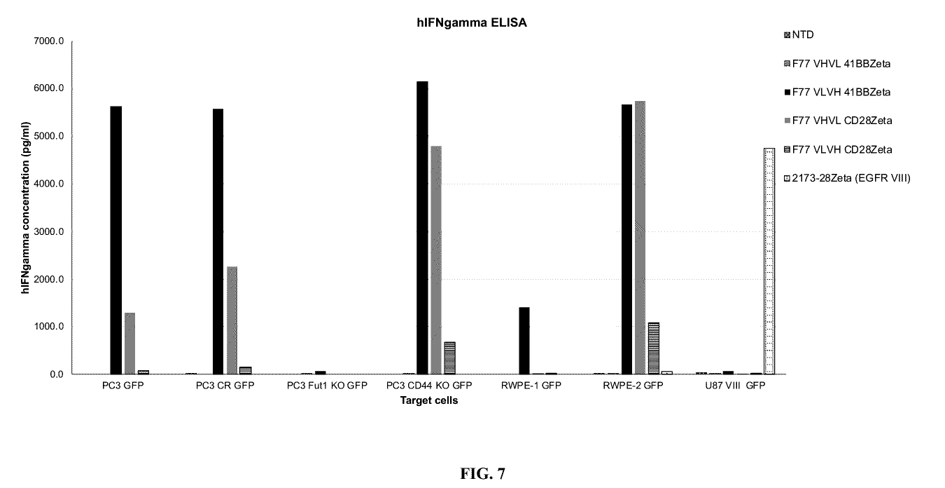

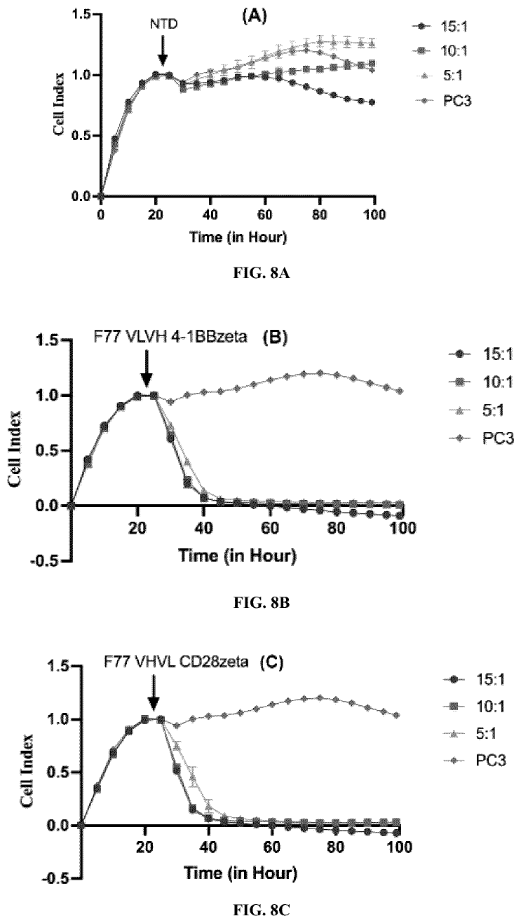

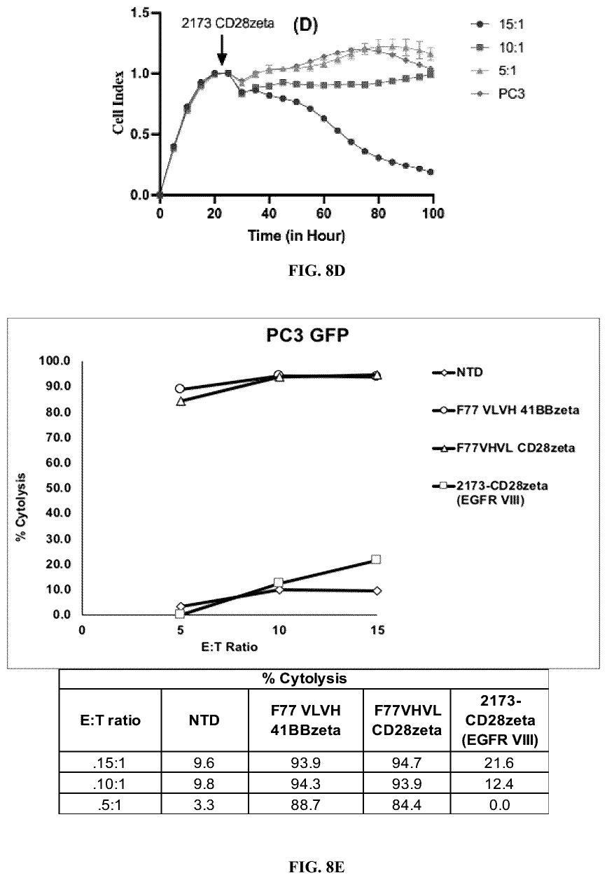

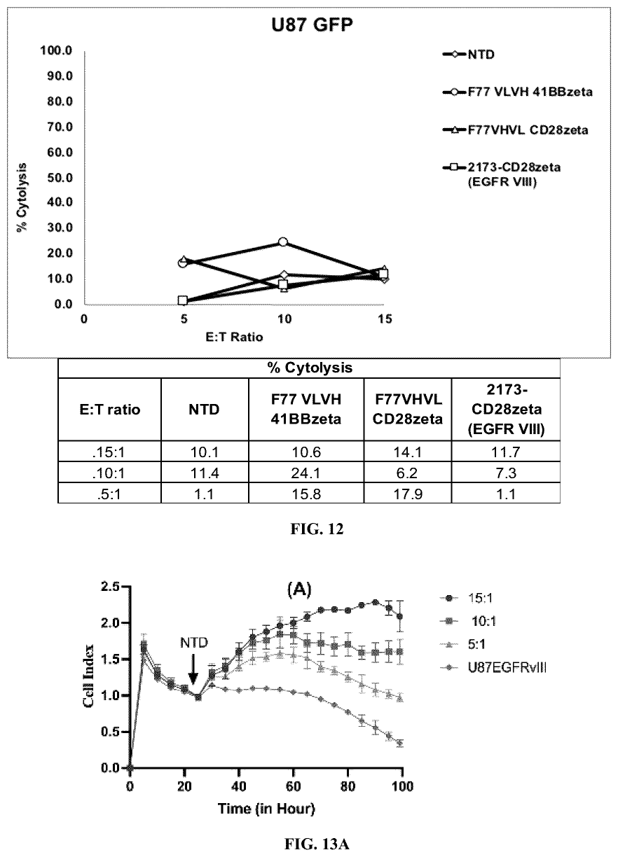

With U87 GFP target cells, both F77 VLVH 41BBzeta and F77 VHVL CD28zeta CART cells showed killing equivalent to NTD and 2173-CD28zeta CAR (negative control). With U87 VIII GFP target cells, both F77 VLVH 41BBzeta and F77 VHVL CD28zeta CART cells showed 0% killing as expected while 2173-CD28zeta CART cells (EGFR VIII CAR) showed 100% killing. The results are shown in FIGS. 8A-13E. 10,000 target cells were seeded per well and T cells were varied to achieve E:T ratios of 15:1, 10:1 and 5:1. Killing was calculated as % cytolysis using Impedance data.

PC3 cells co-cultured with negative control T cells (not transduced with a CAR or NTD) showed no decrease in CI values or cytolysis (FIGS. 8A and 8E). The addition of F77 VLVH 4-1BBzeta and F77 VHVL CD28zeta to PC3 cells caused an immediate and time dependent decrease in CI. 16 hours post addition of F77 effector cells, complete cytolysis of PC3 target cells was observed (FIGS. 8B and 8C). The decrease in CI after addition of effector cells directly correlates with the target cell viability and can be converted to percent cytolysis using formula described in materials and methods section. Both F77 VLVH 4-1BBzeta and F77 VHVL CD28zeta CAR T cells show ˜95% cytolysis indicating complete lysis of PC3 cells at 40 hrs (16 hr after addition of CAR T cells, T=16 hr) at different E:T ratios (FIG. 8E).

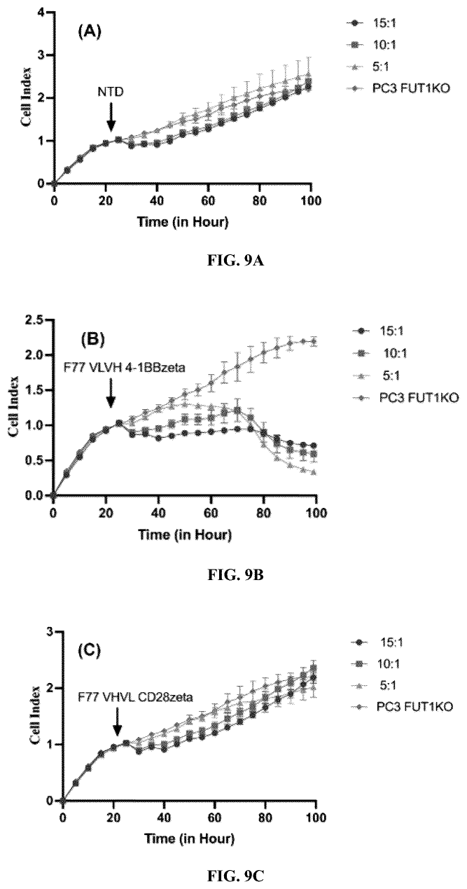

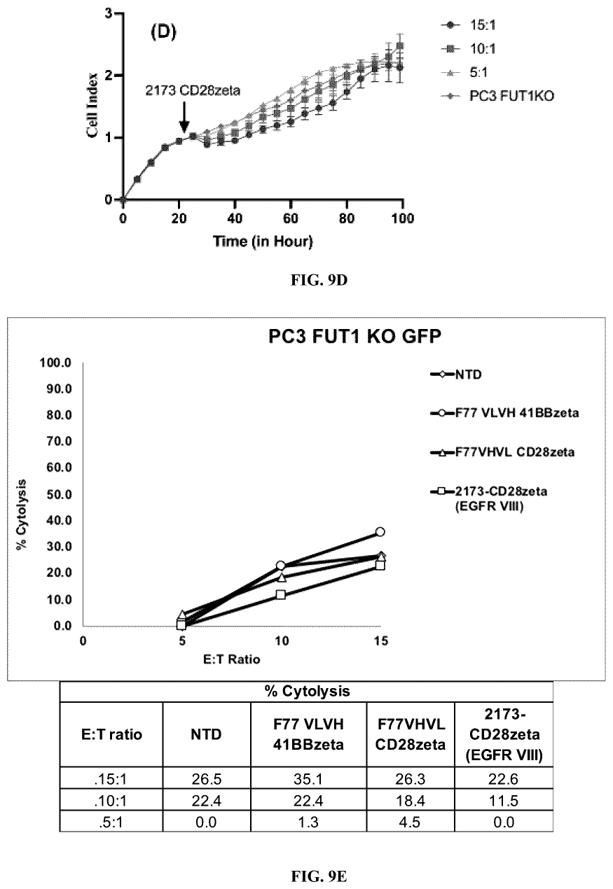

The addition of any of the four effector cells; NTD, F77 VLVH 4-1BBzeta, F77 VHVL CD28zeta or 2173 CD28zeta to PC3 FUT1 KO cells does not lead to any significant decrease in CI values (FIGS. 9A-9D) demonstrating absence of target cell cytolysis when F77 antigen is not expressed on the target cells (FIG. 9E). It has previously been reported that FUT1 enzyme is required for biosynthesis of Fucα1→2-Gal linkage and is essential for F77 antigen expression in mammalian cells. Further knockout of FUT1 dramatically decreased mAb F77 binding activity in PC3 FUT1 KO cells (FIGS. 16A-16D). These previous findings correlate with no significant cytolysis of PC3 FUT1 KO cells upon addition of F77 CAR T cells (FIGS. 9A-9E).

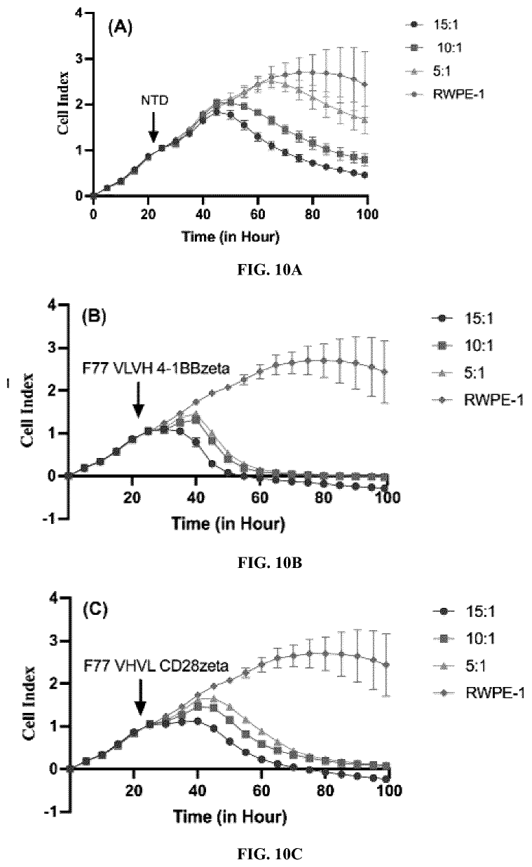

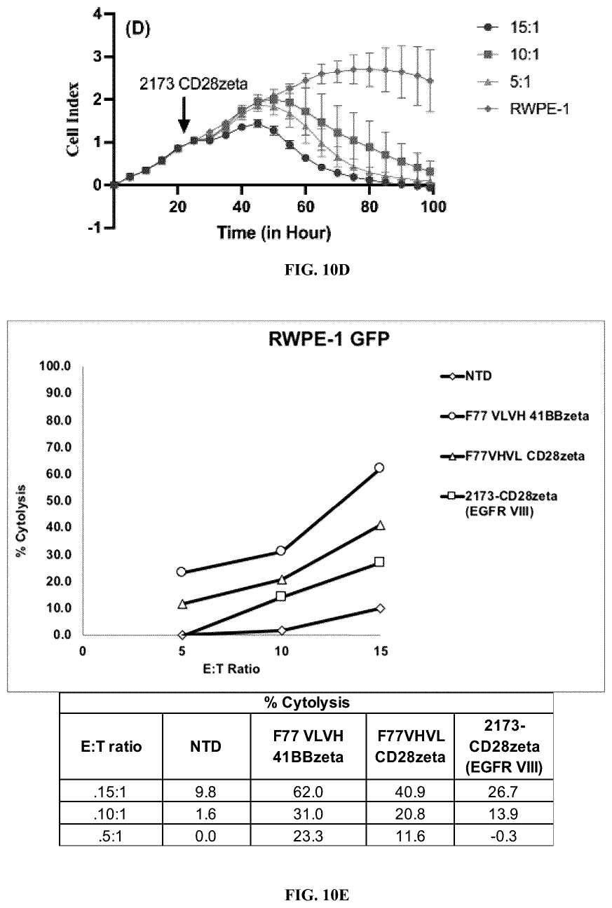

The addition of F77 VLVH 4-1BBzeta and F77 VHVL CD28zeta effector cells to RWPE-1 target cells lead to a slow decrease in CI values (FIGS. 10B and 10C) with detectable levels of target cell cytolysis at high E:T ratio ratios tested (FIGS. 10A-10E). RWPE-1 are benign prostate epithelial cells but show some cytotoxicity with F77 effector cells. This is attributed to the limited expression of F77 antigen on RWPE-1 cell surface as described above by flow cytometry (FIGS. 16A-16D). This also correlates with some level of IFNγ secretion which is observed in the supernatant of co-cultures with F77 VLVH 4-1BBzeta CAR-T cells with RWPE-1 GFP cells (FIG. 7). As expected, no cytolysis was observed after addition of T cells without any CAR (NTD) (FIGS. 10A and 10E).

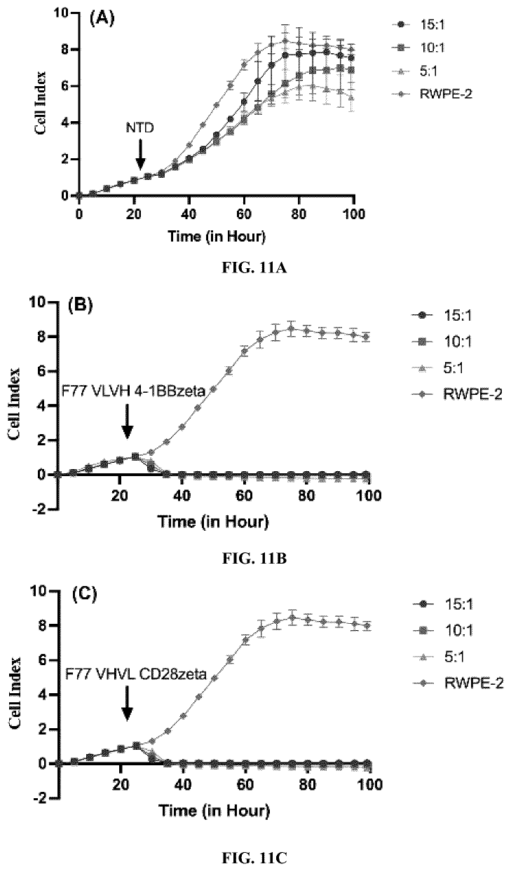

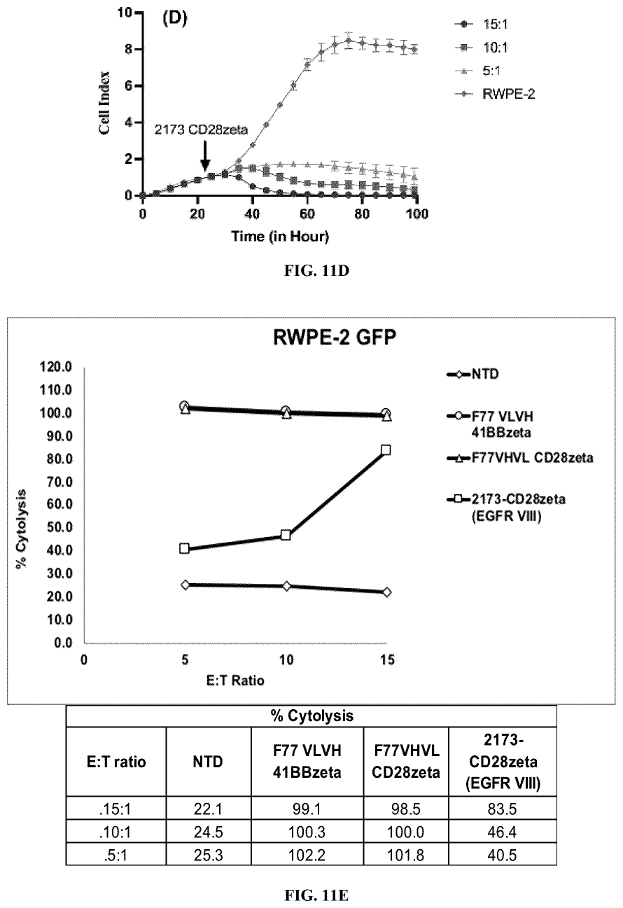

Like PC3 cells, addition of F77 VLVH 4-1BBzeta and F77 VHVL CD28zeta CAR T cells to RWPE-2 cells lead to an immediate drop in CI values indicating complete lysis of target cells at all E:T ratios tested (FIGS. 11B, 11C and 11E). RWPE-2 cells express a high density of F77 antigen as described above by flow cytometry (FIGS. 16A-16D) and K-ras transfection has been previously shown to upregulate F77 antigen levels which correlates with the high percent cytolysis values obtained upon addition of F77 CAR transduced T cells. The addition of T cells without any CAR (NTD) to RWPE-2 cells does not lead to any noticeable decrease in CI values and no significant cytolysis was seen (FIGS. 11A and 11E).

PC3 cells express wildtype EGFR and not the mutant EGFRvIII targeted by 2173 CD28zeta CAR T cells so the addition of these effector cells to PC3 target cells does not lead to any significant target cell cytolysis (FIGS. 8D and 8E). The same effector cells with RWPE-1 target cells show minimal reduction in CI at later time points and low degree of cytolysis at higher E:T ratios tested indicating the absence of EGFRvIII (FIGS. 10D and 10E). On the other hand, RWPE-2 cells show significant reduction in CI values and cytolysis upon addition of 2173 CD28zeta effector cells (FIGS. 11D and 11E) which correlates to the high tumorigenic potential of prostate epithelial cell line RWPE-2 that was derived from RWPE-1 after transfection with constitutively active K-ras oncogene.

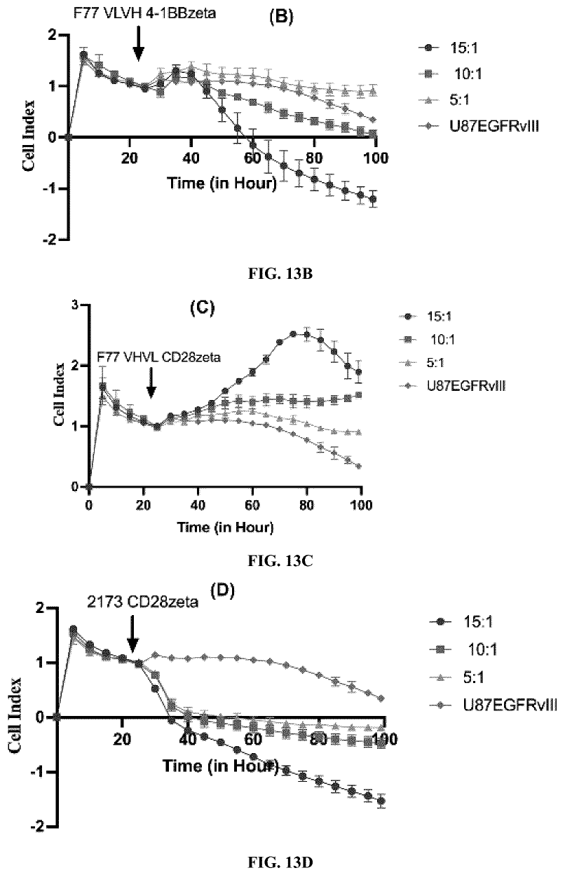

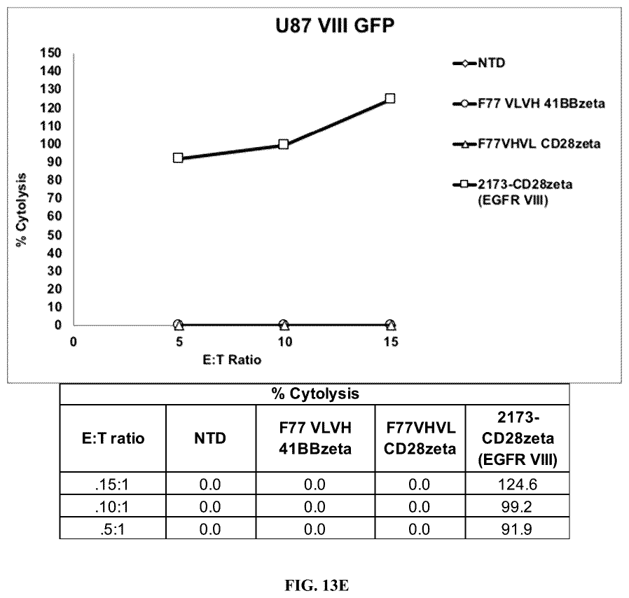

In case of U87EGFRvIII cells which express EGFRvIII but not the F77 antigen, there is no significant decrease in CI values after addition of F77 CAR T cells and thus no cytolysis is observed (FIGS. 13B and 13C) but the addition of 2173-CD28zeta CAR T cells leads to an immediate and time dependent decrease in CI values accompanied by complete cell lysis at all E:T ratios tested (FIGS. 11D and 11E). Like other target cells, the addition of T cells without any CAR (NTD) to U87EGFRvIII cells did not lead to any noticeable decrease in CI values and no cytolysis was seen (FIGS. 13A and 13E).

In short, F77 VLVH 4-1BBzeta and F77 VHVL CD28zeta transduced T cells showed time-dependent elimination of target cells expressing a high density of the F77 target antigen (PC3 and RWPE-2 cells). As expected, there was no effect on cells with low or no F77 antigen (RWPE-1, PC3 FUT1 KO and U87 EGFRvIII) supporting the specificity of F77 CAR T cells for the F77 antigen. Non-transduced T cells and EGFRvIII-specific CAR T cells showed no activity against the F77 antigen expressing target cells, but the latter effectively killed U87EGFRvIII cells.

### Outcome

The above results showed that both F77 VHVL 41BBzeta and F77 VHVL CD 28zeta CARs effectively targeted prostate tumors expressing the F77 antigen.

### In Vivo Evaluation of Efficacy of F77 VLVH 4-1BBzeta and F77 VHVL CD28zeta CARs

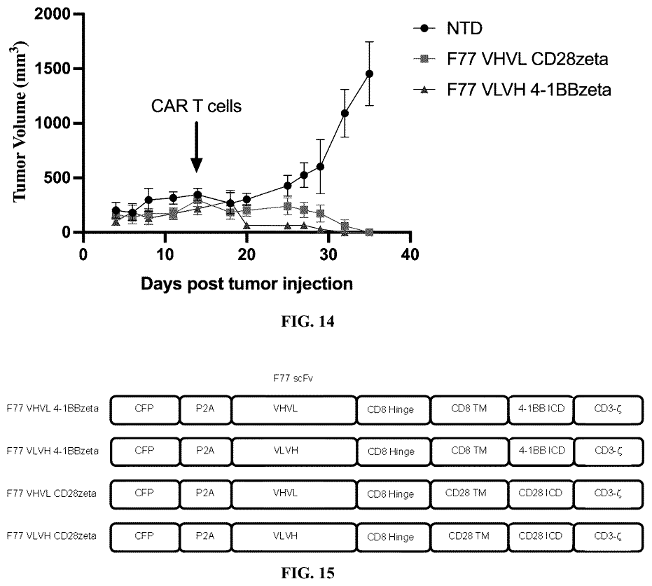

Based upon the ability of F77 VLVH 4-1BBzeta and F77 VHVL CD28zeta CARs to mediate both cytokine secretion and antigen-dependent cellular cytotoxicity in vitro, the ability of these CAR T cells to control tumor growth in vivo was evaluated. PC3 cells expressing GFP were implanted subcutaneously in NSG mice and one of the three effector cells, NTD (T cells without any CAR), F77 VLVH 4-1BBzeta and F77 VHVL CD28zeta were infused intratumorally after 14 days of tumor implantation. Treatment with F77 effector cells inhibited PC3 tumor growth (FIG. 14). Six days post addition of CAR T cells decrease in the size of PC3 tumors was observed followed by complete suppression of tumors by day 35 (21 days after addition of CAR T cells). Both F77 VLVH 4-1BBzeta and F77 VHVL CD28zeta effector T cells were able to completely inhibit tumor growth in PC3 tumor model whereas NTD cells show no appreciable control of the tumor.

Antigen characterization revealed F77 antigen to be expressed on glycan structures composed of Fucα1→2Galβ1→4GlcNAcβ1→6Gal/GalNAc. Also, FUT1 and at least one glutaminyl (N-acetyl) transferase were found to be essential for F77 antigen expression on mammalian cells. mAb F77 55.10 was selected over mAb F77 129 due to a variation in the glycosylation site in complementarity determining region 2 (CDR2) of the heavy chain (data not shown here), even though their respective DNA sequences are almost identical.

In this example, CARs were designed that incorporated the variable regions of the anti-prostate mAb F77 55.10. T cells transduced with F77 CAR-encoding lentiviral vectors were found to be effective in in vitro functional tests and suppressed PC3 tumors in an in vivo tumor xenograft model. Both F77 CARs proved to be extremely efficient at complete cytolysis of PC3 and RWPE-2 prostate cancer cells. Some degree of cytolysis was also seen in benign prostate cancer cells RWPE-1 which correlated with the limited expression of F77 antigen on its cell surface as assessed by flow cytometry with mAb F77 55.10. Importantly, knockdown of FUT1 gene drastically reduced mAb F77 binding activity in PC3 FUT1 KO cells, which is in accordance with previous observations that F77 CARs do not cause substantial cytolysis of PC3 FUT1KO target cells. The absence of any cytolysis of U87EGFRvIII target cells also supported that the F77 CARs are highly selective for target cells expressing the F77 antigen. These F77 CAR T cells were also tested in a human RBC blocking assay in comparison to mAb F77 55.10 where failure of F77 mediated killing of RBCs was observed with F77 CAR T cells while mAb F77 55.10 blocked blood types O and A but not blood type B (data not shown here). Also, mAb F77 55.10 was found to be capable of inducing PC3 tumor cell apoptosis in an in vitro assay (data not shown here).

Overall, the present example describes the creation and validation of F77 CARs that exclusively target F77 antigen expressed on prostate tumor cells, making it a promising candidate for immunotherapy of prostate cancer. This study is the first ever report that describes a CAR-directed against a carbohydrate antigen for targeting prostate tumors.

### Example 6. F77 Antigen is a Promising Target for Adoptive T Cell Therapy of Prostate Cancer

Adoptive immunotherapy using chimeric antigen receptor (CAR) T cells has made significant success in treating hematological malignancies, paving the way for solid tumors like prostate cancer. However, progress is impeded by a paucity of suitable target antigens. A carbohydrate antigen, F77, is expressed on both androgen-dependent and androgen-independent prostate cancer cells, making it a immunotherapy target. This example describes the generation and evaluation of a second-generation CAR against a carbohydrate antigen on malignant prostate cancer cells. Using a single chain fragment variable (scFv) from an F77-specific mouse monoclonal antibody, a second-generation CARs were created with CD28 and CD137 (4-1BB) costimulatory signals. F77 expressing lentiviral CAR T cells produced cytokines and killed tumor cells in a F77 expression-dependent manner. These F77-specific CAR T cells eradicated prostate tumors in a human xenograft model employing PC3 cells. These findings demonstrated F77 as an immunotherapeutic target for prostate cancer and other malignancies with this aberrant carbohydrate structure.

Abbreviations: Chimeric antigen receptor (CAR), single chain fragment variable (scFv), Fucosyl transferase 1 (FUT1), cyan fluorescent protein (CFP), green fluorescent protein (GFP), Epidermal growth factor receptor variant III (EGFRvIII), non-transduced T cells (NTD), F77 VLVH 4-1BBzeta (F77 CAR1), F77 VHVL CD28zeta (F77 CAR2), 2173 CD28zeta (Control CAR), Effector-target ratio (E:T), interferon gamma (IFNγ).

Prostate cancer is the second most diagnosed cancer among men and the fourth most common type of cancer worldwide. It is the fifth leading cause of death from cancer among men, with an estimated 1.4 million new cases and 375,000 deaths. Current treatment options for prostate cancer include surgical removal, radiation therapy, hormonal therapy, or chemotherapy. Even with the recent advances, most patients with metastatic disease ultimately develop therapeutic resistance progressing to an androgen-independent state.

The chimeric antigen receptor T (CAR T) cell therapy in the treatment of hematological malignant tumors as an adoptive immunotherapy achieved significant advances. In its most fundamental form, a CAR is a synthetic receptor consisting of an immunotyrosine-based activation motif (ITAM)-containing signaling domain for T cell activation and an antigen-binding domain, which is commonly an antibody-derived single chain antibody fragment (scFv) to direct specificity. First-generation CARs consist of the ITAM-containing CD3 (cytoplasmic domain alone whereas second-generation CARs include an additional co-stimulatory signaling domain from CD28, 4-1BB (CD137), OX-40, CD27 or inducible costimulatory (ICOS).Third generation and later generation CARs combine more than one co-stimulatory signaling domain (e.g., 4-1BB and CD28) along with other signals such as cytokines like IL-18. Upon CAR mediated detection of tumor cells, CAR T cells get activated, destroy tumor cells, proliferate, and establish long-term memory.

T cell immunotherapy offers an approach for treatment of prostate cancer by targeting tissue-specific antigens expressed on malignant prostate cells. Altered carbohydrates of cell surface glycoproteins and glycolipids on malignant cells are among one such highly coveted biomarkers for disease diagnosis. Monoclonal antibodies against carbohydrate structures on malignant cells have previously been reported to target tumor cells through antibody-dependent cellular cytotoxicity (ADCC) and antibody-dependent phagocytosis by macrophages. One such cytotoxic antibody is the murine mAb F77 129, IgG3 subtype kappa, raised against the PC3 human adenocarcinoma recognizing a unique carbohydrate antigen, highly restricted to both androgen-independent (PC3, PC3-MM2, DU145) and androgen-dependent (LnCaP) prostate cancer cell surfaces. Immunohistological studies with mAb F77 129 showed staining of >90% of primary prostate cancers, with only limited focal staining of benign prostate tissues, and 29 of 34 metastatic human prostate cancer specimens also stained positive. Normal or tumor tissues of human colon, kidney, cervix, pancreas, lung, skin, and bladder were not stained with mAb F77 129 confirming that the antigen recognized by mAb F77 is highly restricted to prostate and is overexpressed in prostate tumors. This antibody significantly inhibits androgen-independent tumor growth in both PC3 and Du145 mouse tumor xenograft models, with no activity against an F77-negative xenograft and employs a CD44-dependent mechanism to induce tumor cell apoptosis. A novel sandwich ELISA-based assay employing mAb F77 129 identified a glycosylated form of CD44 from both prostate cancer cells and sera from men with prostate cancer substantiating the potential therapeutic utility mAb F77 129 for detection of prostate cancer. The F77 antigen has been found to be expressed on blood group O (H), specifically on a 6-linked branch of a poly-N-acetyllactosamine backbone. mAb F77 129 is capable of binding to blood group A and B analogs as well, although with lower intensities compared to its binding to blood group H. The close association between the F77 antigen and prostate cancers is attributed to the combination of increased blood group H expression and upregulated branching enzymes. In prostate cancers, there is an elevated expression of blood group H antigen, which is facilitated by the upregulation of branching enzymes involved in its synthesis. The expression of the F77 antigen in mammalian cells requires co-transfections with glycosyltransferase genes. Specifically, the glycosyltransferase gene FUT1 and one of the genes GCNT1, GCNT2, or GCNT3 are necessary for F77 antigen expression.

mAb F77 55.10 is another murine mAb F77, IgG3 subtype kappa, derived from the same fusion as mAb F77 129 described above. Like mAb F77 129, mAb F77 55.10 shows high binding to PC3 prostate carcinoma.

Herein, the mAb F77 55.10 scFv was used to create second-generation CAR targeting F77 carbohydrate antigen, which besides the CD3 (chain contains the signaling domains of 4-1BB or CD28. In this example, it is demonstrated that primary T lymphocytes isolated from the peripheral blood of healthy volunteers expressing F77 scFv CAR induce in vitro and in vivo tumor reactivity against highly aggressive androgen-independent PC3 prostate cancer cells.

### Materials and Methods

### Target Cell Lines and Reagents

The PC3 prostate cancer cell line was originally isolated from a bone metastasis in a 62-year-old Caucasian with grade IV adenocarcinoma of the prostate. PC3 FUT1KO cell line derived from PC3 and was generated in-house by Fucosyl transferase 1 (FUT1) CRISPR knockout. The prostate cancer cell lines were maintained in Dulbecco's modified Eagle's medium (DMEM) supplemented with 10% fetal bovine serum (FBS, HyClone), penicillin and streptomycin. RWPE-1 is a normal human prostate epithelial cell line immortalized by human papilloma virus 18 (HPV-18). RWPE-2 cells were derived from RWPE-1 cells by transformation with K-ras using the Kirsten murine sarcoma virus (Ki-MuSV). Both RWPE-1 and RWPE-2 cell lines were maintained in Keratinocyte Serum Free Medium (KSFM, Gibco) supplemented with bovine pituitary extract (BPE) and human recombinant epidermal growth factor (EGF). U87 is an aggressive human glioblastoma cell line derived from malignant gliomas of a male patient. U87EGFRvIII, a U87 glioblastoma cell line overexpressing epidermal growth factor receptor variant III (EGFRvIII) was generated in-house at Dr. Michael C. Milone's laboratory (Perelman School of Medicine of the University of Pennsylvania) and was maintained in RPMI-1640 medium supplemented with 10% fetal bovine serum (FBS, HyClone), 1% HEPES, 1% penicillin and streptomycin. All cell lines except PC3 FUT1KO and U87EGFRvIII were obtained from the American Type Culture Collection. Cell-line authentication was performed by the University of Pennsylvania Genetics Core based on criteria established by the International Cell Line Authentication Committee. Short-tandem-repeat profiling revealed that these cell lines were above the 90% match threshold. All cells were cultured in standard conditions using a humidified incubator with 5% CO₂ at 37° C. All target cells expressing GFP were created by transducing the respective parental cell line with a lentiviral vector encoding GFP. Following transduction, the cells were expanded and sorted by fluorescence activated cell sorting (FACS) using BD FACS Jazz system (BD Biosciences) to obtain a population of >95% GFP positive cells.

mAb F77 55.10 was purified from culture supernatant of F77 55.10 hybridoma cells as described previously. PE-conjugated mouse mAb EGFR 528 (sc120) for determination of EGFR surface expression was obtained Santa Cruz Biotechnology. Lentiviral vectors with 4-1BB or CD28 signaling domains for cloning of F77 scFv and lentiviruses encoding EGFRvIII CAR (2173 CD28zeta CAR or Control CAR) and GFP (pELNS-GFP-T2A-CBG; for generating target cell lines expressing GFP) were provided by Dr. Michael C. Milone, Perelman School of Medicine of the University of Pennsylvania. Male NSG (NOD-SCID-Il2rg−/−) mice were used for in vivo studies.

### Generation of Lentiviral Vectors Encoding F77 CARs

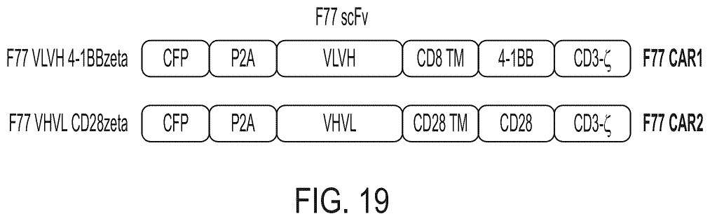

Sequencing of mAb F77 55.10 was performed by Yurogen Biosytems LLC (Worcester, USA). The heavy chain and light chain variable-region sequences of the antibody were used to design single chain fragment variable (scFv) with the following pattern: heavy chain variable region-linker-light chain variable region (VHVL) and light chain variable region-linker-heavy chain variable region (VLVH). The linker had the following amino acid sequence: GGGGSGGGGSSGGGS. DNA encoding F77 scFv was codon optimized and synthesized by GENEWIZ with BamHI and NheI restriction sites. These DNA sequences were cloned into a bicistronic, 3^{rd} generation lentiviral vector to generate a vector that expresses a CAR with either a 4-1BB or CD28 immuno-costimulatory domain as previously described and cyan fluorescent protein (CFP) separated by a P2A ribosomal skip sequence. F77 VLVH 4-1BBzeta and F77 VHVL CD28zeta are referred to as F77 CAR1 and F77 CAR2, respectively. Schematic representation of CAR constructs is shown in FIG. 19.

### Lentiviral Supernatant Production

To produce supernatant containing replication defective lentiviruses, a third-generation lentiviral transfer plasmid encoding appropriate CAR was transfected into HEK293T cells (ATCC) using Lipofectamine 2000 (Invitrogen) with three packaging plasmids: pRSV-rev (encoding rev), pMDLgal/pRRE (encoding gag and pol), pMDG.1 (encoding the vesicular stomatitis virus envelope VSV-G). The supernatant was harvested 24 h and 48 h post-transfection, filtered using a 0.45 μM syringe filter, centrifuged at 12,000 rpm for 24 h, and the concentrated pellet was resuspended in collecting medium, aliquoted, and stored at −80° C.

### T Cell Transductions and Expansion

Primary human T cells from healthy volunteer donors were obtained from the Human Immunology Core at the University of Pennsylvania. Informed consent was obtained from all participants before collection. All methods and experimental procedures were approved by the University of Pennsylvania Institutional Review Board. Bulk T cells were isolated by negative selection using RosetteSep T cell enrichment cocktail (StemCell Technologies, Vancouver, BC, Canada). T cells were activated using anti-CD3/anti-CD28 coated Dynabeads (Thermo Fisher Scientific, Waltham, MA, USA) at a bead:cell ratio of 3:1 and cultured in RPMI1640 with 10% fetal bovine serum (FBS), 10 mM HEPES, and 1% penicillin/streptomycin. 24 h after activation, the T cells were transduced with lentiviruses at a MOI of 3. T cells were then expanded in media containing 100 IU/mL IL-2 (Proleukin) until resting for 10⁻¹⁴ days, with a cellular volume of approximately 400 fL, prior to cryopreservation. Rested and frozen T cells were thawed and washed with media prior to functional in vitro and in vivo assays.

### Staining and Flow Cytometric Analysis

Surface expression of F77 antigen was compared among different prostate cancer cell lines (PC3, PC3 FUT1KO, RWPE-1, RWPE-2), and glioblastoma cell lines (U87 and U87EGFRvIII) by staining with purified mAb F77 55.10 using the same method as previously described. Briefly, cells were trypsinized, washed twice with PBS, and then fixed with 4% paraformaldehyde in PBS at room temperature for 15 min. Following washing with PBS, cells were blocked with 10% goat serum for 30 min. Approximately 0.5-1×10⁶ cells were resuspended in 100 μl FACS buffer (1% BSA/PBS solution) and stained with 5 μg of the purified mAb F77 55.10. Cells were thoroughly mixed and incubated at 4° C. for an hour. Thereafter, the cells were washed twice with FACS buffer, and subsequently they were incubated for an hour at 4° C. with FITC-labeled goat anti-mouse antibody or APC-labeled goat anti-mouse antibody (1:100; Jackson ImmunoResearch Laboratories, Inc.). The cells were then washed twice with FACS buffer followed by flow cytometry analysis using BD Accuri or LSRFortessa C (BD Biosciences) and analyzed with FlowJo X 10.5.3 (Tree Star, Ashland OR).

For CAR expression, T cells were washed with 2 ml room temperature PBS and the CFP expression of the F77 CAR molecule on the surface of T cells was detected with the violet laser. Control (2173 CD28zeta; EGFRvIII) CAR T cells were stained with Alexa Fluor 488 AffiniPure Goat Anti Human IgG F(ab′₂) (Jackson ImmunoResearch Laboratories, Inc.). Flow cytometry analysis was performed using LSRFortessa C (BD Biosciences) and analyzed with FlowJo X 10.5.3 (Tree Star, Ashland OR).

Surface expression of EGFR was compared among different prostate cancer cell lines; PC3, PC3 FUT1KO, RWPE-1 and RWPE-2 using U87 as a control cell line by staining with PE-conjugated mouse mAb EGFR 528 (sc120). Cells were trypsinized and washed twice with PBS. Approximately 0.5-1×10⁶ cells were resuspended in 100 μl PBS and stained with 1 μg of PE-conjugated mouse mAb EGFR 528 (sc120) at 4° C. for an hour. The cells were washed twice with PBS and flow cytometry analysis was performed using LSRFortessa C (BD Biosciences) and analyzed with FlowJo X 10.5.3 (Tree Star, Ashland OR).

### Interferon (IFN) γ Release ELISA

The CAR T cells were co-cultured with target cells at an effector-target (E:T) ratio of 10:3 in flat bottom 96-well plates for 16 h at 37° C. and 5% CO₂. The supernatants were collected, diluted 1:5 and subjected to IFNγ detection by sandwich ELISA assay using Human IFN-gamma DuoSet ELISA kit (R&D) according to manufacturer's instructions. Briefly, a 96-well ELISA plate was coated with 2 μg/ml of mouse anti-human IFNγ capture antibody. Supernatant from co-cultures of effector cell and target cell was added over the capture antibody coated wells and incubated at RT for 2 h. The wells were washed followed by addition of biotinylated mouse anti-human IFNγ detection antibody for 1 h at RT. The bound sample was detected by streptavidin-HRP followed by addition of colored substrate. The reaction was terminated using a stop solution and optical density was measured at 450 nm in a multimode reader (Emax Plus Microplate reader, Molecular devices). Each individual experiment was performed in duplicates.

Real time potency assessment using xCELLigence real-time cell analysis (RTCA)

The xCELLigence RTCA instrument (Agilent Technologies) was utilized for in vitro cytotoxicity experiments. The RTCA system utilizes cellular impedance readout to monitor real-time changes in target cell number, cell size, and cell-substrate attachment strength using a parameter called Cell Index (CI) to reflect the viability of target cancer cells. Briefly, 50 μl of target cancer cell culturing media was added to each well of E-Plate VIEW 96 plate (Agilent Technologies) and the background impedance was measured. Adherent GFP labeled target cells were seeded in the E-Plate VIEW 96 plate at a density of 10,000 cells/well (PC3, PC3 FUT1KO, RWPE-1, RWPE-2 and U87EGFRvIII (cells expressing EGFRvIII; mutant EGFR) in a volume of 50 μl and incubated at 37° C., 5% CO₂. After monitoring cell proliferation for 24 h, CAR T (effector) cells (NTD [Non transduced T cells; T cells without any CAR], F77 CAR1, F77 CAR2 and control CAR [negative CAR control for the prostate cancer cell lines]) were added at several E:T ratios of 15:1, 10:1 and 5:1 in duplicates. E:T ratio corresponds to effector cell (CAR T cell) to target cancer cell ratio. A full lysis control was included by adding 1% tween-20 to target cells. Impedance was measured every 15 min. Exposure times were as follows: green (300 ms), brightfield (automatically optimized by the eSight software). Changes in impedance were reported as Cell Index (CI) and Normalized Cell Index (NCI). Impedance-based measurements of the normalized cell index were collected every 15 min, which were determined by measuring of the impedance of current across the plate caused by target cell adherence. Impedance was calculated by % Cytolysis.

$$\% Cytolysis = [ 1 - Normalized CI treatment / Normalized CI target only ] \times 100.$$

$$( CI = Cell Index )$$

### In Vivo Tumor Model

NSG (NOD-SCID-Il2rg−/−) mice were housed in the Xenograft Core Facility at the University of Pennsylvania under pathogen-free conditions in compliance with experimental protocols approved by Institutional Animal Care and Use Committee of the University of Pennsylvania. Six- to eight-week-old NSG mice were injected subcutaneously with 5×10⁶ PC3 cells in 100 μl PBS and matrigel at 50:50 ratio. On day 14 after tumor implantation when tumors reached a volume of 200 mm³, mice were randomly assigned and intratumorally injected with 5×10⁶ CAR T cells: non transduced (NTD; T cells without any CAR) or F77 CAR1 or F77 CAR2 (40-60% transduction efficiency). Tumor volume was measured by caliper using the formula (π/6)×(length)×(width)² at the indicated times (n=5 mice per group).

### F77 Mediated Killing of Red Blood Cells (RBCs)

Healthy adult red cells of blood groups 0, A and B were washed twice with 1×DPBS at 1300×g, 4° C. for 5 min. The washed RBCs were diluted in 1×DPBS and counted using a hemocytometer. Each blood type was diluted to 1% and 3% to get cell counts of 2×10⁷/ml and 6×10⁷/ml, respectively. 20 μl of each dilution corresponding to 0.4×10⁶/ml and 1.2×10⁶/ml, respectively was used for the assay. 5000 CAR T cells/20 μl (NTD, F77 CAR1 or F77 CAR 2) were mixed with 20 μl of RBCs in a 96 well U-bottom plate and incubated for 30 min-1 h. Similarly, several concentrations of mAb F77 55.10 ranging from 78 ng/ml to 5.5 μg/ml were mixed with 20 μl of RBCs in a 96 well U-bottom plate and incubated for 30 min-1 h. The samples were analyzed for F77 mediated killing of RBCs using an inverted microscope.

### Statistical Analysis

Prism v9 (GraphPad Software) was used to plot data and perform statistical analyses. The precise statistical tests used, and significance levels are indicated in the figure legends (* p<0.001 and ** p<0.0001 and ns refers to p>0.05).

### Results

### Surface Expression of F77 Antigen on Prostate Cancer Cells Using mAb F77 55.10

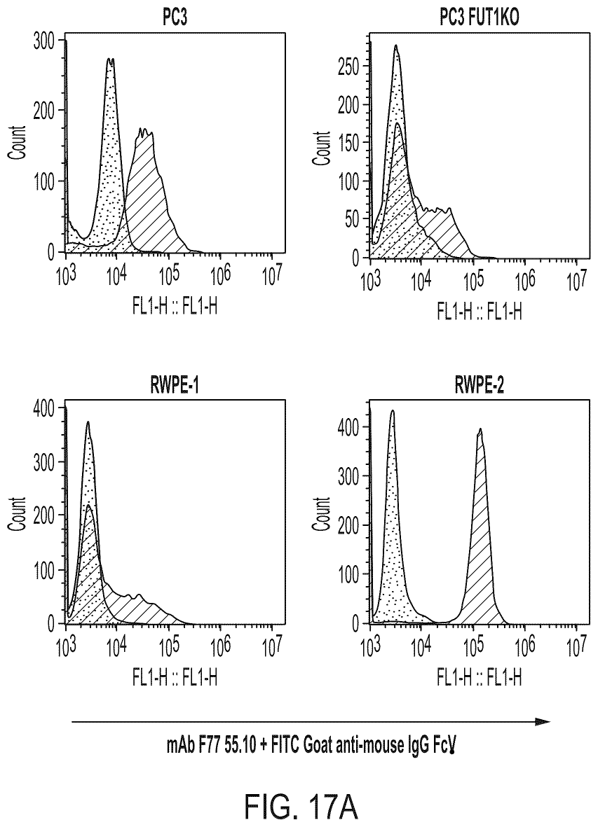

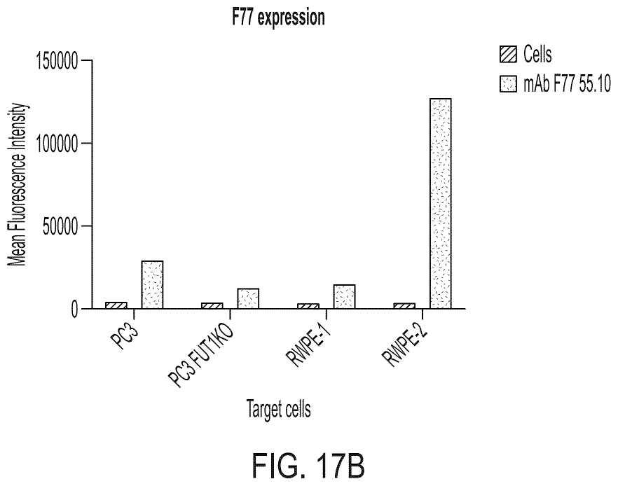

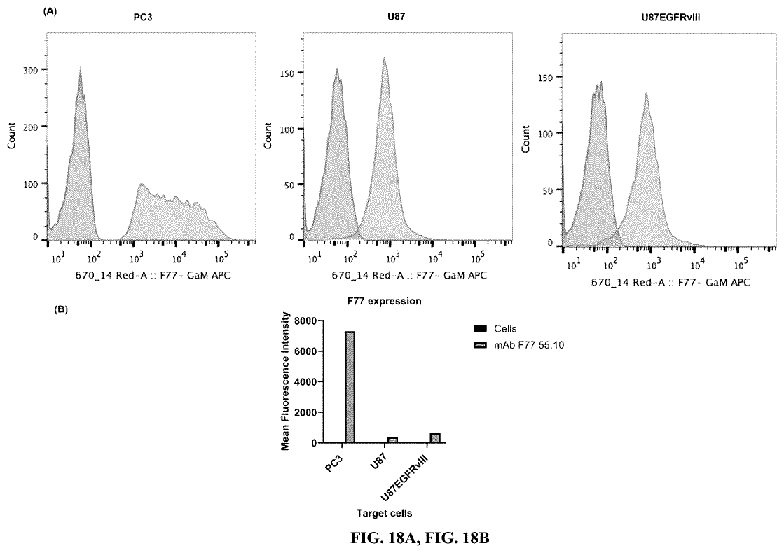

To test the ability of F77 CAR T cells to recognize their cognate antigen, prostate cancer or normal epithelial cells were used with or without genetic modification to modulate F77 expression. Flow cytometric analysis using the murine IgG3 mAb F77 55.10 revealed that its targeting antigen was expressed at a high level on the surface of PC3 prostate cancer cells (FIGS. 17A and 17B). FUT1 and one of GCNT1, GCNT2 or GCNT3 are essential enzymes for F77-specific O-linked glycosylation in prostate cancer cells. As expected, knockout of FUT1 in PC3 (PC3 FUT1KO) dramatically decreased mAb F77 55.10 binding (FIGS. 17A and 17B). mAb F77 55.10 also bound to a small population of the non-tumorigenic human prostate epithelial cell line RWPE-1 (FIGS. 17A and 17B) but bound with much greater intensity to tumorigenic RWPE-2 cells that were derived from RWPE-1 after transfection with the constitutively active K-ras oncogene (FIGS. 17A and 17B). K-ras transfection has been previously shown to upregulate F77 antigen levels and these results were in accordance with the previously reported data. U87EGFRvIII and U87 cell lines exhibited greater than tenfold reduction (11-fold and 18-fold for U87EGFRvIII and U87 cells, respectively) in immunostaining intensity when utilizing monoclonal antibody F77 55.10 in contrast to PC3 cells (FIGS. 18A and 18B).

### Transduced T Cells Efficiently Express F77-Specific CAR Molecules

To evaluate the activity of F77 CAR T cells against prostate tumors, second-generation CARs with an F77 scFv were developed where the VL domain was placed either at the N-terminus or C-terminus of the VH domain to obtain two CARs, F77 CAR1 and F77 CAR2, respectively. To improve signal transduction through the CAR molecule, the intracellular domains of 4-1BB or CD28 were included and fused to the CD3ζ chain (FIG. 19).

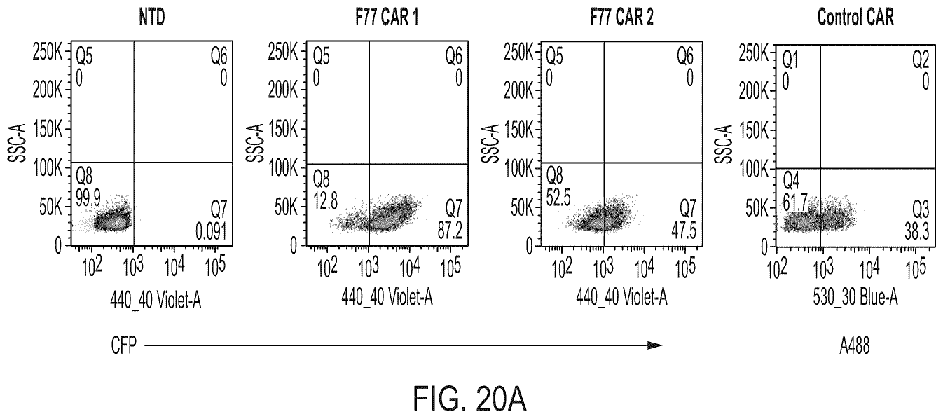

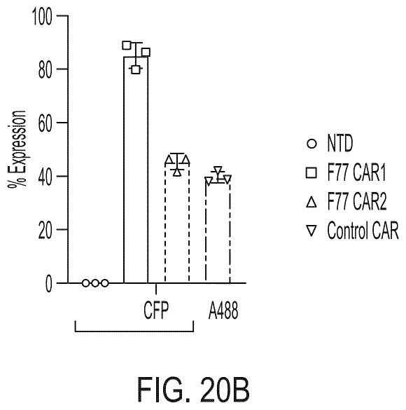

Anti-CD3/anti-CD28 activated primary human T cells isolated from healthy donors were transduced with lentiviral vectors encoding F77-specific CARs with either a 4-1BB or CD28 costimulatory domain. An EGFRvIII specific CAR (2173 CD28zeta) was used as a Control CAR. T cells were efficiently transduced with F77 CAR1 and F77 CAR2 when assessed using CFP expression while expression of control CAR was assessed by surface CAR expression with Alexa Fluor 488 AffiniPure Goat Anti Human IgG F(ab′2) as shown in FIGS. 20A and 20B.

### F77 CAR T Cells Efficiently Secreted INFγ

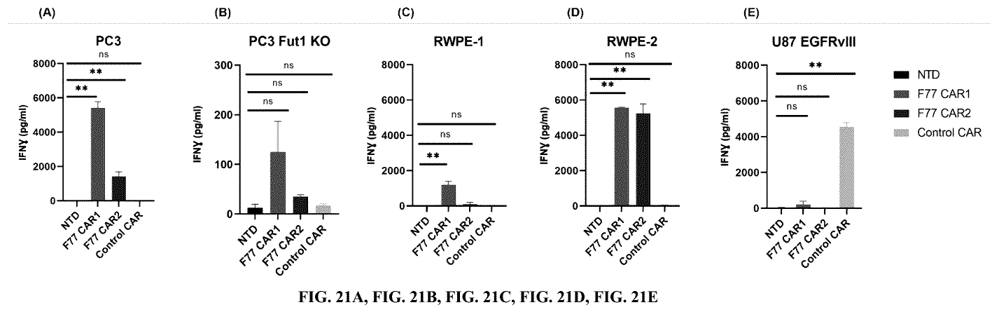

To examine cytokine secretion by CAR T cells, prostate cells or glioblastoma cells were co-cultured with CAR T cells for 16 h. Co-culturing F77-specific CAR-expressing T cells with F77-expressing target cells (PC3 and RWPE-2) led to substantial IFNγ secretion (FIGS. 21A-21E). Importantly, no IFNγ secretion was observed when F77 CAR T cells were co-cultured with U87EGFRvIII cells (a glioma cell line expressing the EGFRvIII antigen but not the F77 antigen) (FIGS. 21A-21E and FIGS. 18A and 18B). A discernable level of IFNγ secretion was also observed in the supernatant of co-cultures with F77 CAR1 T cells with PC3 FUT1KO cells (PC3 cells where FUT1 has been knocked out) and RWPE-1 target cells. This was likely due to limited expression of F77 antigen on both PC3 FUT1 KO and RWPE-1 cells as assessed by the binding study with mAb F77 55.10 (FIGS. 17A and 17B). Considering that both F77 CARs showed similar expression on transduced T cells and both secreted IFNγ in the co-culture assay with PC3 and RWPE-2 target cells, the two F77 CARs were further evaluated for their ability to mediate T cell cytotoxicity.

Monitoring Effector-Mediated Cytolysis of Prostate Cancer Cell Lines Using xCELLigence Real-Time Cell Analysis (RTCA)

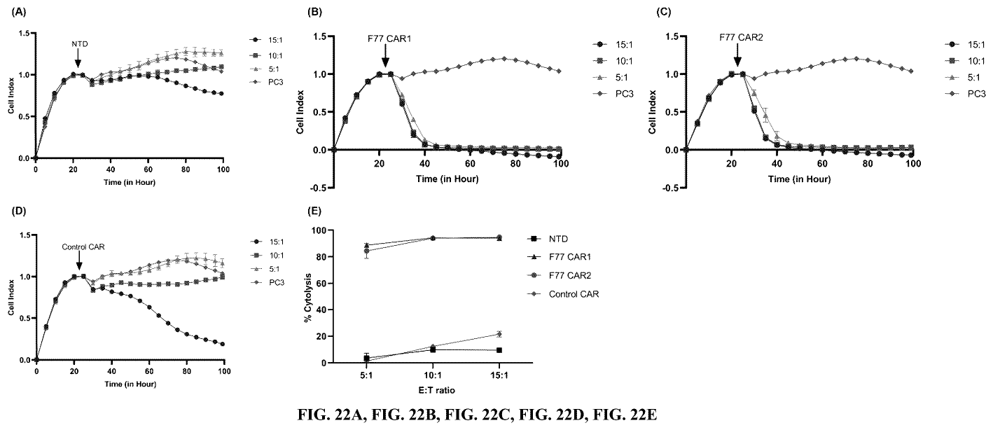

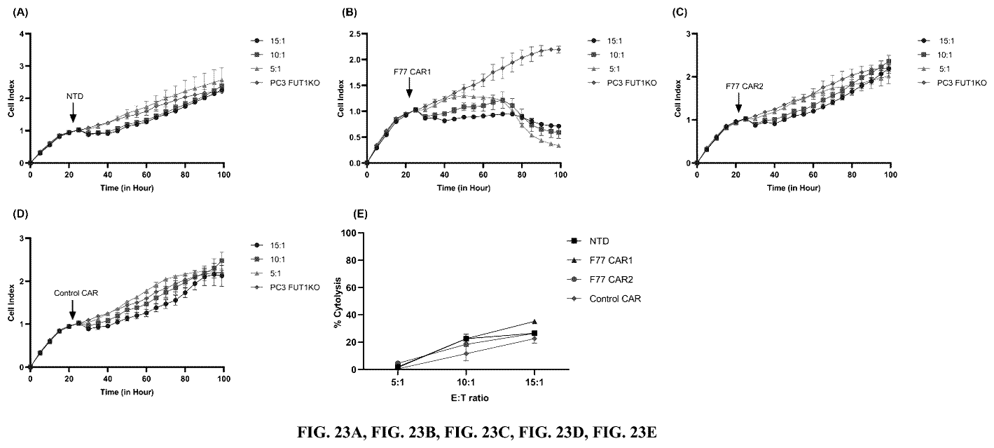

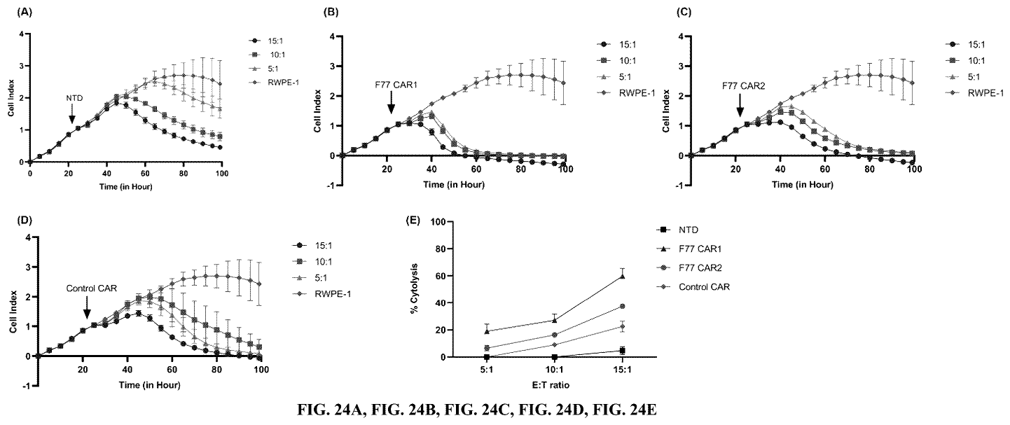

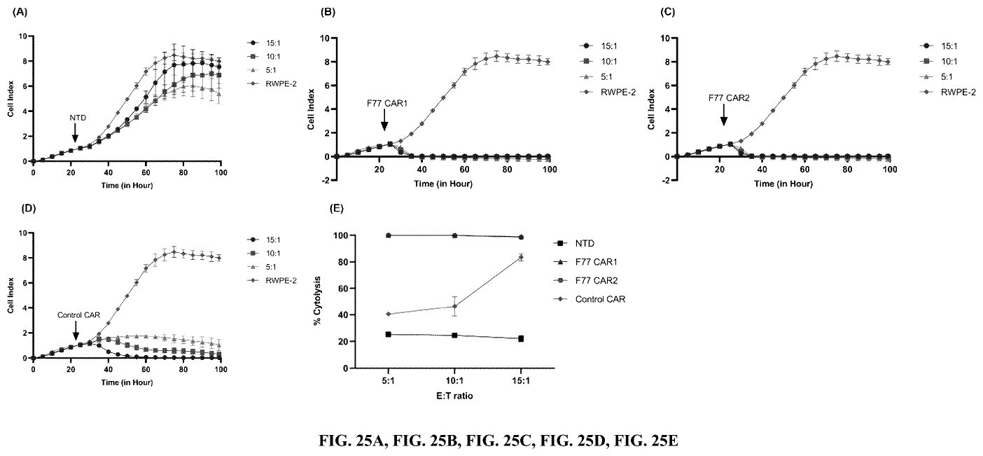

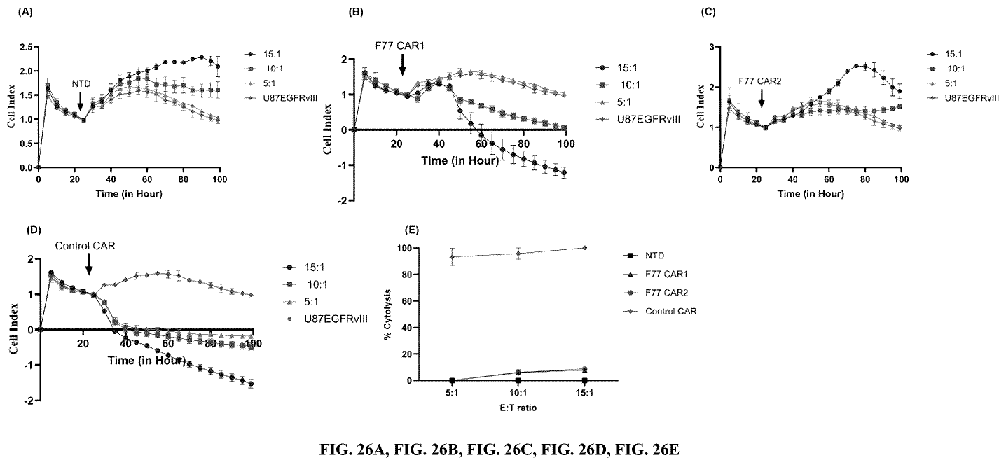

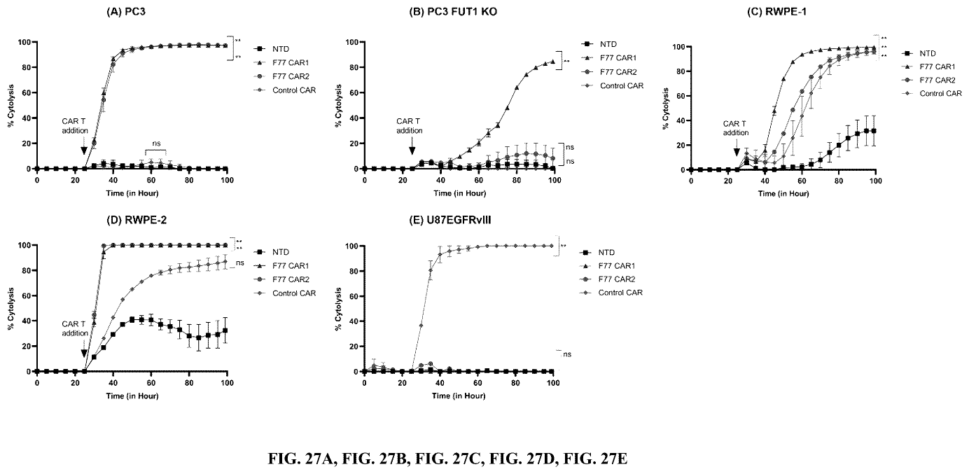

In order to assess the ability of F77 CAR to trigger antigen-dependent cellular cytotoxicity, the same prostate cancer cell lines were used with varying expression of F77 antigen as described above. All prostate cancer target cell lines expressing green fluorescent protein (GFP) were cultured in E-Plate VIEW 96 plates and their proliferation was monitored by the electronic impedance of the plate-sensor electrodes (FIGS. 22A-22E; FIGS. 23A-23E; FIGS. 24A-24E; FIGS. 25A-25E; FIGS. 26A-26E). This enabled detection of target cell attachment to the wells, which was measured as the cell index (CI) value. The decrease in CI after the addition of effector cells directly correlated with the target cell viability and could be converted to percent (%) cytolysis using the formula described in the Materials and Methods section. After 24 h, F77 CAR1, F77 CAR2 and control CAR effector cells were added to the wells at E:T ratios of 15:1, 10:1 and 5:1. PC3 cells co-cultured with negative control T cells (not transduced with a CAR or NTD) showed no decrease in CI values (FIGS. 22A-22E) or % cytolysis (FIG. 27A). As shown in FIGS. 22A-22E, the addition of either of the two F77 CARs to PC3 cells caused an immediate and time dependent decrease in CI and % cytolysis. The addition of F77 effector cells, F77 CAR1 or F77 CAR2 cells induced efficient cytolysis of PC3 target cells (95%; p<0.0001) (FIG. 27A). Both F77 CAR T cells showed increased cytolysis indicating complete lysis of PC3 cells over time at different E:T ratios tested (FIGS. 22A-22E). No cytolysis was observed after addition of control CAR to PC3 cells (FIG. 27A).

The addition of NTD, F77 CAR2 (p=0.8681) or control CAR (p=0.9844) to PC3 FUT1KO cells (where FUT1 has been knocked out) did not lead to decrease in CI values (FIGS. 23A-23E), and did not induce any markedly noticeable % cytolysis (FIG. 27B). It has previously been reported that FUT1 enzyme is required for biosynthesis of Fucα1→2-Gal linkage and is essential for F77 antigen expression in mammalian cells. Thus, knockout of FUT1 dramatically decreased mAb F77 binding to PC3 FUT1KO cells (FIGS. 17A and 17B). However, the residual F77 antigen expression on PC3 FUT1KO cells (FIGS. 17A and 17B) correlated with the level of cytolysis at later time points after F77 CAR1 addition. (p<0.0001, FIG. 27B).

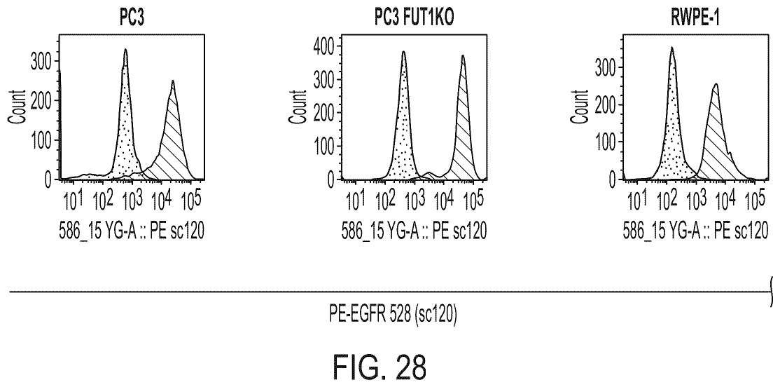

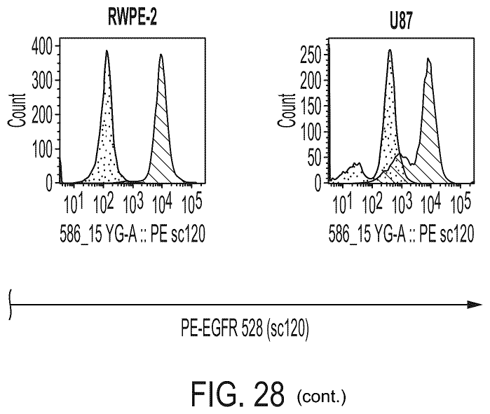

The addition of F77 CAR1, F77 CAR2 or control CAR effector cells to RWPE-1 target cells lead to a slow decrease in CI values (FIGS. 24A-24E) with detectable levels of target cell cytolysis (p<0.0007, p<0.0127, p<0.0371 respectively; FIG. 27C). RWPE-1 are benign prostate epithelial cells but showed some cytotoxicity with F77 effector cells at later time points (FIG. 27C). This could be attributed to the limited expression of F77 antigen and notable levels of EGFR expression on the RWPE-1 cell surface as shown by flow cytometry (FIGS. 17A and 17B and FIG. 28). This also correlated with the scarce level of IFNγ secretion observed in the supernatant of co-cultures of F77 CAR1 T cells and RWPE-1 GFP cells (FIGS. 21A-21E). As expected, no cytolysis was observed after addition of T cells without any CAR (NTD) (FIG. 27C). Over time, there was a reduction in the cell index of RWPE-1 target cells when non-transduced donor T cells were introduced, and this could be attributed to various factors. Competition for essential resources, cytotoxicity from donor T cells releasing molecules like perforin, immune responses triggering apoptosis, and physical contact leading to inhibition which contribute to cell death. Moreover, cytotoxic T cells recognizing foreign antigens in target cells cause cell death. Additionally, prolonged T cell activation leads to apoptosis in both T and target cells. In summary, these factors collectively lead to a decrease in target cell index during extended co-culturing in cytotoxicity assays, as demonstrated in FIGS. 24A-24E. Control CAR T effector cells with RWPE-1 target cells showed minimal reduction in CI at later time points (FIGS. 24A-24E).

K-ras transformed RWPE-2 cells have previously been shown to have upregulated F77 antigen levels which correlated with a reduction in the CI values (FIGS. 25A-25E) and an increase in % cytolysis upon addition of both F77 CAR1 and F77 CAR2 transduced T cells (p<0.0006, FIG. 27D). The addition of T cells without any CAR (NTD) to RWPE-2 cells did not lead to an appreciable decline in CI values and consequently marked cytolysis was not observed (FIG. 27D and FIGS. 25A-25E). RWPE-2 cells showed some reduction in CI values and cytolysis over time upon addition of control effector cells (Control CAR, p=0.0819; FIG. 27D and FIGS. 25A-25E) which correlated to the high tumorigenic potential of prostate epithelial cell line RWPE-2 that was derived from RWPE-1 after transfection with constitutively active K-ras oncogene.

In case of U87EGFRvIII cells which express EGFRvIII but markedly low levels of F77 antigen as compared to PC3 cells as shown in FIGS. 18A and 18B, there was no appreciable decline in CI values after addition of F77 CAR T cells and thus no cytolysis is observed (p=0.991, FIG. 27E, and FIGS. 26A-26E) but the addition of control CAR T cells lead to an immediate and time dependent decrease in CI values accompanied by an efficient and complete target cell lysis at all E:T ratios tested (p<0.0001, FIG. 27E, and FIGS. 26A-26E). This suggested that the EGFR control was unambiguous and other targets were not recognized. Like other target cells, the addition of T cells without any CAR (NTD) to U87EGFRvIII cells did not lead to any noticeable decrease in CI values and no cytolysis was seen (FIG. 27E, FIGS. 26A-26E). Also, all these prostate cell lines with varying levels of F77 antigen also expressed wildtype EGFR, as determined by flow cytometry using PE-conjugated mouse mAb EGFR 528 (sc1-120) (FIG. 28).

Overall, the in vitro cytolysis results demonstrated time-dependent elimination of F77 positive target cells (PC3 and RWPE-2 cells) by F77 CAR transduced T cells. As expected, there was no cytolysis of target cells with no F77 antigen (U87EGFRvIII) supporting the specificity of F77 CAR T cells for the F77 antigen. Non-transduced T cells and EGFRvIII-specific CAR (control CAR) T cells showed no activity against the F77 antigen expressing target cells, but the latter effectively killed U87EGFRvIII cells.

### In Vivo Evaluation of Efficacy of F77 CARs

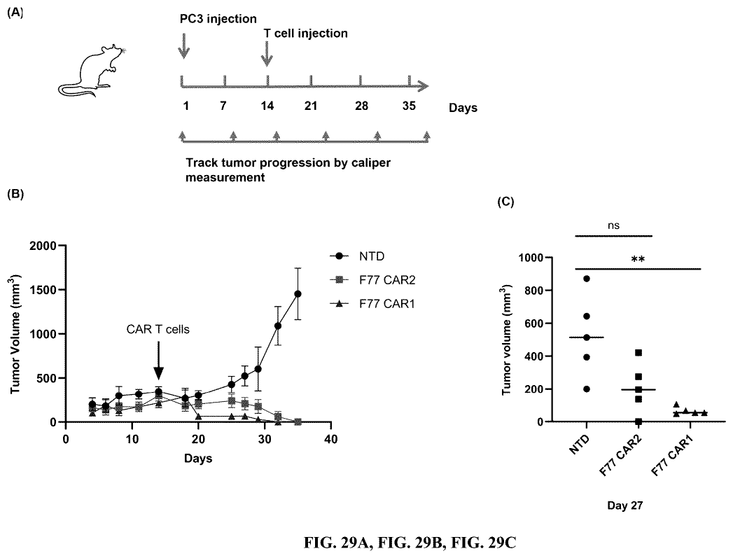

Prior work has shown that 4-1BB costimulation improves function in some settings such as acute leukemia, but in other settings, particularly solid tumors, CD28 costimulation may be superior. Based upon the ability of both F77 CARs to mediate both cytokine secretion and antigen-dependent cellular cytotoxicity in vitro, the ability of these CAR T cells to control tumor growth in vivo was evaluated. PC3 cells expressing GFP were implanted subcutaneously in NSG mice, and 14 days after tumor implantation, the mice were intratumorally infused with NTD (T cells without any CAR), F77 CAR1, or F77 CAR2 (FIG. 29A). Treatment with both F77 effector cells controlled PC3 tumor growth with complete suppression of tumors by day 35 (21 days after addition of CAR T cells) (FIG. 29B). Substantial anti-tumor efficacy was observed at day 27 after infusion of F77 CAR2 (p=0.2691). However, only mice infused with F77 CAR1 showed complete remission of tumors (p=0.0089, FIG. 29C). Mice infused with NTD effector cells did not exhibit any anti-tumor activity. These findings showed that F77 CAR T cells with a 4-1BB costimulatory domain were more effective at completely inhibiting tumor growth in the PC3 tumor model, whereas NTD cells exhibited no measurable tumor control.

### F77 Mediated Killing of Red Blood Cells

The F77 CAR T cells were also tested in a human RBC killing assay in comparison to mAb F77 55.10. CAR T cells (NTD or F77 CAR1 or F77 CAR2) were mixed with either a low (1%) or intermediate (3%) number of various red blood cells representing blood groups O, A, or B. As a reference, multiple concentrations of mAb F77 55.10 were mixed with red blood cells for a duration of 30 minutes, and the subsequent lysis of the erythrocytes was analyzed using an inverted microscope. The results revealed an absence of F77 mediated RBC lysis with the F77 CAR T cells. In contrast, when using mAb F77 55.10, RBC lysis was observed for blood types O and A but not for blood type B, regardless of whether the RBC dilutions were 1% or 3% (as shown in Tables 5 and 6). This is in accordance with the previous data that mAb F77 129 can bind to blood group A and B analogs but with lower intensities compared to its binding to blood group O. In comparison to F77 mAbs which demonstrate characteristics of low titer cold agglutinins, the F77 CAR T cells did not exhibit F77-mediated killing of red blood cells (RBCs).

TABLE 5
F77 mediated killing of RBCs. 5000 CAR T cells (NTD
or F77 CAR1 or F77 CAR2) were mixed with low number
(1%) or intermediate number (3%) of different red
blood cells (blood group O, A, or B).

| | Blood type | | |
|---|---|---|---|
| CAR T cells | O | A | B |
| NTD | − | − | − |
| F77 CAR1 | − | − | − |
| F77 CAR2 | − | − | − |
| PBS | − | − | − |

(+) represents killing of RBCs and (−) represents absence of killing of RBCs.

TABLE 6
mAb F77 55.10 mediated killing of RBCs. Different concentrations
of mAb F77 55.10 were mixed with red blood cells (blood
group O, A or B) for 30 min and killing of the erythrocytes
was analyzed under an inverted microscope.

| | |
|---|---|
| Concentration of | Blood type |

| | | | |
|---|---|---|---|
| mAb F77 55.10 | O | A | B |

| | | | | |
|---|---|---|---|---|
| 5.5 | μg/ml | + | + | − |
| 2.5 | μg/ml | − | − | − |
| 1.25 | μg/ml | − | − | − |
| 625 | ng/ml | − | − | − |
| 313 | ng/ml | − | − | − |
| 156 | ng/ml | − | − | − |
| 78 | ng/ml | − | − | − |
| 0 | ng/ml | − | − | − |

(+) represents killing of RBCs and (−) represents absence of killing of RBCs.

Despite being an excellent target for immunotherapies, there is a lack of curative therapies for prostate cancer, especially in androgen-independent metastatic prostate cancer. Therapeutic vaccines have been widely tested in patients with advanced stage prostate cancer but exhibit little efficacy in clinical settings. To date, Sipuleucel-T is the only FDA approved vaccine for treatment of metastatic prostate cancer, while others were found to be less efficient. CAR T cells constitute an innovative and potent new class of therapeutic agents that offer promise for curative responses in patients with solid malignancies.

Numerous Chimeric Antigen Receptors (CARs) have been engineered to target specific antigens associated with prostate cancer, including Prostate Stem Cell Antigen (PSCA), Prostate-Specific Membrane Antigen (PSMA), and Epithelial Cell Adhesion Molecule (EpCAM)], as well as Six-Transmembrane Epithelial Antigen of Prostate 1 (STEAP1). Many of these CAR therapies have progressed to human clinical trials. Notably, STEAP1 CAR T cell therapy has been employed in conjunction with localized IL2 immunotherapy to target advanced prostate cancer, demonstrating an augmentation in anti-tumor efficacy. Additionally, ongoing clinical trials, such as NCT03873805 and NCT02744287, are evaluating CAR T therapies targeting the PSCA antigen, while another trial, JNJ-75229414, focuses on CAR T therapy directed against KLK2 for metastatic castration-resistant prostate cancer (NCT05022849). In spite of the fact that PSMA and PSCA are desirable targets, both antigens are also expressed in several normal tissues other than the prostate with some toxicity, including fatal toxicity observed in a phase I PSMA-specific CAR T cells trial with unclear etiology.

Cancer carbohydrate antigens or tumor associated carbohydrate antigens (TACAs) have proved to be surprisingly potent immunogens because of their abundant expression at the cell surface as compared to normal cells. Anti-cancer vaccines targeting TACAs have been developed with new antigen architectures, carrier moieties, and immune activation components like adjuvants and cytotoxic T cell epitopes. CAR T cells and bispecific antibodies targeting the two TACAs-gangliosides GD2 and glycoprotein mucin-1 (MUC1) have made recent strides in the treatment of solid malignancies. Several tumor specific monoclonal antibodies have been developed, and the majority have been directed against cell surface carbohydrate antigens such as glycolipids and glycoproteins. Anti-carbohydrate mAb F77 was identified as a novel prostate-specific antibody recognizing glycolipids as well as O-linked glycosylation on proteins in both androgen-independent and androgen-dependent prostate cancer cells. Antigen characterization revealed F77 antigen to be expressed on glycan structures composed of Fucα1→2Galβ1→4GlcNAcβ1→6Gal/GalNAc. Also, FUT1 and at least one glutaminyl (N-acetyl) transferase were found to be essential for F77 antigen expression on mammalian cells. Anti-carbohydrate mAb F77 55.10 was derived from the same fusion as mAb F77 129 and shows a similar binding profile to prostate carcinomas like mAb F77 129. mAb F77 55.10 was selected over mAb F77 129 due to a variation in the glycosylation site in complementarity determining region 2 (CDR2) of the heavy chain, even though their respective DNA sequences are almost identical.

In this example, CARs that incorporated the variable regions of the anti-prostate mAb F77 55.10 were generated. T cells transduced with F77 CAR-encoding lentiviral vectors were found to be effective in in vitro functional tests and suppressed PC3 tumors in an in vivo tumor xenograft model. Both F77 CARs proved to be efficient at complete cytolysis of PC3 and RWPE-2 prostate cancer cells. However, F77 CAR1 harboring a 4-1BB ICD showed greater efficacy in both in vitro and in vivo functional assays. Both F77 CAR T cells induced a residual degree of cytolysis in prostate cancer cells RWPE-1 which correlates with the limited expression of F77 antigen on its cell surface as assessed by flow cytometry with mAb F77 55.10. Importantly, knockout of FUT1 gene drastically reduced mAb F77 binding to PC3 FUT1KO cells, and as consequence F77 CARs do not cause substantial cytolysis of PC3 FUT1KO target cells. The specificity of F77 CARs for target cells expressing the F77 antigen is further supported by the fact that no cytolysis of U87EGFRvIII target cells was seen. These F77 CAR T cells were also tested in a human RBC killing assay in comparison to mAb F77 55.10 where failure of F77 mediated killing of RBCs with F77 CAR T cells was observed while in case of mAb F77 55.10, killing was seen with blood types O and A but not blood type B at both 1% and 3% dilutions of RBCs used (Tables 5 and 6). This is consistent with the earlier findings, where that mAb F77 129 was found to exhibit binding capabilities toward analogs of blood groups A and B, albeit with a diminished binding affinity when compared to its interaction with blood group O. In contrast to F77 monoclonal antibodies, which demonstrate attributes akin to low-titer cold agglutinins, the F77 CAR T cells, intriguingly, do not partake in the F77-mediated killing of red blood cells (RBCs).

The present work describes the creation and validation of the first CAR that exclusively targets the F77 carbohydrate antigen expressed on prostate tumor cells.

TABLE 2
List of disclosed sequences and their corresponding SEQ ID NOs.

| | | | SEQ |
|---|---|---|---|
| | | | ID |
| Region | Description | Sequence | NO: |
| Variable | Mouse F77 heavy | QVQLKESGPGLVAPSQSLSITCTVSGFSLTYYGVH | 1 |
| Heavy | chain (clone ID: | WGRQSPGKGLEWLGIIWAGGNTNYNSTLKSRLSIS | |
| | 129.5), CDRs are | KDNSQSQVFLKMTSLQTDDTAMYYCARDDYAAM | |
| | underlined | DYWGQGTSVTVSS | |
| Variable | Mouse F77 Light | DVVMTQTPLSLPVSLGDQASISCRSSQTLVHSNGN | 2 |
| Light | chain (clone ID: | TFLHWYLKKPGQSPKLLIYKVSNRFSGVPDRFSGS | |
| | 129.5), CDRs are | GSGTHFTLKISRVEAEDLGVYFCSQGTHAPFTFGG | |
| | underlined | GTKLEIK | |
| Variable | Mouse F77 heavy | EVKLVESGPGLVAPSQSLSITCTVSGFSLTYYGVH | 3 |
| Heavy | chain (clone ID: | WGRQSPGKGLEWLGIIWAGGNTNYNSTLKSRLSIS | |
| | 129.16), CDRs | KDNSQSQVFLKMTSLQTDDTAMYYCARDDYAAM | |
| | are underlined | DYWGQGTS | |
| Variable | Mouse F77 light | ELVMTQSPLSLPVSLGDQASISCRSSQTLVHSNGNT | 4 |
| Light | chain (Clone ID: | FLHWYLKKPGQSPKLLIYKVSNRFSGVPDRESGSG | |
| | 129.16), CDRs | SGTHFTLKISRVEAEDLGVYFCSQGTHAPFTFGGG | |
| | are underlined | TKLEIKRA | |
| Variable | Mouse F77 heavy | QVQLKESGPGLVAPSQSLSITCTVSGFSLTYYGVH | 5 |
| Heavy | chain (clone ID: | WVRQPPGKGLEWLGIIWAGENTNYNSALMSRLSIS | |
| | 55.10), CDRs are | KDNSKSQVFLKVNSLQTDDTAIYYCARDDYAAMD | |
| | underlined | YWGQGTSVTVSS | |
| Variable | Mouse F77 Light | DVVMTQTPLSLPVSLGDQASISCRSSQSLVHSNGN | 6 |
| Light | chain (clone ID: | TFLHWYLQRPGQSPKLLIYKVSNRFSGVPDRESGS | |
| | 55.10), CDRs are | GSGTDFTLKISRVEAEDLGVYFCSQGSHVPFTFGG | |
| | underlined | GTKLEIK | |
| CDRs | CDR 1, heavy | GFSLTYYG | 7 |
| | chain | | |
| | CDR 1, heavy | GFTFTYYG | 8 |
| | chain | | |
| | CDR 2, heavy | IWAGGNT | 9 |
| | chain | | |
| | CDR 2, heavy | IWAGENT | 10 |
| | chain | | |
| | CDR 3, heavy | ARDDYAAMDY | 11 |
| | chain | | |
| | CDR 1, light | QTLVHSNGNTF | 12 |
| | chain | | |
| | CDR 1, light | QSLVHSNGNTF | 13 |
| | chain | | |
| | CDR 2, light | KVS | 14 |
| | chain | | |
| | CDR 3, light | SQGTHAPFT | 15 |
| | chain | | |
| | CDR 3, light | SQGSHVPFT | 16 |
| | chain | | |
| Variable | humanized F77 | QVQLQESGPGLVRPSQTLSLTCTVSGFTFTYYGVH | 17 |
| heavy | antibody clone | WVRQPPGRGLEWIGIIWAGGNTNYNSTLKSRVTM | |
| | 129.16, heavy | LVDTSKSQFSLRLSSVTAADTAVYYCARDDYAAM | |
| | chain, CDRs are | DYWGQGSLVTVSS | |
| | underlined | | |
| Variable | humanized F77 | DVVMTQSPSSLSVTLGQPASISCRSSQTLVHSNGNT | 18 |
| light | antibody clone | FLHWFQQRPGQSPRRLIYKVSNRFSGVPDRESGSG | |
| | 129.16, light | SGTDFTLTISRVEAEDVGVYYCSQGTHAPFTFGGG | |
| | chain, CDRs are | TKVEIK | |
| | underlined | | |
| Heavy | humanized F77 | QVQLQESGPGLVRPSQTLSLTCTVSGFTFTYYGVH | 19 |
| Chain | antibody clone | WVRQPPGRGLEWIGIIWAGGNTNYNSTLKSRVTM | |
| | 129.16, named | LVDTSKSQFSLRLSSVTAADTAVYYCARDDYAAM | |
| | F77N76S, | DYWGQGSLVTVSSAAATKGPSVFPLAPSSKSTSGG | |
| | heavy chain, | TAALGCLVKDYFPEPVTVSWNSGALTSGVHTFPAV | |
| | variable | LQSSGLYSLSSVVTVPSSSLGTQTYICNVNHKPSN | |
| | region is | TKVDKKVEPKSCDKTHTCPPCPAPELLGGPSVFLF | |
| | underlined | PPKPKDTLMISRTPEVTCVVVDVSHEDPEVKENWY | |
| | | VDGVEVHNAKTKPREEQYNSTYRVVSVLTVLHQD | |
| | | WLNGKEYKCKVSNKALPAPIEKTISKAKGQPREPQ | |
| | | VYTLPPSRDELTKNQVSLTCLVKGFYPSDIAVEWE | |
| | | SNGQPENNYKTTPPVLDSDGSFFLYSKLTVDKSRW | |
| | | QQGNVFSCSVMHEALHNHYTQKSLSLSPGK | |
| Heavy | humanized F77 | CAGGTCCAACTGCAGGAATCCGGTCCGGGTCTG | 20 |
| Chain | antibody clone | GTGCGTCCGTCGCAAACCCTGAGCCTGACCTGTA | |
| | 129.16, | CGGTTAGCGGCTTTACCTTCACGTATTACGGTGT | |
| | heavy chain, | GCACTGGGTTCGTCAGCCGCCGGGTCGTGGTCTG | |
| | nucleic acid | GAATGGATCGGTATTATCTGGGCGGGTGGCAAC | |
| | sequence | ACGAATTACAACTCTACCCTGAAAAGTCGTGTC | |
| | | ACGATGCTGGTTGATACCTCGAAAAGCCAATTC | |
| | | AGCCTGCGTCTGTCATCGGTGACCGCAGCAGAT | |
| | | ACGGCAGTTTATTACTGTGCTCGTGATGACTATG | |
| | | CAGCTATGGACTACTGGGGCCAGGGTAGCCTGG | |
| | | TTACCGTCAGCTCTGCGGCCGCAACCAAGGGCC | |
| | | CATCGGTCTTCCCCCTGGCACCCTCCTCCAAGAG | |
| | | CACCTCTGGGGGCACAGCAGCCCTGGGCTGCCT | |
| | | GGTCAAGGACTACTTCCCCGAACCGGTGACGGT | |
| | | GTCGTGGAACTCAGGCGCCCTGACCAGCGGCGT | |
| | | GCACACCTTCCCGGCTGTCCTACAGTCCTCAGGA | |
| | | CTCTACTCCCTCAGCAGCGTGGTGACCGTGCCCT | |
| | | CCAGCAGCTTGGGCACCCAGACCTACATCTGCA | |
| | | ACGTGAATCACAAGCCCAGCAACACCAAGGTGG | |
| | | ACAAGAAAGTTGAGCCCAAATCTTGTGACAAAA | |
| | | CTCACACATGCCCACCGTGCCCAGCACCTGAACT | |
| | | CCTGGGGGGACCGTCAGTCTTCCTCTTCCCCCCA | |
| | | AAACCCAAGGACACCCTCATGATCTCCCGGACC | |
| | | CCTGAGGTCACATGCGTGGTGGTGGACGTGAGC | |
| | | CACGAAGACCCTGAGGTCAAGTTCAACTGGTAC | |
| | | GTGGACGGCGTGGAGGTGCATAATGCCAAGACA | |
| | | AAGCCGCGGGAGGAGCAGTACAACAGCACGTAC | |
| | | CGTGTGGTCAGCGTCCTCACCGTCCTGCACCAGG | |
| | | ACTGGCTGAATGGCAAGGAGTACAAGTGCAAGG | |
| | | TCTCCAACAAAGCCCTCCCAGCCCCCATCGAGA | |
| | | AAACCATCTCCAAAGCCAAAGGGCAGCCCCGAG | |
| | | AACCACAGGTGTACACCCTGCCCCCATCCCGGG | |
| | | ATGAGCTGACCAAGAACCAGGTCAGCCTGACCT | |
| | | GCCTGGTCAAAGGCTTCTATCCCAGCGACATCGC | |
| | | CGTGGAGTGGGAGAGCAATGGGCAGCCGGAGA | |
| | | ACAACTACAAGACCACGCCTCCCGTGCTGGACT | |
| | | CCGACGGCTCCTTCTTCCTCTACAGCAAGCTCAC | |
| | | CGTGGACAAGAGCAGGTGGCAGCAGGGGAACG | |
| | | TCTTCTCATGCTCCGTGATGCATGAGGCTCTGCA | |
| | | CAACCACTACACACAGAAGAGCCTCTCCCTGTCT | |
| | | CCGGGTAAATGATTCTAGAGGGCCCGAACAAAA | |
| | | ACTCATCTCAGAAGAGGATCTGAATAGCGCCGT | |
| | | CGACCATCATCATCATCATCATGAGTTAGCA | |
| Light | humanized F77 | DVVMTQSPSSLSVTLGQPASISCRSSQTLVHSNGNT | 21 |
| Chain | antibody clone | FLHWFQQRPGQSPRRLIYKVSNRFSGVPDRESGSG | |
| | 129.16, variable | SGTDFTLTISRVEAEDVGVYYCSQGTHAPFTFGGG | |
| | region is | TKVEIKRRVATPSVFIFPPSDEQLKSGTASVVCLL | |
| | underlined | NNFYPREAKVQWKVDNALQSGNSQESVTEQDSKDS | |
| | | TYSLSSTLTLSKADYEKHKVYACEVTHQGLSSPVT | |
| | | KSFNRGEC | |
| Light | humanized F77 | GATGTCGTGATGACGCAGTCGCCTTCTAGTCTGA | 22 |
| Chain | antibody clone | GCGTGACCCTGGGACAGCCCGCCTCGATTTCGTG | |
| | 129.16, nucleic | TAGGAGTTCCCAGACGCTGGTGCACTCTAACGG | |
| | acid sequence | CAATACCTTCCTGCATTGGTTTCAGCAGAGGCCT | |
| | | GGACAGAGCCCACGCCGGCTGATCTACAAGGTG | |
| | | TCCAACCGATTCAGTGGCGTCCCTGACCGCTTTA | |
| | | GTGGATCTGGCTCGGGAACAGATTTCACCCTGA | |
| | | CAATTAGTAGAGTGGAGGCCGAAGACGTGGGCG | |
| | | TCTACTATTGCAGCCAGGGAACCCACGCTCCCTT | |
| | | CACATTTGGCGGAGGCACTAAGGTCGAGATCAA | |
| | | AAGGAGAGTGGCCACCCCAAGCGTCTTCATTTTT | |
| | | CCCCCATCCGACGAACAGCTGAAGTCTGGCACC | |
| | | GCCTCGGTGGTCTGTCTGCTGAACAACTTCTACC | |
| | | CAAGGGAGGCCAAGGTGCAGTGGAAAGTCGATA | |
| | | ACGCTCTGCAGAGCGGAAATTCCCAGGAGAGTG | |
| | | TGACAGAACAGGACTCTAAGGATTCGACTTATA | |
| | | GCCTGAGCTCCACTCTGACGCTGAGCAAAGCCG | |
| | | ATTACGAGAAGCATAAAGTGTATGCTTGCGAAG | |
| | | TGACGCACCAGGGACTGAGTTCGCCCGTGACAA | |
| | | AATCGTTCAACAGAGGAGAATGC | |
| Variable | Consensus H3, | EVKLVESGGGLVQPGGSLRLSCAASGF.... | 23 |
| Heavy | double mutant | WVRQAPGKGLEWVGAI..... | |
| | Q75K/T83N, | KGRFTISRDNSKNTLYLQMNSLRAEDTAVYYCAR. | |
| | framework | ..FDYWGQGTL | |
| | regions | | |
| Variable | Consensus H3, | EVQLVESGGGLVQPGGSLRLSCAASGF.... | 24 |
| Heavy | triple mutant | WVRQAPGKGLEWVGAI..... | |
| | K3Q/Q75K/T83N, | KGRFTISRDNSKNTLYLQMNSLRAEDTAVYYCAR. | |
| | framework | ..FDYWGQGTL | |
| | regions | | |
| Linker | 20 amino acids | GGGGSGGGGSGGGGSGGGGS | 25 |
| Linker | 30 amino acids | GGGGSGGGGSGGGGSGGGGSGGGGSGGGGS | 26 |
| scFv | F77 scFv | EVKLVESGPGLVAPSQSLSITCTVSGFSLTYYGVH | 27 |
| | fragment, amino | WGRQSPGKGLEWLGIIWAGGNTNYNSTLKSRLSIS | |
| | acid sequence, | KDNSQSQVFLKMTSLQTDDTAMYYCARDDYAAMD | |
| | linker region | YWGQGTSGGGGSGGGGSGGGGSGGGGSSMADVVM | |
| | underlined | TQSPSSLSVTLGQPASISCRSSQTLVHSNGNTFL | |
| | | HWFQQRPGQSPRRLIYKVSNRFSGVPDRFSGSGSG | |
| | | TDFTLTISRVEAEDVGVYYCSQGTHAPFTFGGGTK | |
| | | VEIKRR | |
| scFab | F77 scFab | EVKLVESGPGLVAPSQSLSITCTVSGFSLTYYGVH | 28 |
| | fragment, amino | WGRQSPGKGLEWLGIIWAGGNTNYNSTLKSRLSIS | |
| | acid sequence, | KDNSQSQVFLKMTSLQTDDTAMYYCARDDYAAMD | |
| | linker region | YWGQGSLVTVSSAAATKGPSVFPLAPSSKSTSGG | |
| | underlined | TAALGCLVKDYFPEPVTVSWNSGALTSGVHTFPA | |
| | | VLQSSGLYSLSSVVTVPSSSLGTQTYICNVNHKPS | |
| | | NTKVDKKVEPKSGGGGSGGGGSGGGGSGGGGSGG | |
| | | GGSGGGGSSMADVVMTQSPSSLSVTLGQPASISCR | |
| | | SSQTLVHSNGNTFLHWFQQRPGQSPRRLIYKVSNR | |
| | | FSGVPDRFSGSGSGTDFTLTISRVEAEDVGVYYCSQ | |
| | | GTHAPFTFGGGTKVEIKRRVATPSVFIFPPSDEQLK | |
| | | SGTASVVCLLNNFYPREAKVQWKVDNALQSGNSQ | |
| | | ESVTEQDSKDSTYSLSSTLTLSKADYEKHKVYACE | |
| | | VTHQGLSSPVTKSFNRGEC | |
| Variable | Mouse F77 heavy | EVKLVESGPGLVAPSQSLSITCTVSGFSLTYYGVH | 29 |
| heavy | chain (clone ID: | WGRQSPGKGLEWLGIIWAGGNTNYNSTLKSRLSIS | |
| | 129.16), mF77H, | KDNSQSQVFLKMTSLQTDDTAMYYCARDDYAAM | |
| | CDRs are | DYWGQGT | |
| | underlined | | |
| Variable | Mouse F77 heavy | ELVMTQSPLSLPVSLGDQASISCRSSQTLVHSNGNT | 30 |
| Light | chain (clone ID: | FLHWYLKKPGQSPKLLIYKVSNRFSGVPDRESGSG | |
| | 129.16), mF77k, | SGTHFTLKISRVEAEDLGVYFCSQGTHAPFTFGGG | |
| | CDRs are | TKLEIK | |
| | underlined | | |
| Variable | Human germline | QVQLQESGPGLVKPSETLSLTCTVSGGSISSYYWS | 31 |
| heavy | sequence, | WIRQPPGKGLEWIGYIYTSGSTNYNPSLKSRVTIS | |
| | hIGHV4-4*08 | VDTSKNQFSLKLSSVTAADTAVYYCAR---- | |
| | | YFDYWGQGSLVTVSS | |
| Variable | Human germline | DVVMTQSPLSLPVTLGQPASISCRSSQSLVHSDGNT | 32 |
| light | sequence, | YLNWFQQRPGQSPRRLIYKVSNRDSGVPDRFSGSG | |
| | hIGKV2-30*02 | SGTDFTLKISRVEAEDVGVYYCMQGTHWPCTFGQ | |
| | | GTKLEIK | |
| Variable | Mouse F77 heavy | EVQLVESGPGLVAPSQSLSITCTVSGFSLTYYGVH | 33 |
| heavy | chain (clone ID: | WGRQSPGKGLEWLGIIWAGGNTNYNSTLKSRLSIS | |
| | 129.16), triple | KDNSKSQVFLKMNSLQTDDTAMYYCARDDYAAM | |
| | mutant, | DYWGQGTS | |
| | mF77Hmutant, | | |
| | CDRs are | | |
| | underlined | | |
| F77 | Nucleic acid | CAAGTGCAGCTGAAAGAAAGCGGACCCGGT | 34 |
| VHVL | sequence | CTGGTGGCCCCCAGCCAGTCTTTAAGCATTA | |
| 41BBzeta | | CTTGTACCGTGAGCGGCTTTTCTTTAACCTA | |
| | | CTACGGCGTCCACTGGGTGAGACAGCCTCCC | |
| | | GGCAAGGGACTGGAGTGGCTGGGCATCATC | |
| | | TGGGCCGGCGAGAACACCAACTACAACAGC | |
| | | GCTTTAATGTCTCGTCTGAGCATCAGCAAGG | |
| | | ACAACAGCAAGAGCCAAGTTTTTTTAAAGGT | |
| | | GAACTCTTTACAGACCGACGACACCGCTATC | |
| | | TACTACTGCGCTCGTGACGACTACGCCGCCA | |
| | | TGGACTACTGGGGCCAAGGTACAAGCGTGA | |
| | | CAGTGTCCGGCGGAGGCGGAAGCGGCGGCG | |
| | | GCGGCAGCAGCGGCGGAGGCAGCGACGTGG | |
| | | TGATGACCCAGACACCTTTAAGCCTCCCCGT | |
| | | TTCTTTAGGCGATCAAGCTTCCATCAGCTGT | |
| | | CGTAGCAGCCAATCTTTAGTGCACAGCAATG | |
| | | GCAACACCTTTTTACACTGGTATTTACAGAG | |
| | | ACCCGGACAGAGCCCCAAGCTGCTGATCTA | |
| | | CAAGGTGTCCAATCGTTTCAGCGGCGTCCCC | |
| | | GATCGTTTTAGCGGCAGCGGCAGCGGAACC | |
| | | GATTTCACTTTAAAGATTTCTCGTGTGGAGG | |
| | | CCGAGGACCTCGGCGTGTACTTCTGTAGCCA | |
| | | AGGTAGCCACGTGCCCTTCACATTCGGCGGC | |
| | | GGCACCAAGCTGGAGATCAAGAGAGCCGCT | |
| | | AGCACCACTACCCCAGCACCGAGGCCACCC | |
| | | ACCCCGGCTCCTACCATCGCCTCCCAGCCTC | |
| | | TGTCCCTGCGTCCGGAGGCATGTAGACCCGC | |
| | | AGCTGGTGGGGCCGTGCATACCCGGGGTCTT | |
| | | GACTTCGCCTGCGATATCTACATTTGGGCCC | |
| | | CTCTGGCTGGTACTTGCGGGGTCCTGCTGCT | |
| | | TTCACTCGTGATCACTCTTTACTGTAAGCGC | |
| | | GGTCGGAAGAAGCTGCTGTACATCTTTAAGC | |
| | | AACCCTTCATGAGGCCTGTGCAGACTACTCA | |
| | | AGAGGAGGACGGCTGTTCATGCCGGTTCCC | |
| | | AGAGGAGGAGGAAGGCGGCTGCGAACTGCG | |
| | | CGTGAAATTCAGCCGCAGCGCAGATGCTCC | |
| | | AGCCTACAAGCAGGGGCAGAACCAGCTCTA | |
| | | CAACGAACTCAATCTTGGTCGGAGAGAGGA | |
| | | GTACGACGTGCTGGACAAGCGGAGAGGACG | |
| | | GGACCCAGAAATGGGCGGGAAGCCGCGCAG | |
| | | AAAGAATCCCCAAGAGGGCCTGTACAACGA | |
| | | GCTCCAAAAGGATAAGATGGCAGAAGCCTA | |
| | | TAGCGAGATTGGTATGAAAGGGGAACGCAG | |
| | | AAGAGGCAAAGGCCACGACGGACTGTACCA | |
| | | GGGACTCAGCACCGCCACCAAGGACACCTA | |
| | | TGACGCTCTTCACATGCAGGCCCTGCCGCCT | |
| | | CGGTGA | |
| F77 | Nucleic acid | GACGTGGTGATGACCCAGACCCCTCTGAGCC | 35 |
| VLVH | sequence | TGCCTGTGAGCCTGGGCGACCAGGCCAGCA | |
| 41BBzeta | | TCAGCTGCAGGAGCAGCCAGAGCCTGGTGC | |
| | | ACAGCAACGGCAACACCTTCCTGCACTGGTA | |
| | | CCTGCAGAGGCCTGGCCAGAGCCCTAAGCT | |
| | | GCTGATCTACAAGGTGAGCAACAGGTTCAG | |
| | | CGGCGTGCCTGACAGGTTCAGCGGCAGCGG | |
| | | CAGCGGCACCGACTTCACCCTGAAGATCAG | |
| | | CAGGGTGGAGGCCGAGGACCTGGGCGTGTA | |
| | | CTTCTGCAGCCAGGGCAGCCACGTGCCTTTC | |
| | | ACCTTCGGCGGCGGCACCAAGCTGGAGATC | |
| | | AAGAGGGCCGGCGGCGGCGGCAGCGGCGGC | |
| | | GGCGGCAGCAGCGGCGGCGGCAGCCAGGTG | |
| | | CAGCTGAAGGAGAGCGGCCCTGGCCTGGTG | |
| | | GCCCCTAGCCAGAGCCTGAGCATCACCTGCA | |
| | | CCGTGAGCGGCTTCAGCCTGACCTACTACGG | |
| | | CGTGCACTGGGTGAGGCAGCCTCCTGGCAA | |
| | | GGGCCTGGAGTGGCTGGGCATCATCTGGGC | |
| | | CGGCGAGAACACCAACTACAACAGCGCCCT | |
| | | GATGAGCAGGCTGAGCATCAGCAAGGACAA | |
| | | CAGCAAGAGCCAGGTGTTCCTGAAGGTGAA | |
| | | CAGCCTGCAGACCGACGACACCGCCATCTA | |
| | | CTACTGCGCCAGGGACGACTACGCCGCCAT | |
| | | GGACTACTGGGGCCAGGGCACCAGCGTGAC | |
| | | CGTGAGCGCTAGCACCACTACCCCAGCACC | |
| | | GAGGCCACCCACCCCGGCTCCTACCATCGCC | |
| | | TCCCAGCCTCTGTCCCTGCGTCCGGAGGCAT | |
| | | GTAGACCCGCAGCTGGTGGGGCCGTGCATA | |
| | | CCCGGGGTCTTGACTTCGCCTGCGATATCTA | |
| | | CATTTGGGCCCCTCTGGCTGGTACTTGCGGG | |
| | | GTCCTGCTGCTTTCACTCGTGATCACTCTTTA | |
| | | CTGTAAGCGCGGTCGGAAGAAGCTGCTGTA | |
| | | CATCTTTAAGCAACCCTTCATGAGGCCTGTG | |
| | | CAGACTACTCAAGAGGAGGACGGCTGTTCA | |
| | | TGCCGGTTCCCAGAGGAGGAGGAAGGCGGC | |
| | | TGCGAACTGCGCGTGAAATTCAGCCGCAGC | |
| | | GCAGATGCTCCAGCCTACAAGCAGGGGCAG | |
| | | AACCAGCTCTACAACGAACTCAATCTTGGTC | |
| | | GGAGAGAGGAGTACGACGTGCTGGACAAGC | |
| | | GGAGAGGACGGGACCCAGAAATGGGCGGG | |
| | | AAGCCGCGCAGAAAGAATCCCCAAGAGGGC | |
| | | CTGTACAACGAGCTCCAAAAGGATAAGATG | |
| | | GCAGAAGCCTATAGCGAGATTGGTATGAAA | |
| | | GGGGAACGCAGAAGAGGCAAAGGCCACGA | |
| | | CGGACTGTACCAGGGACTCAGCACCGCCAC | |
| | | CAAGGACACCTATGACGCTCTTCACATGCAG | |
| | | GCCCTGCCGCCTCGGTGA | |
| F77 | Nucleic acid | CAAGTGCAGCTGAAAGAAAGCGGACCCGGT | 36 |
| VHVL | sequence | CTGGTGGCCCCCAGCCAGTCTTTAAGCATTA | |
| CD28zeta | | CTTGTACCGTGAGCGGCTTTTCTTTAACCTA | |
| | | CTACGGCGTCCACTGGGTGAGACAGCCTCCC | |
| | | GGCAAGGGACTGGAGTGGCTGGGCATCATC | |
| | | TGGGCCGGCGAGAACACCAACTACAACAGC | |
| | | GCTTTAATGTCTCGTCTGAGCATCAGCAAGG | |
| | | ACAACAGCAAGAGCCAAGTTTTTTTAAAGGT | |
| | | GAACTCTTTACAGACCGACGACACCGCTATC | |
| | | TACTACTGCGCTCGTGACGACTACGCCGCCA | |
| | | TGGACTACTGGGGCCAAGGTACAAGCGTGA | |
| | | CAGTGTCCGGCGGAGGCGGAAGCGGCGGCG | |
| | | GCGGCAGCAGCGGCGGAGGCAGCGACGTGG | |
| | | TGATGACCCAGACACCTTTAAGCCTCCCCGT | |
| | | TTCTTTAGGCGATCAAGCTTCCATCAGCTGT | |
| | | CGTAGCAGCCAATCTTTAGTGCACAGCAATG | |
| | | GCAACACCTTTTTACACTGGTATTTACAGAG | |
| | | ACCCGGACAGAGCCCCAAGCTGCTGATCTA | |
| | | CAAGGTGTCCAATCGTTTCAGCGGCGTCCCC | |
| | | GATCGTTTTAGCGGCAGCGGCAGCGGAACC | |
| | | GATTTCACTTTAAAGATTTCTCGTGTGGAGG | |
| | | CCGAGGACCTCGGCGTGTACTTCTGTAGCCA | |
| | | AGGTAGCCACGTGCCCTTCACATTCGGCGGC | |
| | | GGCACCAAGCTGGAGATCAAGAGAGCCGCT | |
| | | AGCACCACGACGCCAGCGCCGCGACCACCA | |
| | | ACACCGGCGCCCACCATCGCGTCGCAGCCC | |
| | | CTGTCCCTGCGCCCAGAGGCGTGCCGGCCAG | |
| | | CGGCGGGGGGCGCAGTGCACACGAGGGGGC | |
| | | TGGACTTCGCCTGTGATTTTTGGGTGCTGGT | |
| | | GGTGGTTGGTGGAGTCCTGGCTTGCTATAGC | |
| | | TTGCTAGTAACAGTGGCCTTTATTATTTTCTG | |
| | | GGTGAGGAGTAAGAGGAGCAGGCTCCTGCA | |
| | | CAGTGACTACATGAACATGACTCCCCGCCGC | |
| | | CCCGGGCCCACCCGCAAGCATTACCAGCCCT | |
| | | ATGCCCCACCACGCGACTTCGCAGCCTATCG | |
| | | CTCCCTGAGAGTGAAGTTCAGCAGGAGCGC | |
| | | AGACGCCCCCGCGTACCAGCAGGGCCAGAA | |
| | | CCAGCTCTATAACGAGCTCAATCTAGGACGA | |
| | | AGAGAGGAGTACGATGTTTTGGACAAGAGA | |
| | | CGTGGCCGGGACCCTGAGATGGGGGGAAAG | |
| | | CCGAGAAGGAAGAACCCTCAGGAAGGCCTG | |
| | | TACAATGAACTGCAGAAAGATAAGATGGCG | |
| | | GAGGCCTACAGTGAGATTGGGATGAAAGGC | |
| | | GAGCGCCGGAGGGGCAAGGGGCACGATGGC | |
| | | CTTTACCAGGGTCTCAGTACAGCCACCAAGG | |
| | | ACACCTACGACGCCCTTCACATGCAGGCCCT | |
| | | GCCCCCTCGC | |
| F77 | Nucleic acid | GACGTGGTGATGACCCAGACCCCTCTGAGCC | 37 |
| VLVH | sequence | TGCCTGTGAGCCTGGGCGACCAGGCCAGCA | |
| CD28zeta | | TCAGCTGCAGGAGCAGCCAGAGCCTGGTGC | |
| | | ACAGCAACGGCAACACCTTCCTGCACTGGTA | |
| | | CCTGCAGAGGCCTGGCCAGAGCCCTAAGCT | |
| | | GCTGATCTACAAGGTGAGCAACAGGTTCAG | |
| | | CGGCGTGCCTGACAGGTTCAGCGGCAGCGG | |
| | | CAGCGGCACCGACTTCACCCTGAAGATCAG | |
| | | CAGGGTGGAGGCCGAGGACCTGGGCGTGTA | |
| | | CTTCTGCAGCCAGGGCAGCCACGTGCCTTTC | |
| | | ACCTTCGGCGGCGGCACCAAGCTGGAGATC | |
| | | AAGAGGGCCGGCGGCGGCGGCAGCGGCGGC | |
| | | GGCGGCAGCAGCGGCGGCGGCAGCCAGGTG | |
| | | CAGCTGAAGGAGAGCGGCCCTGGCCTGGTG | |
| | | GCCCCTAGCCAGAGCCTGAGCATCACCTGCA | |
| | | CCGTGAGCGGCTTCAGCCTGACCTACTACGG | |
| | | CGTGCACTGGGTGAGGCAGCCTCCTGGCAA | |
| | | GGGCCTGGAGTGGCTGGGCATCATCTGGGC | |
| | | CGGCGAGAACACCAACTACAACAGCGCCCT | |
| | | GATGAGCAGGCTGAGCATCAGCAAGGACAA | |
| | | CAGCAAGAGCCAGGTGTTCCTGAAGGTGAA | |
| | | CAGCCTGCAGACCGACGACACCGCCATCTA | |
| | | CTACTGCGCCAGGGACGACTACGCCGCCAT | |
| | | GGACTACTGGGGCCAGGGCACCAGCGTGAC | |
| | | CGTGAGCGCTAGCACCACGACGCCAGCGCC | |
| | | GCGACCACCAACACCGGCGCCCACCATCGC | |
| | | GTCGCAGCCCCTGTCCCTGCGCCCAGAGGCG | |
| | | TGCCGGCCAGCGGCGGGGGGCGCAGTGCAC | |
| | | ACGAGGGGGCTGGACTTCGCCTGTGATTTTT | |
| | | GGGTGCTGGTGGTGGTTGGTGGAGTCCTGGC | |
| | | TTGCTATAGCTTGCTAGTAACAGTGGCCTTT | |
| | | ATTATTTTCTGGGTGAGGAGTAAGAGGAGC | |
| | | AGGCTCCTGCACAGTGACTACATGAACATG | |
| | | ACTCCCCGCCGCCCCGGGCCCACCCGCAAG | |
| | | CATTACCAGCCCTATGCCCCACCACGCGACT | |
| | | TCGCAGCCTATCGCTCCCTGAGAGTGAAGTT | |
| | | CAGCAGGAGCGCAGACGCCCCCGCGTACCA | |
| | | GCAGGGCCAGAACCAGCTCTATAACGAGCT | |
| | | CAATCTAGGACGAAGAGAGGAGTACGATGT | |
| | | TTTGGACAAGAGACGTGGCCGGGACCCTGA | |
| | | GATGGGGGGAAAGCCGAGAAGGAAGAACC | |
| | | CTCAGGAAGGCCTGTACAATGAACTGCAGA | |
| | | AAGATAAGATGGCGGAGGCCTACAGTGAGA | |
| | | TTGGGATGAAAGGCGAGCGCCGGAGGGGCA | |
| | | AGGGGCACGATGGCCTTTACCAGGGTCTCA | |
| | | GTACAGCCACCAAGGACACCTACGACGCCC | |
| | | TTCACATGCAGGCCCTGCCCCCTCGC | |
| F77 | Amino acid | QVQLKESGPGLVAPSQSLSITCTVSGFSLTYYG | 38 |
| VHVL | sequence | VHWVRQPPGKGLEWLGIIWAGENTNYNSALM | |
| 41BBzeta | | SRLSISKDNSKSQVFLKVNSLQTDDTAIYYCAR | |
| | | DDYAAMDYWGQGTSVTVSGGGGSGGGGSSG | |
| | | GGSDVVMTQTPLSLPVSLGDQASISCRSSQSLV | |
| | | HSNGNTFLHWYLQRPGQSPKLLIYKVSNRFSG | |
| | | VPDRFSGSGSGTDFTLKISRVEAEDLGVYFCSQ | |
| | | GSHVPFTFGGGTKLEIKRATTTPAPRPPTPAPTI | |
| | | ASQPLSLRPEACRPAAGGAVHTRGLDFACDIYI | |
| | | WAPLAGTCGVLLLSLVITLYCKRGRKKLLYIF | |
| | | KQPFMRPVQTTQEEDGCSCRFPEEEEGGCELR | |
| | | VKFSRSADAPAYKQGQNQLYNELNLGRREEY | |
| | | DVLDKRRGRDPEMGGKPRRKNPQEGLYNELQ | |
| | | KDKMAEAYSEIGMKGERRRGKGHDGLYQGL | |
| | | STATKDTYDALHMQALPPR | |
| F77 | Amino acid | DVVMTQTPLSLPVSLGDQASISCRSSQSLVHSN | 39 |
| VLVH | sequence | GNTFLHWYLQRPGQSPKLLIYKVSNRFSGVPD | |
| 41BBzeta | | RFSGSGSGTDFTLKISRVEAEDLGVYFCSQGSH | |
| | | VPFTFGGGTKLEIKRAGGGGSGGGGSSGGGSQ | |
| | | VQLKESGPGLVAPSQSLSITCTVSGFSLTYYGV | |
| | | HWVRQPPGKGLEWLGIIWAGENTNYNSALMS | |
| | | RLSISKDNSKSQVFLKVNSLQTDDTAIYYCAR | |
| | | DDYAAMDYWGQGTSVTVSASTTTPAPRPPTP | |
| | | APTIASQPLSLRPEACRPAAGGAVHTRGLDFA | |
| | | CDIYIWAPLAGTCGVLLLSLVITLYCKRGRKK | |
| | | LLYIFKQPFMRPVQTTQEEDGCSCRFPEEEEGG | |
| | | CELRVKFSRSADAPAYKQGQNQLYNELNLGR | |
| | | REEYDVLDKRRGRDPEMGGKPRRKNPQEGLY | |
| | | NELQKDKMAEAYSEIGMKGERRRGKGHDGL | |
| | | YQGLSTATKDTYDALHMQALPPR | |
| F77 | Amino acid | QVQLKESGPGLVAPSQSLSITCTVSGFSLTYYG | 40 |
| VHVL | sequence | VHWVRQPPGKGLEWLGIIWAGENTNYNSALM | |
| CD28zeta | | SRLSISKDNSKSQVFLKVNSLQTDDTAIYYCAR | |
| | | DDYAAMDYWGQGTSVTVSGGGGSGGGGSSG | |
| | | GGSDVVMTQTPLSLPVSLGDQASISCRSSQSLV | |
| | | HSNGNTFLHWYLQRPGQSPKLLIYKVSNRFSG | |
| | | VPDRFSGSGSGTDFTLKISRVEAEDLGVYFCSQ | |
| | | GSHVPFTFGGGTKLEIKRATTTPAPRPPTPAPTI | |
| | | ASQPLSLRPEACRPAAGGAVHTRGLDFACDF | |
| | | WVLVVVGGVLACYSLLVTVAFIIFWVRSKRSR | |
| | | LLHSDYMNMTPRRPGPTRKHYQPYAPPRDFA | |
| | | AYRSRVKFSRSADAPAYQQGQNQLYNELNLG | |
| | | RREEYDVLDKRRGRDPEMGGKPRRKNPQEGL | |
| | | YNELQKDKMAEAYSEIGMKGERRRGKGHDG | |
| | | LYQGLSTATKDTYDALHMQALPPR | |
| F77 | Amino acid | DVVMTQTPLSLPVSLGDQASISCRSSQSLVHSN | 41 |
| VLVH | sequence | GNTFLHWYLQRPGQSPKLLIYKVSNRFSGVPD | |
| CD28zeta | | RFSGSGSGTDFTLKISRVEAEDLGVYFCSQGSH | |
| | | VPFTFGGGTKLEIKRAGGGGSGGGGSSGGGSQ | |
| | | VQLKESGPGLVAPSQSLSITCTVSGFSLTYYGV | |
| | | HWVRQPPGKGLEWLGIIWAGENTNYNSALMS | |
| | | RLSISKDNSKSQVFLKVNSLQTDDTAIYYCAR | |
| | | DDYAAMDYWGQGTSVTVSTTTPAPRPPTPAP | |
| | | TIASQPLSLRPEACRPAAGGAVHTRGLDFACD | |
| | | FWVLVVVGGVLACYSLLVTVAFIIFWVRSKRS | |
| | | RLLHSDYMNMTPRRPGPTRKHYQPYAPPRDF | |
| | | AAYRSRVKFSRSADAPAYQQGQNQLYNELNL | |
| | | GRREEYDVLDKRRGRDPEMGGKPRRKNPQEG | |
| | | LYNELQKDKMAEAYSEIGMKGERRRGKGHD | |
| | | GLYQGLSTATKDTYDALHMQALPPR | |
| 41BBzeta | Amino acid | IYIWAPLAGTCGVLLLSLVITLYCKRGRKKLLY | 42 |
| | sequence | IFKQPFMRPVQTTQEEDGCSCRFPEEEEGGCEL | |
| | | RVKFSRSADAPAYKQGQNQLYNELNLGRREE | |
| | | YDVLDKRRGRDPEMGGKPRRKNPQEGLYNEL | |
| | | QKDKMAEAYSEIGMKGERRRGKGHDGLYQG | |
| | | LSTATKDTYDALHMQALPPR | |
| CD28zeta | Amino acid | FWVLVVVGGVLACYSLLVTVAFIIFWVRSKRS | 43 |
| | sequence | RLLHSDYMNMTPRRPGPTRKHYQPYAPPRDF | |
| | | AAYRSRVKFSRSADAPAYQQGQNQLYNELNL | |
| | | GRREEYDVLDKRRGRDPEMGGKPRRKNPQEG | |
| | | LYNELQKDKMAEAYSEIGMKGERRRGKGHD | |
| | | GLYQGLSTATKDTYDALHMQALPPR | |
| Linker | Amino acid | GGGGSGGGGSSGGGS | 44 |
| | sequence | | |

TABLE 3
Amino acid sequences of the F77 CAR domains.

| | | SEQ |
|---|---|---|
| Domain | | ID |
| Name | Amino Acid Sequence | NO: |
| F77 VH | QVQLKESGPGLVAPSQSLSITCTVSGFSLTYYG | 45 |
| | VHWVRQPPGKGLEWLGIIWAGENTNYNSALMSR | |
| | LSISKDNSKSQVFLKVNSLQTDDTAIYYCAR | |
| | DDYAAMDYWGQGTSVTVS | |
| F77 VL | DVVMTQTPLSLPVSLGDQASISCRSSQSLVHSN | 46 |
| | GNTFLHWYLQRPGQSPKLLIYKVSNRFSGVPDR | |
| | FSGSGSGTDFTLKISRVEAEDLGVYFCSQGSH | |
| | VPFTFGGGTKLEIKRA | |
| Linker | GGGGSGGGGSSGGGS | 44 |
| CD8 | TTTPAPRPPTPAPTIASQPLSLRPEACRPAAGG | 47 |
| Hinge | AVHTRGLDFACD | |
| CD8 TM | IYIWAPLAGTCGVLLLSLVITLYC | 48 |
| 4-1BB | KRGRKKLLYIFKQPFMRPVQTTQEEDGCSCRF | 49 |
| ICD | PEEEEGGCEL | |
| CD28 TM | FWVLVVVGGVLACYSLLVTVAFIIFWV | 50 |
| CD28 | RSKRSRLLHSDYMNMTPRRPGPTRKHYQPYA | 51 |
| ICD | PPRDFAAYRS | |
| CD3 zeta | RVKFSRSADAPAYKQGQNQLYNELNLGRREE | 52 |
| (with 4- | YDVLDKRRGRDPEMGGKPRRKNPQEGLYNEL | |
| 1BB) | QKDKMAEAYSEIGMKGERRRGKGHDGLYQG | |
| | LSTATKDTYDALHMQALPPR | |
| CD3 zeta | RVKFSRSADAPAYQQGQNQLYNELNLGRREE | 53 |
| (with | YDVLDKRRGRDPEMGGKPRRKNPQEGLYNEL | |
| CD28) | QKDKMAEAYSEIGMKGERRRGKGHDGLYQG | |
| | LSTATKDTYDALHMQALPPR | |

### Illustrative Embodiments

Provided here are illustrative embodiments of the disclosed technology. These embodiments are illustrative only and do not limit the scope of the present disclosure or of the claims attached.

Embodiment 1. A single chain variable fragment (scFv), comprising a heavy chain variable region, a linker region, and a light chain variable region, wherein:
the heavy chain variable region comprises heavy chain complementarity determining region 1 (CDR1), CDR2, and CDR3 of SEQ ID NOs: 8, 9, and 11, and the light chain variable region comprises light chain CDR1, CDR2, and CDR3 of SEQ ID NOs: 12, 14, and 15;
the heavy chain variable region comprises heavy chain CDR1, CDR2, and CDR3 of SEQ ID NOs: 7, 9, and 11, and the light chain variable region comprises light chain CDR1, CDR2, and CDR3 of SEQ ID NOs: 12, 14, and 15; or
the heavy chain variable region comprises heavy chain CDR1, CDR2, and CDR3 of SEQ ID NOs: 7, 10, and 11, and the light chain variable region comprises light chain CDR1, CDR2, and CDR3 of SEQ ID NOs: 13, 14, and 16.

Embodiment 2. The scFv of embodiment 1, wherein the heavy chain variable region comprises SEQ ID NO: 1 and the light chain variable region comprises SEQ ID NO: 2.

Embodiment 3. The scFv of embodiment 1, wherein the heavy chain variable region comprises SEQ ID NO: 3 and the light chain variable region comprises SEQ ID NO: 4.

Embodiment 4. The scFv of embodiment 1, wherein the heavy chain variable region comprises SEQ ID NO: 5 and the light chain variable region comprises SEQ ID NO: 6.

Embodiment 5. The scFv of any one of the previous embodiments, wherein the heavy chain variable region, the light chain variable region, or both the heavy chain variable region and the light chain variable region is humanized.

Embodiment 6. The scFv of embodiment 5, wherein the heavy chain variable region comprises SEQ ID NO: 17 and the light chain variable region comprises SEQ ID NO: 18.

Embodiment 7. The scFv of embodiment 5, wherein framework regions of the heavy chain variable region have about 95% sequence identity to the framework regions of SEQ ID NO: 23 or SEQ ID NO: 24.

Embodiment 8. The scFv of any one of the previous embodiments, wherein the linker region comprises SEQ ID NO: 25 or SEQ ID NO: 26.

Embodiment 9. The scFv of any one of the previous embodiments fused to a transmembrane domain.

Embodiment 10. The scFv of any one of the previous embodiments, fused to a transmembrane domain and a signal transduction domain.

Embodiment 11. The scFv of any one of the previous embodiments, fused to transmembrane domain and an immune-costimulatory domain.

Embodiment 12. The scFv of embodiment 11, wherein the co-stimulatory domain comprises the amino acid sequence of SEQ ID NO: 42.

Embodiment 13. The scFv of embodiment 11, wherein the co-stimulatory domain comprises the amino acid sequence of SEQ ID NO: 43.

Embodiment 14. A single chain antigen binding fragment (scFab) comprising a heavy chain region, a linker region, and a light chain region, the light chain region comprising a light chain variable region and a light chain constant region, wherein
the heavy chain region comprises heavy chain CDR1, CDR2, and CDR3 of SEQ ID NOs: 8, 9, and 11, and the light chain variable region comprises light chain CDR1, CDR2, and CDR3 of SEQ ID NOs: 12, 14, and 15;
the heavy chain region comprises heavy chain CDR1, CDR2, and CDR3 of SEQ ID NOs: 7, 9, and 11, and the light chain variable region comprises light chain CDR1, CDR2, and CDR3 of SEQ ID NOs: 12, 14, and 15; or
the heavy chain region comprises heavy chain CDR1, CDR2, and CDR3 of SEQ ID NOs: 7, 10, and 11, and the light chain variable region comprises light chain CDR1, CDR2, and CDR3 of SEQ ID NOs: 13, 14, and 16.

Embodiment 15. The scFab of embodiment 14, wherein the heavy chain region comprises SEQ ID NO: 1 and the light chain region comprises SEQ ID NO: 2.

Embodiment 16. The scFab of embodiment 14, wherein the heavy chain region comprises SEQ ID NO: 3 and the light chain region comprises SEQ ID NO: 4.

Embodiment 17. The scFab of embodiment 14, wherein the heavy chain region comprises SEQ ID NO: 5 and the light chain region comprises SEQ ID NO: 6.

Embodiment 18. The scFab of any one of embodiments 14-17, wherein the heavy chain region, the light chain region, or both the heavy chain region and the light chain region is humanized.

Embodiment 19. The scFab of embodiment 18, wherein the heavy chain region comprises SEQ ID NO: 17 and the light chain region comprises SEQ ID NO: 18.

Embodiment 20. The scFab of embodiment 18, wherein framework regions of the heavy chain region have about 95% sequence identity to the framework regions of SEQ ID NO: 23 or SEQ ID NO: 24.

Embodiment 21. The scFab of any one of embodiments 14-20, wherein the linker region comprises SEQ ID NO: 25 or SEQ ID NO: 26.

Embodiment 22. An antibody comprising a heavy chain having an amino acid sequence according to SEQ ID NO: 19 and a light chain having an amino acid sequence according to SEQ ID NO: 21.

Embodiment 23. A composition comprising the scFv of any one of embodiments 1-13, the scFab of any one of embodiments 14-21, or the antibody of embodiment 22.

Embodiment 24. The composition of embodiment 23 comprising the scFv of any one of embodiments 5-13 or the scFab of any one of embodiments 18-21.

Embodiment 25. A chimeric antigen receptor (CAR) comprising the scFv of any one of embodiments 1-13.

Embodiment 26. The CAR of embodiment 25 comprising a CD8 transmembrane domain (CD8 TM), 4-1BB intracellular domain (4-1BB ICD), and a CD3 zeta intracellular signaling domain.

Embodiment 27. The CAR of embodiment 25 or 26 comprising CD8 TM of SEQ ID NO: 48, 4-1BB ICD of SEQ ID NO: 49, and a CD3 zeta intracellular signaling domain of SEQ ID NO: 52.

Embodiment 28. The CAR of any one of embodiments 25-27, comprising the amino acid sequence of SEQ ID NO: 38 or 39.

Embodiment 29. The CAR of embodiment 25 comprising a CD28 transmembrane domain (CD28 TM), CD28 intracellular domain (CD28 ICD), and a CD3 zeta intracellular signaling domain.

Embodiment 30. The CAR of embodiment 25 or 29 comprising CD28 TM of SEQ ID NO: 50, CD28 ICD of SEQ ID NO: 51, and a CD3 zeta intracellular signaling domain of SEQ ID NO: 53.

Embodiment 31. The CAR of any one of embodiments 25, 29, or 30, comprising the amino acid sequence of SEQ ID NO: 40 or 41.

Embodiment 32. The CAR of embodiment 28 or 31, encoded by a nucleic acid molecule comprising SEQ ID NO: 34, 35, 36, or 37.

Embodiment 33. A method of treating a subject with prostate cancer comprising administering an effective amount of the scFv of any one of embodiments 1-13, the scFab of any one of embodiments 14-21, the antibody of embodiment 22, or the CAR of any one of embodiments 25-32.

Embodiment 34. The method of embodiment 33, wherein cancer is localized (stages I and II), locally advanced (stage III), or advanced (stage IV) prostate cancer.

Embodiment 35. Use of the scFv of any one of embodiments 1-13, the scFab of any one of embodiments 14-21, the antibody of embodiment 22, or the CAR of any one of embodiments 25-32 in treating prostate cancer.

Embodiment 36. The use of embodiment 35, wherein cancer is localized (stages I and II), locally advanced (stage III), or advanced (stage IV) prostate cancer.

Embodiment 37. A lentiviral vector comprising a nucleic acid sequence encoding the scFv of any one of embodiments 1-13.

Embodiment 38. A lentiviral vector comprising a nucleic acid sequence encoding the CAR of any one of embodiments 25-32.

Embodiment 39. A lentivirus comprising the lentiviral vector of embodiment 37 or 38.

Embodiment 40. A eukaryotic cell comprising the lentiviral vector of embodiment 37 or 38.

Embodiment 41. A mammalian T cell comprising the lentiviral vector of embodiment 37 or 38.

Embodiment 42. A mammalian T cell comprising a chimeric antigen receptor (CAR) comprising the scFv of any one of embodiments 1-13.

Embodiment 43. A mammalian T cell comprising the CAR of any one of embodiments 25-32.

Embodiment 44. The mammalian T cell of embodiment 42 or 43, wherein the CAR comprises the amino acid sequence of SEQ ID NO: 38, 39, 40, or 41.

Embodiment 45. A method of treating a subject with prostate cancer comprising administering to the subject an effective amount of the mammalian T cell of any one of embodiments 41-44.

Embodiment 46. The method of embodiment 45, wherein the subject is a human.

Embodiment 47. The method of embodiment 45 or 46, wherein the mammalian T cell is a human T cell.

Embodiment 48. The method of any one of embodiments 45-47, wherein the effective amount of the mammalian T cell comprises between 1×10³ and 5×10⁻⁸ of the mammalian T cells.

Embodiment 49. The method of any one of embodiments 45-48, wherein the prostate cancer is localized (stages I and II), locally advanced (stage III), or advanced (stage IV) prostate cancer.

It is to be understood that while the disclosure has been described in conjunction with the preferred specific embodiments thereof, that the foregoing description and the examples are intended to illustrate and not limit the scope of the disclosure. It will be understood by those skilled in the art that various changes may be made, and equivalents may be substituted without departing from the scope of the disclosure, and further that other aspects, advantages and modifications will be apparent to those skilled in the art to which the disclosure pertains. In addition to the embodiments described herein, the present disclosure contemplates and claims those inventions resulting from the combination of features of the disclosure cited herein. Similarly, it will be appreciated that any described material, feature, or article may be used in combination with any other material, feature, or article, and such combinations are considered within the scope of this disclosure.

What is claimed:

1. A single chain variable fragment (scFv), comprising a heavy chain variable region, a linker region, and a light chain variable region, wherein:
the heavy chain variable region comprises heavy chain complementarity determining region 1 (CDR1), CDR2, and CDR3 of SEQ ID NOs: 8, 9, and 11, and the light chain variable region comprises light chain CDR1, CDR2, and CDR3 of SEQ ID NOs: 12, 14, and 15;
the heavy chain variable region comprises heavy chain CDR1, CDR2, and CDR3 of SEQ ID NOs: 7, 9, and 11, and the light chain variable region comprises light chain CDR1, CDR2, and CDR3 of SEQ ID NOs: 12, 14, and 15; or
the heavy chain variable region comprises heavy chain CDR1, CDR2, and CDR3 of SEQ ID NOs: 7, 10, and 11, and the light chain variable region comprises light chain CDR1, CDR2, and CDR3 of SEQ ID NOs: 13, 14, and 16.

2. The scFv of claim 1, wherein the heavy chain variable region comprises SEQ ID NO: 1 and the light chain variable region comprises SEQ ID NO: 2.

3. The scFv of claim 1, wherein the heavy chain variable region comprises SEQ ID NO: 3 and the light chain variable region comprises SEQ ID NO: 4.

4. The scFv of claim 1, wherein the heavy chain variable region comprises SEQ ID NO: 5 and the light chain variable region comprises SEQ ID NO: 6.

5. The scFv of claim 1, wherein the heavy chain variable region, the light chain variable region, or both the heavy chain variable region and the light chain variable region is humanized.

6. The scFv of claim 5, wherein the heavy chain variable region comprises SEQ ID NO: 17 and the light chain variable region comprises SEQ ID NO: 18.

7. The scFv of claim 5, wherein framework regions of the heavy chain variable region have about 95% sequence identity to the framework regions of SEQ ID NO: 23 or SEQ ID NO: 24.

8. The scFv of claim 1, wherein the linker region comprises SEQ ID NO: 25 or SEQ ID NO: 26.

9. The scFv of claim 1 fused to a transmembrane domain.

10. The scFv of claim 1, fused to a transmembrane domain and a signal transduction domain.

11. The scFv of claim 1, fused to transmembrane domain and an immune-costimulatory domain.

12. The scFv of claim 11, wherein the co-stimulatory domain comprises the amino acid sequence of SEQ ID NO: 42.

13. The scFv of claim 11, wherein the co-stimulatory domain comprises the amino acid sequence of SEQ ID NO: 43.

14. A single chain antigen binding fragment (scFab) comprising a heavy chain region, a linker region, and a light chain region, the light chain region comprising a light chain variable region and a light chain constant region, wherein
the heavy chain region comprises heavy chain CDR1, CDR2, and CDR3 of SEQ ID NOs: 8, 9, and 11, and the light chain variable region comprises light chain CDR1, CDR2, and CDR3 of SEQ ID NOs: 12, 14, and 15;
the heavy chain region comprises heavy chain CDR1, CDR2, and CDR3 of SEQ ID NOs: 7, 9, and 11, and the light chain variable region comprises light chain CDR1, CDR2, and CDR3 of SEQ ID NOs: 12, 14, and 15; or
the heavy chain region comprises heavy chain CDR1, CDR2, and CDR3 of SEQ ID NOs: 7, 10, and 11, and the light chain variable region comprises light chain CDR1, CDR2, and CDR3 of SEQ ID NOs: 13, 14, and 16.

15. The scFab of claim 14, wherein the heavy chain region comprises SEQ ID NO: 1 and the light chain region comprises SEQ ID NO: 2.

16. The scFab of claim 14, wherein the heavy chain region comprises SEQ ID NO: 3 and the light chain region comprises SEQ ID NO: 4.

17. The scFab of claim 14, wherein the heavy chain region comprises SEQ ID NO: 5 and the light chain region comprises SEQ ID NO: 6.

18. The scFab of claim 14, wherein the heavy chain region, the light chain region, or both the heavy chain region and the light chain region is humanized.

19. The scFab of claim 18, wherein the heavy chain region comprises SEQ ID NO: 17 and the light chain region comprises SEQ ID NO: 18.

20. The scFab of claim 18, wherein framework regions of the heavy chain region have about 95% sequence identity to the framework regions of SEQ ID NO: 23 or SEQ ID NO: 24.

21. The scFab of claim 14, wherein the linker region comprises SEQ ID NO: 25 or SEQ ID NO: 26.

22. An antibody comprising a heavy chain having an amino acid sequence according to SEQ ID NO: 19 and a light chain having an amino acid sequence according to SEQ ID NO: 21.

23. A composition comprising the scFv of claim 1.

24. The composition of claim 23, wherein the heavy chain variable region, the light chain variable region, or both the heavy chain variable region and the light chain variable region is humanized.

25. A chimeric antigen receptor (CAR) comprising the scFv of claim 1.

26. The CAR of claim 25 comprising a CD8 transmembrane domain (CD8 TM), 4-1BB intracellular domain (4-1BB ICD), and a CD3 zeta intracellular signaling domain.

27. The CAR of claim 25 comprising CD8 TM of SEQ ID NO: 48, 4-1BB ICD of SEQ ID NO: 49, and a CD3 zeta intracellular signaling domain of SEQ ID NO: 52.

28. The CAR of claim 25, comprising the amino acid sequence of SEQ ID NO: 38 or 39.

29. The CAR of claim 25 comprising a CD28 transmembrane domain (CD28 TM), CD28 intracellular domain (CD28 ICD), and a CD3 zeta intracellular signaling domain.

30. The CAR of claim 25 comprising CD28 TM of SEQ ID NO: 50, CD28 ICD of SEQ ID NO: 51, and a CD3 zeta intracellular signaling domain of SEQ ID NO: 53.

31. The CAR of claim 25, comprising the amino acid sequence of SEQ ID NO: 40 or 41.

32. The CAR of claim 28, encoded by a nucleic acid molecule comprising SEQ ID NO: 34, 35, 36, or 37.

33. A method of treating a subject with prostate cancer comprising administering an effective amount of the scFv of claim 1.

34. The method of claim 33, wherein cancer is localized (stages I and II), locally advanced (stage III), or advanced (stage IV) prostate cancer.

35. A lentiviral vector comprising a nucleic acid sequence encoding the scFv of claim 1.

36. A lentiviral vector comprising a nucleic acid sequence encoding the CAR of claim 25.

37. A lentivirus comprising the lentiviral vector of claim 35.

38. A eukaryotic cell comprising the lentiviral vector of claim 35.

39. A mammalian T cell comprising the lentiviral vector of claim 35.

40. A mammalian T cell comprising a chimeric antigen receptor (CAR) comprising the scFv of claim 1.

41. A mammalian T cell comprising the CAR of claim 25.

42. The mammalian T cell of claim 40, wherein the CAR comprises the amino acid sequence of SEQ ID NO: 38, 39, 40, or 41.

43. A method of treating a subject with prostate cancer comprising administering to the subject an effective amount of the mammalian T cell of claim 39.

44. The method of claim 43, wherein the subject is a human.

45. The method of claim 43, wherein the mammalian T cell is a human T cell.

46. The method of claim 43, wherein the effective amount of the mammalian T cell comprises between 1×10³ and 5×10⁻⁸ of the mammalian T cells.

47. The method of claim 43, wherein the prostate cancer is localized (stages I and II), locally advanced (stage III), or advanced (stage IV) prostate cancer.


SEQUENCE LISTING (53 sequences)

SEQ ID NO: 1 - protein, 116 aa, synthetic construct
QVQLKESGPGLVAPSQSLSITCTVSGFSLTYYGVHWGRQSPGKGLEWLGIIWAGGNTNYNSTLKSRLSISKDNSQSQVFLKMTSLQTDDTAMYYCARDDYAAMDYWGQGTSVTVSS

SEQ ID NO: 2 - protein, 112 aa, synthetic construct
DVVMTQTPLSLPVSLGDQASISCRSSQTLVHSNGNTFLHWYLKKPGQSPKLLIYKVSNRFSGVPDRFSGSGSGTHFTLKISRVEAEDLGVYFCSQGTHAPFTFGGGTKLEIK

SEQ ID NO: 3 - protein, 111 aa, synthetic construct
EVKLVESGPGLVAPSQSLSITCTVSGFSLTYYGVHWGRQSPGKGLEWLGIIWAGGNTNYNSTLKSRLSISKDNSQSQVFLKMTSLQTDDTAMYYCARDDYAAMDYWGQGTS

SEQ ID NO: 4 - protein, 114 aa, synthetic construct
ELVMTQSPLSLPVSLGDQASISCRSSQTLVHSNGNTFLHWYLKKPGQSPKLLIYKVSNRFSGVPDRFSGSGSGTHFTLKISRVEAEDLGVYFCSQGTHAPFTFGGGTKLEIKRA

SEQ ID NO: 5 - protein, 116 aa, synthetic construct
QVQLKESGPGLVAPSQSLSITCTVSGFSLTYYGVHWVRQPPGKGLEWLGIIWAGENTNYNSALMSRLSISKDNSKSQVFLKVNSLQTDDTAIYYCARDDYAAMDYWGQGTSVTVSS

SEQ ID NO: 6 - protein, 112 aa, synthetic construct
DVVMTQTPLSLPVSLGDQASISCRSSQSLVHSNGNTFLHWYLQRPGQSPKLLIYKVSNRFSGVPDRFSGSGSGTDFTLKISRVEAEDLGVYFCSQGSHVPFTFGGGTKLEIK

SEQ ID NO: 7 - protein, 8 aa, synthetic construct
GFSLTYYG

SEQ ID NO: 8 - protein, 8 aa, synthetic construct
GFTFTYYG

SEQ ID NO: 9 - protein, 7 aa, synthetic construct
IWAGGNT

SEQ ID NO: 10 - protein, 7 aa, synthetic construct
IWAGENT

SEQ ID NO: 11 - protein, 10 aa, synthetic construct
ARDDYAAMDY

SEQ ID NO: 12 - protein, 11 aa, synthetic construct
QTLVHSNGNTF

SEQ ID NO: 13 - protein, 11 aa, synthetic construct
QSLVHSNGNTF

SEQ ID NO: 14 - skipped

SEQ ID NO: 15 - protein, 9 aa, synthetic construct
SQGTHAPFT

SEQ ID NO: 16 - protein, 9 aa, synthetic construct
SQGSHVPFT

SEQ ID NO: 17 - protein, 116 aa, synthetic construct
QVQLQESGPGLVRPSQTLSLTCTVSGFTFTYYGVHWVRQPPGRGLEWIGIIWAGGNTNYNSTLKSRVTMLVDTSKSQFSLRLSSVTAADTAVYYCARDDYAAMDYWGQGSLVTVSS

SEQ ID NO: 18 - protein, 112 aa, synthetic construct
DVVMTQSPSSLSVTLGQPASISCRSSQTLVHSNGNTFLHWFQQRPGQSPRRLIYKVSNRFSGVPDRFSGSGSGTDFTLTISRVEAEDVGVYYCSQGTHAPFTFGGGTKVEIK

SEQ ID NO: 19 - protein, 447 aa, synthetic construct
QVQLQESGPGLVRPSQTLSLTCTVSGFTFTYYGVHWVRQPPGRGLEWIGIIWAGGNTNYNSTLKSRVTMLVDTSKSQFSLRLSSVTAADTAVYYCARDDYAAMDYWGQGSLVTVSSAAATKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 20 - DNA, 1429 bp, synthetic construct
  source 1..1429: mol_type = other DNA
caggtccaactgcaggaatccggtccgggtctggtgcgtccgtcgcaaaccctgagcctgacctgtacggttagcggctttaccttcacgtattacggtgtgcactgggttcgtcagccgccgggtcgtggtctggaatggatcggtattatctgggcgggtggcaacacgaattacaactctaccctgaaaagtcgtgtcacgatgctggttgatacctcgaaaagccaattcagcctgcgtctgtcatcggtgaccgcagcagatacggcagtttattactgtgctcgtgatgactatgcagctatggactactggggccagggtagcctggttaccgtcagctctgcggccgcaaccaagggcccatcggtcttccccctggcaccctcctccaagagcacctctgggggcacagcagccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacacagaagagcctctccctgtctccgggtaaatgattctagagggcccgaacaaaaactcatctcagaagaggatctgaatagcgccgtcgaccatcatcatcatcatcatgagttagca

SEQ ID NO: 21 - protein, 219 aa, synthetic construct
DVVMTQSPSSLSVTLGQPASISCRSSQTLVHSNGNTFLHWFQQRPGQSPRRLIYKVSNRFSGVPDRFSGSGSGTDFTLTISRVEAEDVGVYYCSQGTHAPFTFGGGTKVEIKRRVATPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 22 - DNA, 657 bp, synthetic construct
  source 1..657: mol_type = other DNA
gatgtcgtgatgacgcagtcgccttctagtctgagcgtgaccctgggacagcccgcctcgatttcgtgtaggagttcccagacgctggtgcactctaacggcaataccttcctgcattggtttcagcagaggcctggacagagcccacgccggctgatctacaaggtgtccaaccgattcagtggcgtccctgaccgctttagtggatctggctcgggaacagatttcaccctgacaattagtagagtggaggccgaagacgtgggcgtctactattgcagccagggaacccacgctcccttcacatttggcggaggcactaaggtcgagatcaaaaggagagtggccaccccaagcgtcttcatttttcccccatccgacgaacagctgaagtctggcaccgcctcggtggtctgtctgctgaacaacttctacccaagggaggccaaggtgcagtggaaagtcgataacgctctgcagagcggaaattcccaggagagtgtgacagaacaggactctaaggattcgacttatagcctgagctccactctgacgctgagcaaagccgattacgagaagcataaagtgtatgcttgcgaagtgacgcaccagggactgagttcgcccgtgacaaaatcgttcaacagaggagaatgc

SEQ ID NO: 23 - protein, 111 aa, synthetic construct
  VARIANT 28..36: note = any amino acid
  VARIANT 52..64: note = any amino acid
  VARIANT 98..103
EVKLVESGGGLVQPGGSLRLSCAASGFXXXXXXXXWVRQAPGKGLEWVGAIXXXXXXXXXXXXKGRFTISRDNSKNTLYLQMNSLRAEDTAVYYCARXXXXXFDYWGQGTL

SEQ ID NO: 24 - protein, 111 aa, synthetic construct
  VARIANT 28..36: note = any amino acid
  VARIANT 52..64: note = any amino acid
EVQLVESGGGLVQPGGSLRLSCAASGFXXXXXXXXWVRQAPGKGLEWVGAIXXXXXXXXXXXXKGRFTISRDNSKNTLYLQMNSLRAEDTAVYYCARXXXXXFDYWGQGTL

SEQ ID NO: 25 - protein, 20 aa, synthetic construct
GGGGSGGGGSGGGGSGGGGS

SEQ ID NO: 26 - protein, 30 aa, synthetic construct
GGGGSGGGGSGGGGSGGGGSGGGGSGGGGS

SEQ ID NO: 27 - protein, 248 aa, synthetic construct
EVKLVESGPGLVAPSQSLSITCTVSGFSLTYYGVHWGRQSPGKGLEWLGIIWAGGNTNYNSTLKSRLSISKDNSQSQVFLKMTSLQTDDTAMYYCARDDYAAMDYWGQGTSGGGGSGGGGSGGGGSGGGGSSMADVVMTQSPSSLSVTLGQPASISCRSSQTLVHSNGNTFLHWFQQRPGQSPRRLIYKVSNRFSGVPDRFSGSGSGTDFTLTISRVEAEDVGVYYCSQGTHAPFTFGGGTKVEIKRR

SEQ ID NO: 28 - protein, 471 aa, synthetic construct
EVKLVESGPGLVAPSQSLSITCTVSGFSLTYYGVHWGRQSPGKGLEWLGIIWAGGNTNYNSTLKSRLSISKDNSQSQVFLKMTSLQTDDTAMYYCARDDYAAMDYWGQGSLVTVSSAAATKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSGGGGSGGGGSGGGGSGGGGSGGGGSGGGGSSMADVVMTQSPSSLSVTLGQPASISCRSSQTLVHSNGNTFLHWFQQRPGQSPRRLIYKVSNRFSGVPDRFSGSGSGTDFTLTISRVEAEDVGVYYCSQGTHAPFTFGGGTKVEIKRRVATPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 29 - protein, 110 aa, synthetic construct
EVKLVESGPGLVAPSQSLSITCTVSGFSLTYYGVHWGRQSPGKGLEWLGIIWAGGNTNYNSTLKSRLSISKDNSQSQVFLKMTSLQTDDTAMYYCARDDYAAMDYWGQGT

SEQ ID NO: 30 - protein, 112 aa, synthetic construct
ELVMTQSPLSLPVSLGDQASISCRSSQTLVHSNGNTFLHWYLKKPGQSPKLLIYKVSNRFSGVPDRFSGSGSGTHFTLKISRVEAEDLGVYFCSQGTHAPFTFGGGTKLEIK

SEQ ID NO: 31 - protein, 112 aa, synthetic construct
QVQLQESGPGLVKPSETLSLTCTVSGGSISSYYWSWIRQPPGKGLEWIGYIYTSGSTNYNPSLKSRVTISVDTSKNQFSLKLSSVTAADTAVYYCARYFDYWGQGSLVTVSS

SEQ ID NO: 32 - protein, 112 aa, synthetic construct
DVVMTQSPLSLPVTLGQPASISCRSSQSLVHSDGNTYLNWFQQRPGQSPRRLIYKVSNRDSGVPDRFSGSGSGTDFTLKISRVEAEDVGVYYCMQGTHWPCTFGQGTKLEIK

SEQ ID NO: 33 - protein, 111 aa, synthetic construct
EVQLVESGPGLVAPSQSLSITCTVSGFSLTYYGVHWGRQSPGKGLEWLGIIWAGGNTNYNSTLKSRLSISKDNSKSQVFLKMNSLQTDDTAMYYCARDDYAAMDYWGQGTS

SEQ ID NO: 34 - DNA, 1410 bp, synthetic construct
  source 1..1410: mol_type = other DNA
caagtgcagctgaaagaaagcggacccggtctggtggcccccagccagtctttaagcattacttgtaccgtgagcggcttttctttaacctactacggcgtccactgggtgagacagcctcccggcaagggactggagtggctgggcatcatctgggccggcgagaacaccaactacaacagcgctttaatgtctcgtctgagcatcagcaaggacaacagcaagagccaagtttttttaaaggtgaactctttacagaccgacgacaccgctatctactactgcgctcgtgacgactacgccgccatggactactggggccaaggtacaagcgtgacagtgtccggcggaggcggaagcggcggcggcggcagcagcggcggaggcagcgacgtggtgatgacccagacacctttaagcctccccgtttctttaggcgatcaagcttccatcagctgtcgtagcagccaatctttagtgcacagcaatggcaacacctttttacactggtatttacagagacccggacagagccccaagctgctgatctacaaggtgtccaatcgtttcagcggcgtccccgatcgttttagcggcagcggcagcggaaccgatttcactttaaagatttctcgtgtggaggccgaggacctcggcgtgtacttctgtagccaaggtagccacgtgcccttcacattcggcggcggcaccaagctggagatcaagagagccgctagcaccactaccccagcaccgaggccacccaccccggctcctaccatcgcctcccagcctctgtccctgcgtccggaggcatgtagacccgcagctggtggggccgtgcatacccggggtcttgacttcgcctgcgatatctacatttgggcccctctggctggtacttgcggggtcctgctgctttcactcgtgatcactctttactgtaagcgcggtcggaagaagctgctgtacatctttaagcaacccttcatgaggcctgtgcagactactcaagaggaggacggctgttcatgccggttcccagaggaggaggaaggcggctgcgaactgcgcgtgaaattcagccgcagcgcagatgctccagcctacaagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcggtga

SEQ ID NO: 35 - DNA, 1410 bp, synthetic construct
  source 1..1410: mol_type = other DNA
gacgtggtgatgacccagacccctctgagcctgcctgtgagcctgggcgaccaggccagcatcagctgcaggagcagccagagcctggtgcacagcaacggcaacaccttcctgcactggtacctgcagaggcctggccagagccctaagctgctgatctacaaggtgagcaacaggttcagcggcgtgcctgacaggttcagcggcagcggcagcggcaccgacttcaccctgaagatcagcagggtggaggccgaggacctgggcgtgtacttctgcagccagggcagccacgtgcctttcaccttcggcggcggcaccaagctggagatcaagagggccggcggcggcggcagcggcggcggcggcagcagcggcggcggcagccaggtgcagctgaaggagagcggccctggcctggtggcccctagccagagcctgagcatcacctgcaccgtgagcggcttcagcctgacctactacggcgtgcactgggtgaggcagcctcctggcaagggcctggagtggctgggcatcatctgggccggcgagaacaccaactacaacagcgccctgatgagcaggctgagcatcagcaaggacaacagcaagagccaggtgttcctgaaggtgaacagcctgcagaccgacgacaccgccatctactactgcgccagggacgactacgccgccatggactactggggccagggcaccagcgtgaccgtgagcgctagcaccactaccccagcaccgaggccacccaccccggctcctaccatcgcctcccagcctctgtccctgcgtccggaggcatgtagacccgcagctggtggggccgtgcatacccggggtcttgacttcgcctgcgatatctacatttgggcccctctggctggtacttgcggggtcctgctgctttcactcgtgatcactctttactgtaagcgcggtcggaagaagctgctgtacatctttaagcaacccttcatgaggcctgtgcagactactcaagaggaggacggctgttcatgccggttcccagaggaggaggaaggcggctgcgaactgcgcgtgaaattcagccgcagcgcagatgctccagcctacaagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcggtga

SEQ ID NO: 36 - DNA, 1416 bp, synthetic construct
  source 1..1416: mol_type = other DNA
caagtgcagctgaaagaaagcggacccggtctggtggcccccagccagtctttaagcattacttgtaccgtgagcggcttttctttaacctactacggcgtccactgggtgagacagcctcccggcaagggactggagtggctgggcatcatctgggccggcgagaacaccaactacaacagcgctttaatgtctcgtctgagcatcagcaaggacaacagcaagagccaagtttttttaaaggtgaactctttacagaccgacgacaccgctatctactactgcgctcgtgacgactacgccgccatggactactggggccaaggtacaagcgtgacagtgtccggcggaggcggaagcggcggcggcggcagcagcggcggaggcagcgacgtggtgatgacccagacacctttaagcctccccgtttctttaggcgatcaagcttccatcagctgtcgtagcagccaatctttagtgcacagcaatggcaacacctttttacactggtatttacagagacccggacagagccccaagctgctgatctacaaggtgtccaatcgtttcagcggcgtccccgatcgttttagcggcagcggcagcggaaccgatttcactttaaagatttctcgtgtggaggccgaggacctcggcgtgtacttctgtagccaaggtagccacgtgcccttcacattcggcggcggcaccaagctggagatcaagagagccgctagcaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccctgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 37 - DNA, 1416 bp, synthetic construct
  source 1..1416: mol_type = other DNA
gacgtggtgatgacccagacccctctgagcctgcctgtgagcctgggcgaccaggccagcatcagctgcaggagcagccagagcctggtgcacagcaacggcaacaccttcctgcactggtacctgcagaggcctggccagagccctaagctgctgatctacaaggtgagcaacaggttcagcggcgtgcctgacaggttcagcggcagcggcagcggcaccgacttcaccctgaagatcagcagggtggaggccgaggacctgggcgtgtacttctgcagccagggcagccacgtgcctttcaccttcggcggcggcaccaagctggagatcaagagggccggcggcggcggcagcggcggcggcggcagcagcggcggcggcagccaggtgcagctgaaggagagcggccctggcctggtggcccctagccagagcctgagcatcacctgcaccgtgagcggcttcagcctgacctactacggcgtgcactgggtgaggcagcctcctggcaagggcctggagtggctgggcatcatctgggccggcgagaacaccaactacaacagcgccctgatgagcaggctgagcatcagcaaggacaacagcaagagccaggtgttcctgaaggtgaacagcctgcagaccgacgacaccgccatctactactgcgccagggacgactacgccgccatggactactggggccagggcaccagcgtgaccgtgagcgctagcaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccctgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 38 - protein, 467 aa, synthetic construct
QVQLKESGPGLVAPSQSLSITCTVSGFSLTYYGVHWVRQPPGKGLEWLGIIWAGENTNYNSALMSRLSISKDNSKSQVFLKVNSLQTDDTAIYYCARDDYAAMDYWGQGTSVTVSGGGGSGGGGSSGGGSDVVMTQTPLSLPVSLGDQASISCRSSQSLVHSNGNTFLHWYLQRPGQSPKLLIYKVSNRFSGVPDRFSGSGSGTDFTLKISRVEAEDLGVYFCSQGSHVPFTFGGGTKLEIKRATTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 39 - protein, 469 aa, synthetic construct
DVVMTQTPLSLPVSLGDQASISCRSSQSLVHSNGNTFLHWYLQRPGQSPKLLIYKVSNRFSGVPDRFSGSGSGTDFTLKISRVEAEDLGVYFCSQGSHVPFTFGGGTKLEIKRAGGGGSGGGGSSGGGSQVQLKESGPGLVAPSQSLSITCTVSGFSLTYYGVHWVRQPPGKGLEWLGIIWAGENTNYNSALMSRLSISKDNSKSQVFLKVNSLQTDDTAIYYCARDDYAAMDYWGQGTSVTVSASTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 40 - protein, 469 aa, synthetic construct
QVQLKESGPGLVAPSQSLSITCTVSGFSLTYYGVHWVRQPPGKGLEWLGIIWAGENTNYNSALMSRLSISKDNSKSQVFLKVNSLQTDDTAIYYCARDDYAAMDYWGQGTSVTVSGGGGSGGGGSSGGGSDVVMTQTPLSLPVSLGDQASISCRSSQSLVHSNGNTFLHWYLQRPGQSPKLLIYKVSNRFSGVPDRFSGSGSGTDFTLKISRVEAEDLGVYFCSQGSHVPFTFGGGTKLEIKRATTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 41 - protein, 469 aa, synthetic construct
DVVMTQTPLSLPVSLGDQASISCRSSQSLVHSNGNTFLHWYLQRPGQSPKLLIYKVSNRFSGVPDRFSGSGSGTDFTLKISRVEAEDLGVYFCSQGSHVPFTFGGGTKLEIKRAGGGGSGGGGSSGGGSQVQLKESGPGLVAPSQSLSITCTVSGFSLTYYGVHWVRQPPGKGLEWLGIIWAGENTNYNSALMSRLSISKDNSKSQVFLKVNSLQTDDTAIYYCARDDYAAMDYWGQGTSVTVSTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 42 - protein, 178 aa, synthetic construct
IYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 43 - protein, 180 aa, synthetic construct
FWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 44 - protein, 15 aa, synthetic construct
GGGGSGGGGSSGGGS

SEQ ID NO: 45 - protein, 115 aa, synthetic construct
QVQLKESGPGLVAPSQSLSITCTVSGFSLTYYGVHWVRQPPGKGLEWLGIIWAGENTNYNSALMSRLSISKDNSKSQVFLKVNSLQTDDTAIYYCARDDYAAMDYWGQGTSVTVS

SEQ ID NO: 46 - protein, 114 aa, synthetic construct
DVVMTQTPLSLPVSLGDQASISCRSSQSLVHSNGNTFLHWYLQRPGQSPKLLIYKVSNRFSGVPDRFSGSGSGTDFTLKISRVEAEDLGVYFCSQGSHVPFTFGGGTKLEIKRA

SEQ ID NO: 47 - protein, 45 aa, synthetic construct
TTTPAPRPPTPAPTIASQPLSLRPEACRPAAGGAVHTRGLDFACD

SEQ ID NO: 48 - protein, 24 aa, synthetic construct
IYIWAPLAGTCGVLLLSLVITLYC

SEQ ID NO: 49 - protein, 42 aa, synthetic construct
KRGRKKLLYIFKQPFMRPVQTTQEEDGCSCRFPEEEEGGCEL

SEQ ID NO: 50 - protein, 27 aa, synthetic construct
FWVLVVVGGVLACYSLLVTVAFIIFWV

SEQ ID NO: 51 - protein, 41 aa, synthetic construct
RSKRSRLLHSDYMNMTPRRPGPTRKHYQPYAPPRDFAAYRS

SEQ ID NO: 52 - protein, 112 aa, synthetic construct
RVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 53 - protein, 112 aa, synthetic construct
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR